(19) United States
(12) Patent Application Publication
(10) Pub. No.: US 2025/0177115 A1
(43) Pub. Date: Jun. 5, 2025

(54) COMBINATORIAL THERAPIES INCLUDING IMPLANTABLE DAMPING DEVICES AND THERAPEUTIC AGENTS FOR TREATING A CONDITION AND ASSOCIATED SYSTEMS AND METHODS OF USE

(71) Applicant: THE BRAIN PROTECTION COMPANY PTY LTD, New South Wales (AU)

(72) Inventors: David Stephen Celermajer, Vaucluse (AU); Zoran Milijasevic, Bayview (AU); Anthony John Ujhazy, East Lindfield (AU); Mark Carnegie, Takaka (NZ); Rachel Ashley Levin, North Sydney (AU)

(21) Appl. No.: 18/683,344

(22) PCT Filed: Aug. 19, 2022
(86) PCT No.: PCT/AU2022/050928

§ 371 Date: Feb. 13, 2024

Related U.S. Application Data
(60) Provisional application No. 63/235,506, filed on Aug. 20, 2021.

(51) Int. Cl. A61F 2/07; A61F 2/06; A61L 31/16

(52) U.S. Cl. CPC A61F 2/07; A61L 31/16; A61F 2002/068; A61F 2230/001; A61F 2230/0069; A61F 2250/0039; A61L 2300/216; A61L 2300/222; A61L 2300/406; A61L 2300/416; A61L 2400/16

(57) ABSTRACT

Devices, systems, and methods for combinatorial treatment of a condition with an implantable damping device and therapeutic agent are disclosed herein. Methods for treating one or more effects of the condition, such as a neurological condition, include providing the implantable damping device and at least one other therapy, such as a therapeutic agent, that treats the condition to the patient. The implantable damping device includes a flexible damping member and an abating substance and can be placed in apposition with a blood vessel. The flexible damping member forms a generally tubular structure having an inner and an outer surface, the inner surface formed of a sidewall having a partially deformable portion. The abating substance is disposed within the partially deformable portion and moves longitudinally and/or radially within the partially deformable portion in response to pulsatile blood flow.

### CROSS-REFERENCE TO RELATED APPLICATION(S)

This application claims priority to U.S. Patent Application No. 63/235,506, entitled “COMBINATORIAL THERAPIES INCLUDING IMPLANTABLE DAMPING DEVICES AND THERAPEUTIC AGENTS FOR TREATING A CONDITION AND ASSOCIATED SYSTEMS AND METHODS OF USE,” filed on Aug. 20, 2021, which is herein incorporated by reference in its entirety.

### TECHNICAL FIELD

The present technology relates to combinatorial therapies including an implantable damping device and therapeutic agents for treating a condition (e.g., a neurodegenerative condition such as dementia) and associated systems and methods of use. In particular, the present technology is directed to combinatorial therapies including an implantable damping device for positioning at, near, within, around, or in place of at least a portion of an artery and one or more therapeutic agents (e.g., EDG Receptor Family modulators, MMP inhibitors, and senolytic agents) for treating the condition.

### BACKGROUND

Any discussion of the prior art throughout the specification should in no way be considered as an admission that such prior art is widely known or forms part of common general knowledge in the field.

The heart supplies oxygenated blood to the body through a network of interconnected, branching arteries starting with the largest artery in the body—the aorta. As shown in the schematic view of the heart and selected arteries in FIG. 1A, the portion of the aorta closest to the heart is divided into three regions: the ascending aorta (where the aorta initially leaves the heart and extends in a superior direction), the aortic arch, and the descending aorta (where the aorta extends in an inferior direction). Three major arteries branch from the aorta along the aortic arch: the brachiocephalic artery, the left common carotid artery, and the left subclavian artery. The brachiocephalic artery extends away from the aortic arch and subsequently divides into the right common carotid artery, which supplies oxygenated blood to the head and neck, and the right subclavian artery, which predominantly supplies blood to the right arm. The left common carotid artery extends away from the aortic arch and supplies the head and neck. The left subclavian artery extends away from the aortic arch and predominantly supplies blood to the left arm. Each of the right common carotid artery and the left common carotid artery subsequently branches into separate internal and external carotid arteries.

During the systole stage of a heartbeat, contraction of the left ventricle forces blood into the ascending aorta that increases the pressure within the arteries (known as systolic blood pressure). The volume of blood ejected from the left ventricle creates a pressure wave—known as a pulse wave—that propagates through the arteries propelling the blood. The pulse wave causes the arteries to dilate, as shown schematically in FIG. 1B. When the left ventricle relaxes (the diastole stage of a heartbeat), the pressure within the arterial system decreases (known as diastolic blood pressure), which allows the arteries to contract.

The difference between the systolic blood pressure and the diastolic blood pressure is the “pulse pressure,” which generally is determined by the magnitude of the contraction force generated by the heart, the heart rate, the peripheral vascular resistance, and diastolic “run-off” (e.g., the blood flowing down the pressure gradient from the arteries to the veins), amongst other factors. High flow organs, such as the brain, are particularly sensitive to excessive pressure and flow pulsatility. To ensure a relatively consistent flow rate to such sensitive organs, the walls of the arterial vessels expand and contract in response to the pressure wave to absorb some of the pulse wave energy. As the vasculature ages, however, the arterial walls lose elasticity, which causes an increase in pulse wave speed and wave reflection through the arterial vasculature. Arterial stiffening impairs the ability of the carotid arteries and other large arteries to expand and dampen flow pulsatility, which results in an increase in systolic pressure and pulse pressure. Accordingly, as the arterial walls stiffen over time, the arteries transmit excessive force into the distal branches of the arterial vasculature.

Research suggests that consistently high systolic pressure, pulse pressure, and/or change in pressure over time (dP/dt) increases the risk of dementia, such as vascular dementia (e.g., an impaired supply of blood to the brain or bleeding within the brain). Without being bound by theory, it is believed that high pulse pressure can be the root cause or an exacerbating factor of vascular dementia and age-related dementia (e.g., Alzheimer's disease). As such, the progression of vascular dementia and age-related dementia (e.g., Alzheimer's disease) may also be affected by the loss of elasticity in the arterial walls and the resulting stress on the cerebral vessels. Alzheimer's Disease, for example, is generally associated with the presence of neuritic plaques and tangles in the brain. Recent studies suggest that increased pulse pressure, increased systolic pressure, and/or an increase in the rate of change of pressure (dP/dt) may, over time, cause microbleeds within the brain that may contribute to the neuritic plaques and tangles.

By 2050, it is estimated that at least one in every 85 people will be living with Alzheimer's disease world-wide and more than eight times as many people have shown preclinical symptoms. Additional disease-modifying therapies that will prevent or delay the onset or slow progression of neurological conditions, such as dementia, have been and are being developed. As of March 2020, there are 2,272 clinical trials and/or other related testing ongoing for treatment of Alzheimer's disease, one of several neurological conditions that is becoming increasingly more common as the world's population ages. While the therapeutic agents undergoing testing in these clinical trials may improve memory, behavior, cognition and/or reduce neuropsychiatric symptoms of Alzheimer's disease, additional studies testing the efficacy, safety, and tolerability of these therapeutic agents, and/or use of additional therapeutic agents are needed. Accordingly, there is a need for improved devices, systems, and methods for treating vascular and/or age-related dementia.

### SUMMARY OF THE INVENTION

According to a first aspect of the invention, there is provided a method for treating and/or preventing one or more effects of a condition in a subject in need thereof, the method comprising:
providing a device for treating and/or preventing one or more effects of the condition, and configured to be placed in apposition with a blood vessel, the device comprising:
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel; and
providing a therapy that treats or slows one or more effects of the condition in combination with the device, wherein the therapy is an EDG Receptor Family Modulator, an MMP inhibitor, or a senolytic agent or combination thereof.

According to a second aspect of the invention, there is provided a method for treating and/or preventing one or more effects of a condition in a subject in need thereof, the method comprising:
providing a device for treating and/or preventing one or more effects of the condition, and configured to be placed in apposition with a blood vessel, the device comprising:
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel; and
providing a therapy that treats or slows one or more effects of the condition in combination with the device, wherein the therapy is an EDG Receptor Family Modulator, an MMP inhibitor, or a senolytic agent or combination thereof.

According to a third aspect of the invention, there is provided a method for treating and/or preventing one or more effects of a condition in a subject in need thereof, the method comprising:
providing a therapy for treating and/or preventing one or more effects of the condition to the subject in need thereof, wherein the subject has previously been provided a device that treats or slows one or more effects of the condition and was placed in apposition with a blood vessel, the device comprising:
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel;
wherein the therapy is an EDG Receptor Family Modulator, an MMP inhibitor, or a senolytic agent or combination thereof.

According to a fourth aspect of the invention, there is provided a system for treating and/or preventing one or more effects of a condition in a subject in need thereof, the system comprising:
an effective amount of a therapy for treating and/or preventing one or more effects of the condition;
a device for treating and/or preventing one or more effects of the condition, the device comprising:
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel;
wherein the therapy is an EDG Receptor Family Modulator, an MMP inhibitor, or a senolytic agent or combination thereof.

According to a fifth aspect of the invention, there is provided a system for treating and/or preventing one or more effects of a condition in a subject in need thereof, the system comprising:
an effective amount of a therapy for treating and/or preventing one or more effects of the condition, wherein the therapy is an EDG Receptor Family Modulator, an MMP inhibitor, or a senolytic agent or combination thereof;
a device for treating and/or preventing one or more effects of the condition, the device comprising:
   a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
   an abating substance disposed within the one or more at least partially deformable portions of the sidewall configured to move longitudinally and/or radially within one partially deformable portion in response to pulsatile blood flow within the blood vessel;
wherein, the effective amount of the therapy for treating and/or preventing one or more effects of the condition is carried by or more of the at least partially deformable portions of the device, and
wherein, when the one or more at least partially deformable portions are at least partially deformed, the effective amount of therapy for treating and/or preventing one or more effects of the condition is released from the device.

According to a sixth aspect of the invention, there is provided a method for treating a patient having a condition comprising:
(a) determining or having determined whether the patient has an elevated pulse pressure or elevated pulse wave intensity/FCWI and a history of a microbleed, blood brain barrier dysfunction or permeability, systemic inflammation, blood brain barrier inflammation, neuroinflammation, increased level of at least one circulating or CSF cytokine, increased reactive oxygen species, or a combination thereof by:
   (i) obtaining or having obtained information which indicates that the patient has the elevated pulse pressure or elevated pulse wave intensity/FCWI and has previously had symptoms of a microbleed, blood brain barrier dysfunction or permeability, systemic inflammation, blood brain barrier inflammation, neuroinflammation, increased level of at least one circulating cytokine, increased reactive oxygen species, or a combination thereof and/or was previously diagnosed with a microbleed, blood brain barrier dysfunction or permeability, systemic inflammation, blood brain barrier inflammation, neuroinflammation, increased level of at least one circulating cytokine, increased reactive oxygen species or a combination thereof; and/or
   (ii) monitoring or having monitored the subject for the elevated pulse pressure and symptoms of a microbleed, blood brain barrier dysfunction or permeability, systemic inflammation, blood brain barrier inflammation, neuroinflammation, increased level of at least one circulating cytokine, increased reactive oxygen species or a combination thereof; and
(b) if the patient has previously had symptoms of a microbleed, blood brain barrier dysfunction or permeability, systemic inflammation, blood brain barrier inflammation, neuroinflammation, increased level of at least one circulating or CSF cytokine, increased reactive oxygen species, or a combination thereof, was previously diagnosed with a microbleed, blood brain barrier dysfunction or permeability, systemic inflammation, blood brain barrier inflammation, neuroinflammation, increased level of at least one circulating cytokine, increased reactive oxygen species, or a combination thereof, and/or symptoms of a microbleed, blood brain barrier dysfunction or permeability, systemic inflammation, blood brain barrier inflammation, neuroinflammation, increased level of at least one circulating cytokine, increased reactive oxygen species, or a combination thereof were monitored, then
   (i) providing an EDG Receptor Family Modulator, an MMP inhibitor, or a senolytic agent or combination thereof. to the patient, and
   (ii) providing a device for treating and/or preventing one or more effects of the condition, the device comprising:
      a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
      an abating substance disposed within the one or more at least partially deformable portions of the sidewall configured to move longitudinally and/or radially within one partially deformable portion in response to pulsatile blood flow within the blood vessel;
(c) if the patient has not had symptoms of a microbleed, APOE4 expression, blood brain barrier dysfunction or permeability, systemic inflammation, blood brain barrier inflammation, neuroinflammation, increased level of at least one circulating or CSF cytokine, increased reactive oxygen species, or a combination thereof, was not previously diagnosed with a microbleed, APOE4 expression, blood brain barrier dysfunction or permeability, systemic inflammation, blood brain barrier inflammation, neuroinflammation, increased level of at least one circulating or CSF cytokine, increased reactive oxygen species, or a combination thereof, then providing the device for treating and/or preventing one or more effects of the condition.

It is an object of the present invention to overcome or ameliorate at least one of the disadvantages of the prior art, or to provide a useful alternative.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a schematic illustration of a human heart and a portion of the arterial system near the heart.

FIG. 1B is a schematic illustration of a pulse wave propagating along a blood vessel.

FIG. 2A is a front view of a damping device in accordance with the present technology, shown in a deployed, relaxed state.

FIG. 2B is a front cross-sectional view of the damping device shown in FIG. 2A.

FIG. 2C is a front cross-sectional view of the damping device shown in FIG. 2A, shown in a deployed state positioned within a blood vessel.

FIG. 2D is a front cross-sectional view of another embodiment of a damping device in accordance with the present technology, shown in a deployed, relaxed state.

FIGS. 2E-2G are front cross-sectional views of several embodiments of damping members in accordance with the present technology, all shown in a deployed, relaxed state.

FIG. 3A is a front cross-sectional view of another embodiment of a damping device in accordance with the present technology shown in a deployed, relaxed state.

FIGS. 3B-3D are front cross-sectional views of several embodiments of damping members in accordance with the present technology, all shown in a deployed, relaxed state.

FIG. 4A is a front view of a damping device in accordance with another embodiment of the present technology, shown in a deployed, relaxed state.

FIG. 4B is a front cross-sectional view of the damping device shown in FIG. 4A.

FIG. 4C is a front cross-sectional view of the damping device shown in FIG. 4A, shown in a deployed state positioned within a blood vessel.

FIG. 4D is a front cross-sectional view of a portion of a damping member in accordance with the present technology showing deformation of the damping member (in dashed lines) in response to a pulse wave.

FIG. 4E is a front cross-sectional view of a portion of another damping member in accordance with the present technology showing deformation of the damping member (in dashed lines) in response to a pulse wave.

FIGS. 5-7 are front cross-sectional views of several embodiments of damping devices in accordance with the present technology.

FIGS. 8A-8E illustrate a method of delivering a damping device to an artery in accordance with the present technology.

FIGS. 9A-9F are schematic cross-sectional views of several embodiments of damping members in accordance with the present technology.

FIGS. 10 and 11 are front cross-sectional views of embodiments of damping devices shown positioned at or near a resected blood vessel in accordance with the present technology.

FIG. 12A is a front view of a helical damping device in accordance with the present technology, shown positioned around a blood vessel in a deployed, relaxed state.

FIG. 12B is a cross-sectional view of the damping device of FIG. 12A (taken along line 12B-12B in FIG. 12A), shown positioned around the blood vessel as a pulse pressure wave travels through the vessel.

FIGS. 13 and 14 show different embodiments of a wrapped damping device, each shown positioned around a blood vessel in accordance with the present technology.

FIG. 15 is a cross-sectional view of another embodiment of a damping device in accordance with the present technology.

FIG. 16A is a perspective view of another embodiment of a damping device in accordance with the present technology.

FIG. 16B is a cross-sectional view of the damping device shown in FIG. 16A, taken along line 16B-16B.

FIG. 17A is a perspective view of another embodiment of a damping device in accordance with the present technology.

FIG. 17B is a cross-sectional view of the damping device shown in FIG. 17A.

FIG. 18A is a perspective view of another embodiment of a damping device in accordance with the present technology.

FIG. 18B is a front view of the damping device shown in FIG. 18A, shown in a deployed state positioned around a blood vessel.

FIG. 19A is a perspective view of a damping device in accordance with another embodiment of the present technology, shown in an unwrapped state.

FIG. 19B is a top view of the damping device shown in FIG. 19A, shown in an unwrapped state.

FIG. 20 is a flow chart illustrating a method in accordance with the present technology.

FIG. 21 is a graph showing body weight at weekly intervals, from one week prior to TAC surgery (W-1), to six weeks after TAC surgery (W6) according to some embodiments. Data are mean±SEM (*: p<0.05 vs. plain suture, by unpaired t-test).

FIG. 22A is a graph showing echocardiogram data assessing peak velocity in mice receiving a plain suture according to some embodiments.

FIG. 22B is a graph showing echocardiogram data assessing peak velocity in mice receiving an absorbable suture according to some embodiments.

FIG. 23 is a dot plot showing the % alterations in a Y maze in mice with an absorbable suture versus a plain suture, without treatment according to some embodiments. Measurement of the number of alternations within the 3 arms of a Y-Maze according to the following formula: % alternation=(total alternations/(total entries−2)×100). The absorbable suture group had a mean % alteration of 61.63 (Std. Dev=13.25, Std. Error of Mean (SEM)=3.675, n=13), and the plain suture group had a mean % alteration of 68.39 (Std. Dev=10.44, Std. Error of Mean=2.395, n=19).

FIG. 24 is a dot plot showing the % alterations in a Y maze in mice with an absorbable suture versus a plain suture, with and without treatment according to some embodiments. Measurement of the number of alternations within the 3 arms of a Y-Maze according to the following formula: % alternation=(total alternations/(total entries−2)×100). The untreated absorbable suture group had a mean % alteration of 61.907 (SEM=7.246, n=6), and the untreated plain suture group had a mean % alteration of 65.130 (SEM=4.682, n=7). The treated absorbable suture group had a mean % alteration of 61.396 (SEM=3.556, n=7), and the treated plain suture group had a mean % alteration of 70.296 (SEM=2.644, n=12).

FIG. 25 is a schematic showing the treatment protocol according to some embodiments.

FIG. 26A is a dot plot showing Evan's blue data in the right hemisphere of mice treated with fingolimod.

FIG. 26B is a normal QQ plot showing results of testing normality and Log normality of Evan's Blue data.

FIG. 27 is a dot plot showing region-of-interest (ROI) Evan's Blue data from individual mice (*p<0.05).

FIG. 28 is a dot plot showing region-of-interest (ROI) Evan's Blue data from individual mice.

FIG. 29A is a graph showing peak velocity based on Evan's Blue Data in mice without treatment (placebo).

FIG. 29B is a graph showing peak velocity based on Evan's Blue Data in mice with fingolimod treatment.

FIG. 30A is a bar graph showing expression levels of S1P1 as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)). Abbreviations for FIG. 30A to FIG. 38B: RH, right (ipsilateral) hemisphere, n=3; LH, left (contralateral) hemisphere (n=3 mice in each group); No TT=No treatment (vehicle); TT=Treatment (fingolimod, 0.01 mg/day in drinking water, i.e. 0.3 mg/kg/d for the two last weeks.

FIG. 30B is a bar graph showing expression levels of S1P1 as measured by RT-PCR in the in the microvascular fraction of the whole brain (not treated or treated, n=5 per group, unpaired t-test (no TT vs. TT)).

FIG. 31A is a bar graph showing expression levels of S1P3 as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 31B is a bar graph showing expression levels of S1P3 as measured by RT-PCR in the in the microvascular fraction of the whole brain (not treated or treated, n=5 per group, unpaired t-test (no TT vs. TT)).

FIG. 32A is a bar graph showing expression levels of B2M as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 32 B is a bar graph showing expression levels of B2M as measured by RT-PCR in the in the microvascular fraction of the whole brain (not treated or treated, n=5 per group, unpaired t-test (no TT vs. TT)).

FIG. 33A is a bar graph showing expression levels of CERS4 as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 33 B is a bar graph showing expression levels of CERS4 as measured by RT-PCR in the in the microvascular fraction of the whole brain (not treated or treated, n=5 per group, unpaired t-test (no TT vs. TT)).

FIG. 34A is a bar graph showing expression levels of CERS2 as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 34 B is a bar graph showing expression levels of CERS2 as measured by RT-PCR in the in the microvascular fraction of the whole brain (not treated or treated, n=5 per group, unpaired t-test (no TT vs. TT)).

FIG. 35A is a bar graph showing expression levels of Claudine 5 as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 35 B is a bar graph showing expression levels of Claudine 5 as measured by RT-PCR in the in the microvascular fraction of the whole brain (not treated or treated, n=5 per group, unpaired t-test (no TT vs. TT)).

FIG. 36A is a bar graph showing expression levels of ICAM as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 36 B is a bar graph showing expression levels of ICAM as measured by RT-PCR in the in the microvascular fraction of the whole brain (not treated or treated, n=5 per group, unpaired t-test (no TT vs. TT)).

FIG. 37A is a bar graph showing expression levels of VCAM as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 37 B is a bar graph showing expression levels of VCAM as measured by RT-PCR in the in the microvascular fraction of the whole brain (not treated or treated, n=5 per group, unpaired t-test (no TT vs. TT)).

FIG. 38A is a bar graph showing expression levels of MMP9 as measured by RT-PCR in the ipsi- and contra-lateral hemispheres of the brain of three (3) mice treated of not treated (paired statistical analysis, left hemisphere (LH) vs. right hemisphere (RH), unpaired t-test (no TT vs. TT)).

FIG. 38 B is a bar graph showing expression levels of MMP9 as measured by RT-PCR in the in the microvascular fraction of the whole brain (not treated or treated, n=5 per group, unpaired t-test (no TT vs. TT)).

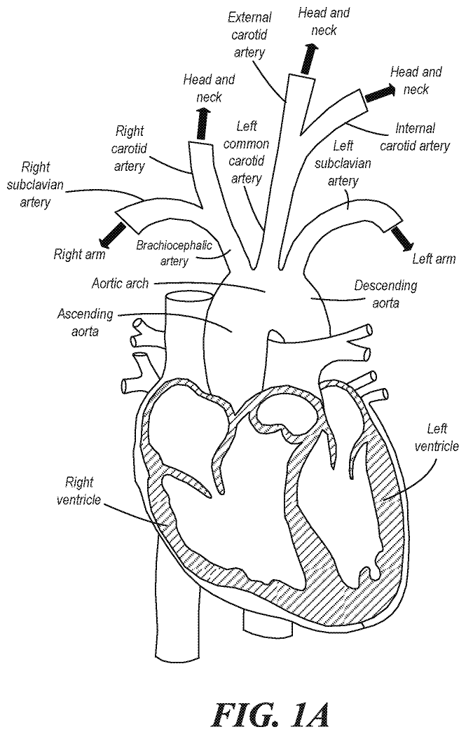

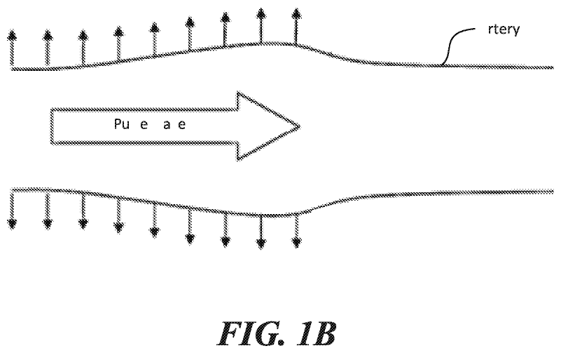

### DETAILED DESCRIPTION

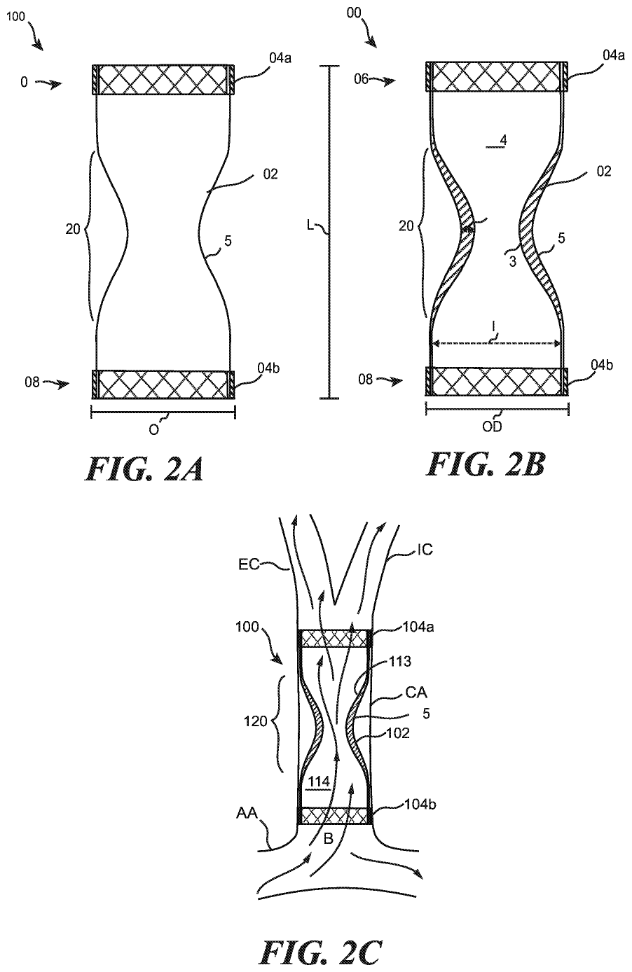

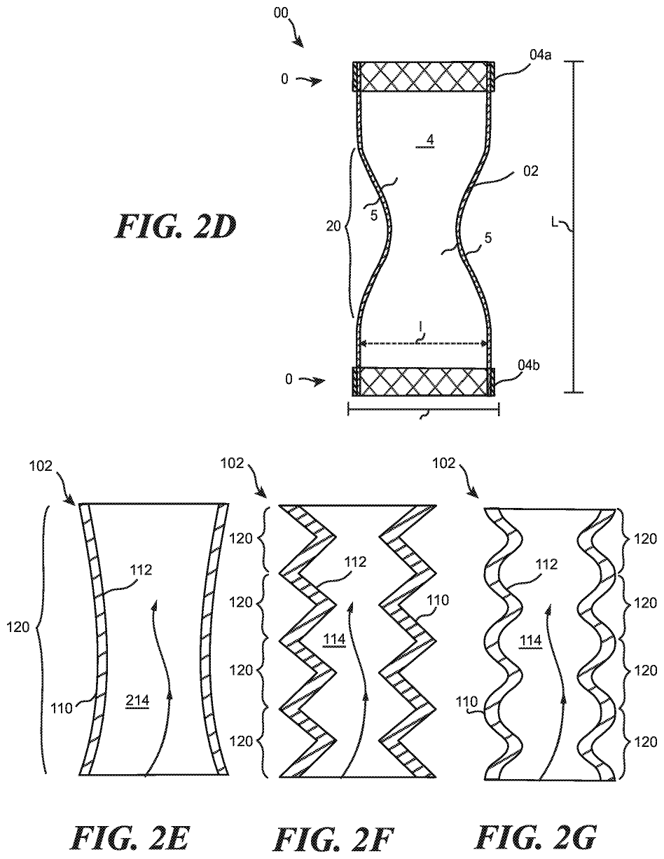

The present technology is directed to combinatorial therapies including an implantable damping device and a therapeutic agent (e.g., a EDG Receptor Family modulators, MMP inhibitors, and senolytic agents) for treating and/or preventing the progression of a condition, including neurological conditions such as dementia (e.g., vascular dementia and age-related dementia), and associated systems and methods of use. Some embodiments of the present technology, for example, are directed to combinatorial device and therapies including damping devices having an anchoring member and a flexible, compliant damping member having an outer surface and an inner surface defining a lumen configured to direct blood flow. The inner surface is configured such that a cross-sectional dimension of the lumen varies. For example, the outer surface and the inner surface can be separated from each other by a distance that varies along the length of the damping member. The damping member can further include a first end portion, a second end portion opposite the first end portion, and a damping region between the first and second end portions. The distance between the outer surface and the inner surface of the damping member can be greater at the damping region than at either of the first or second end portions. When blood flows through the damping member during systole, the damping member absorbs a portion of the pulsatile energy of the blood to reduce the magnitude of the pulse pressure transmitted to a portion of the blood vessel distal to the damping device. Additional embodiments of the present technology, for example, are directed to combinatorial device and therapies including therapeutic agents (e.g., EDG Receptor Family modulators, MMP inhibitors, and senolytic agents) that have been developed or are currently being developed to treat or otherwise slow the effects of neurological conditions. These therapeutic agents, and other therapeutic agents derived from and/or otherwise based upon these therapeutic agents, are included in embodiments of the present technology. Specific details of several embodiments of the technology are described below with reference to FIGS. 2A-20.

With regard to the terms “distal” and “proximal” within this description, unless otherwise specified, the terms can reference a relative position of the portions of a damping device and/or an associated delivery device with reference to an operator, direction of blood flow through a vessel, and/or a location in the vasculature. For example, in referring to a delivery catheter suitable to deliver and position various damping devices described herein, “proximal” refers to a position closer to the operator of the device or an incision into the vasculature, and “distal” refers to a position that is more distant from the operator of the device or further from the incision along the vasculature (e.g., the end of the catheter).

As used herein, “artery” and “arteries that supply blood to the brain,” include any arterial blood vessel (or portion thereof) that provides oxygenated blood to the brain. For example, “arteries” or “arteries that supply blood to the brain” can include the ascending aorta, the aortic arch, the brachiocephalic trunk, the right common carotid artery, the left common carotid artery, the left and right internal carotid arteries, the left and right external carotid arteries, and/or any branch and/or extension of any of the arterial vessels described above.

As used herein, the terms “therapeutic agent”, “agent”, “therapy”, and “therapies” are used interchangeably within this description to refer to drugs and/or therapies described herein, including EDG Receptor Family Modulators, an MMP inhibitors, and/or a senolytic agents. Agents and therapies may be composed of a wide variety of substances including, but not limited to, sugars (or other small molecules), synthetic chemicals, proteins or peptides, nucleic acids, complex combinations of those substances, or may be living entities such as cells or tissues. Agents and therapies include a wide variety of products and treatments that may be isolated from or otherwise derived from natural sources (e.g., human, animal, plant, microorganism) using recombinant, synthetic, or other biotechnology methods and technologies. For example, a peptide or other natural molecule that is synthetically produced may be considered an agent in accordance with this disclosure.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

With regard to the term “neurological condition” within this description, unless otherwise specified, the term refers to a condition, a disorder, and/or a disease of the brain, spine, and nerves connecting the brain and the spine. Neurological conditions include, but are not limited to dementia (e.g., vascular, frontotemporal, Lewy body), Alzheimer's disease, Huntington's disease, cognitive impairment, Parkinson's disease, neuralgia, tumor, cancer, stroke, aneurysm, epilepsy, headache, and/or migraine.

A “subject in need thereof” as used herein refers to a mammalian subject, preferably a human, who has been diagnosed with a neurologic condition, is suspected of having a neurologic condition, and/or exhibits one or more symptoms or risk factors associated with a neurologic condition.

The terms “treating” and “treatment” in relation to a given condition, disease, or disorder are used interchangeably and include, but are not limited to, inhibiting the disease or disorder, for example, arresting the development or rate of development of the condition, disease, or disorder; relieving the condition, disease, or disorder, for example, causing regression of the condition, disease, or disorder; or relieving a condition caused by or resulting from the disease or disorder, for example, arresting, relieving, preventing, or causing regression of at least one of the symptoms of the disease or disorder.

The terms “preventing” and “prevention” in relation to a given condition, disease, or disorder are used interchangeably and include, but are not limited to, preventing or delaying the onset of its development if none had occurred; preventing or delaying the condition, disease, or disorder from occurring in a subject that may be predisposed to the condition, disease, or disorder but has not yet been diagnosed as having the condition, disease; or disorder, and/or preventing or delaying further development of the condition, disease, or disorder if already present.

As used herein, “route” in relation to administration of one or more therapies, such as a therapeutic agent (e.g., drug), refers to a path by which the therapeutic agent is delivered to a subject, for example, a subject's body. A route of therapeutic administration include enteral and parenteral routes of administration. Enteral administration includes oral, rectal, intestinal, and/or enema. Parenteral includes topical, transdermal, epidural, intracerebral, intracerebroventricular, epicutaneous, sublingual, sublabial, buccal, inhalational (e.g., nasal), intravenous, intraarticular, intracardiac, intradermal, intramuscular, intraocular, intraosseous infusion, intraperitoneal, intrathecal, intravitreal, subcutaneous, perivascular, implantation, vaginal, otic, and/or transmucosal.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein is to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include, but not be limited to, systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense that one having skill in the art would understand the convention (e.g., “a system having at least one of A, B, or C” would include, but not be limited to, systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting. Further, headings provided herein are for convenience only and do not interpret the scope or meaning of the claimed embodiments.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

While the present invention is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

### I. SELECTED INTRAVASCULAR EMBODIMENTS OF DAMPING DEVICES

FIGS. 2A and 2B are a front view and a front cross-sectional view, respectively, of a damping device 100 configured in accordance with the present technology shown in an expanded or deployed state. FIG. 2C is a front view of the damping device 100 in a deployed state positioned in a carotid artery CA (e.g., the left or right carotid artery). Referring to FIGS. 2A-2C together, the damping device 100 includes a flexible, viscoelastic damping member 102 (e.g., a cushioning member) and anchoring members 104 (identified individually as first and second anchoring members 104a and 104b, respectively). The damping member 102 includes an undulating or hourglass-shaped sidewall having an outer surface 115 and an inner surface 113 (FIGS. 2B and 2C) that defines a lumen 114 configured to receive blood flow therethrough. The outer surface 115 is separated from the inner surface 113 by a distance t (FIG. 2B). The damping member 102 has a length L, a first end portion 106, and a second end portion 108 opposite the first end portion 106 along its length L, and a damping region 120 between the first end portion 106 and the second end portion 108. In the embodiment shown in FIGS. 2A-2C, the distance t between the outer and inner surfaces 115 and 113 varies along the length L of the damping member 102 when it is in a deployed, relaxed state. In some embodiments, the distance t between the outer and inner surfaces 115 and 113, on average, can be greater at the damping region 120 than at either of the first or second end portions 106, 108. In other embodiments, the damping member 102 can have other suitable shapes (for example, FIGS. 2E-2G), sizes, and/or configurations. For example, as shown in FIG. 2D, the distance t between the outer and inner surfaces 115 and 113 may be generally constant along the length of the damping member 102 and/or the damping region 120 when the damping member 102 is in a deployed, relaxed state.

The damping member 102 shown in FIGS. 2A-2C is a solid piece of material that is molded, extruded, or otherwise formed into the desired shape. The damping member 102 can be made of a biocompatible, compliant, viscoelastic material that is configured to deform in response to local fluid pressure in the artery. As the damping member 102 deforms, the damping member 102 absorbs a portion of the pulse pressure. The damping member 102, for example, can be made of a biocompatible synthetic elastomer, such as silicone rubber (VMQ), Tufel I and Tufel III elastomers (GE Advanced Materials, Pittsfield, MA), Sorbothane® (Sorbothane, Incorporated, Kent, OH), and others. The damping member 102 can be flexible and elastic such that the inner diameter ID of the damping member 102 at the damping region 120 increases as a systolic pressure wave propagates through the damping region 120. For example, a systolic pressure wave may push the inner surface 113 radially outwardly, thus forcing a portion of the outer surface 115 to also deform radially outwardly. Additionally, the damping member 102 can also optionally be compressible such that the distance t between the inner and outer surfaces 115 and 113 decreases to further open the inner diameter ID of the damping region 120 as the systolic pressure wave engages the damping region 120. For example, a systolic pressure wave may push the inner surface 113 radially outwardly while the contour of the outer surface 115 remains generally unaffected.

In the embodiment shown in FIGS. 2A-2C, the anchoring members 104a-104b individually comprise a generally cylindrical structure configured to expand from a low-profile state to a deployed state in apposition with the blood vessel wall. Each of the anchoring members 104a-b can be a stent formed from a laser cut metal, such as a superelastic material (e.g., Nitinol) or stainless steel. All or a portion of each of the anchoring members can include a radiopaque coating to improve visualization of the device during delivery, and/or the anchoring members may include one or more radiopaque markers. In other embodiments, the individual anchoring members 104a-104b can comprise a mesh or woven (e.g., a braid) construction in addition to or in place of a laser cut stent. For example, the individual anchoring members 104a-104b can include a tube or braided mesh formed from a plurality of flexible wires or filaments arranged in a diamond pattern or other configuration. In some embodiments, all or a portion of one or both of the anchoring members 104a-104b can be covered by a graft material (such as Dacron) to promote sealing with the vessel wall. Additionally, all or a portion of one or both anchoring members can include one or more biomaterials.

In the embodiment shown in FIGS. 2A-2B, the anchoring members 104a-104b are positioned around the damping member 102 at the first and second end portions 106, 108, respectively. As such, in this embodiment, the outer diameter OD of the damping member 102 is less than the inner diameter of the anchoring members 104a-104b. Also in the embodiment shown in FIGS. 2A-2B, the anchoring members 104a-104b are positioned around the damping member 102 only at the first and second end portions 106, 108, respectively. As such, in several embodiments of the present technology, the damping region 120 of the damping member 120 is not surrounded by a stent-like structure or braided material. In other embodiments, the anchoring members 104 and damping member 102 may have other suitable configurations. For example, the anchoring members 104a-104b may be positioned at other locations along the length L of the damping member 102, though not along the full length of the damping member 102. Also, in some embodiments, all or a portion of one or both anchoring members 104a-104b may be positioned radially outwardly of all or a portion of the damping member 102. Although the damping device 100 shown in FIGS. 2A-2B includes two anchoring members 104a-104b, in other embodiments the damping device 100 can have more or fewer anchoring members (e.g., one anchoring member, three anchoring members, four anchoring members, etc.).

In some embodiments, a biocompatible gel or liquid may be located between the wall of the artery A and the outer surface 115 of the damping member 102 to prevent the ingression of blood into the void defined between the first anchoring member 104a, the second anchoring member 104b, the damping member 102, and the inner wall of the artery CA. Alternatively, air or another gas may be located between the internal wall of the carotid artery CA and the damping member 102 to prevent the ingression of blood into the void.

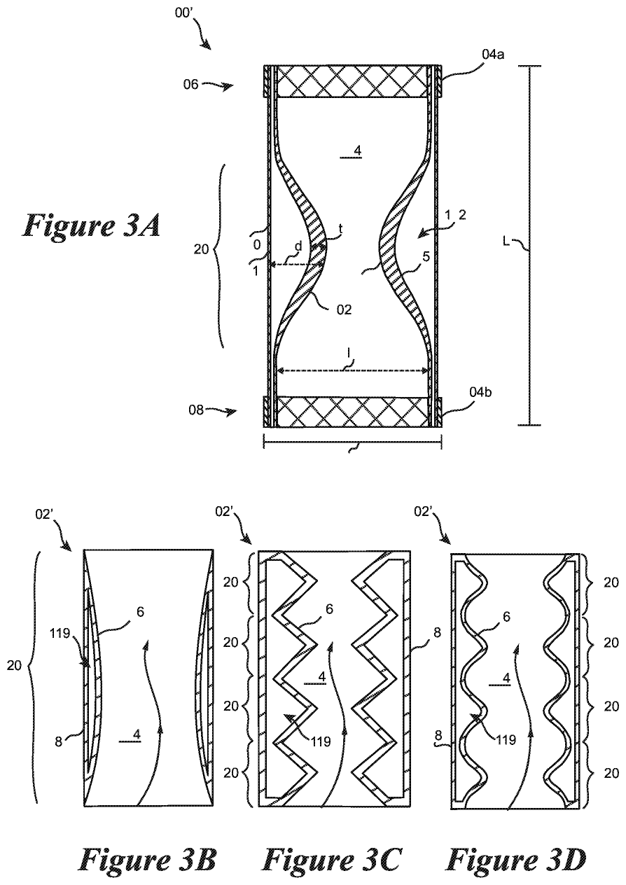

FIG. 3A is a front cross-sectional view of another embodiment of a damping device 100′ in accordance with the present technology. The embodiment of the damping device 100′ shown in FIG. 3A is similar to the embodiment of the damping device 100 shown in FIGS. 2A-2C, and like reference numbers refer to like components in FIGS. 2A-2C and FIG. 3A. As shown in FIG. 3A, the damping device 100′ includes an inner damping member 102 and an outer layer 130 surrounding the damping member 102. The outer layer 130 has an outer surface 131 and, in the embodiment shown in FIG. 3A, the first and second anchoring members 104a-b are attached to the outer surface 131. At least along the damping region 120, the outer layer 130 is spaced apart from the outer surface 115 of the damping member 102 to form a chamber 132. The chamber 132 can be at least partially filled with a fluid, such as a gas, liquid, or gel. The device 100′ has a length L and a distance d between the outer surface 131 of the outer layer 130 and the inner surface 113 of the damping member 102. Along the damping region 120, the distance d between the outer and inner surfaces 131 and 113 increases then decreases in a radial direction when the damping member 102 is in a deployed, relaxed state. On average, the distance d between the outer surface 131 and the inner surface 113 of the damping member 102 is greater at the damping region 120 than at either of the first or second end portions 106, 108. As a result, the diameter ID of the lumen 114 varies along the length L. For example, the outer surface 131 and/or the outer layer 130 can be generally cylindrical in an unbiased state, and the inner surface 113 and/or the damping member 102 can have an undulating or hourglass shape. In other embodiments, the outer surface 131 and/or the outer layer 130 can be other suitable shapes, and the inner surface 113 and/or the damping member 102 can be other suitable shapes (FIGS. 3B-3D).

In some embodiments, instead of the damping device 100′ having a separate outer layer 130, the damping member 102 can be molded, formed, or otherwise extruded to enclose a cavity. For example, as shown in FIGS. 3B-3D, the damping member 102′ can include an inner layer 116, an outer layer 118, and a cavity 119 therebetween. The cavity 119 can be at least partially filled with a fluid, such as a gas, liquid, or gel.

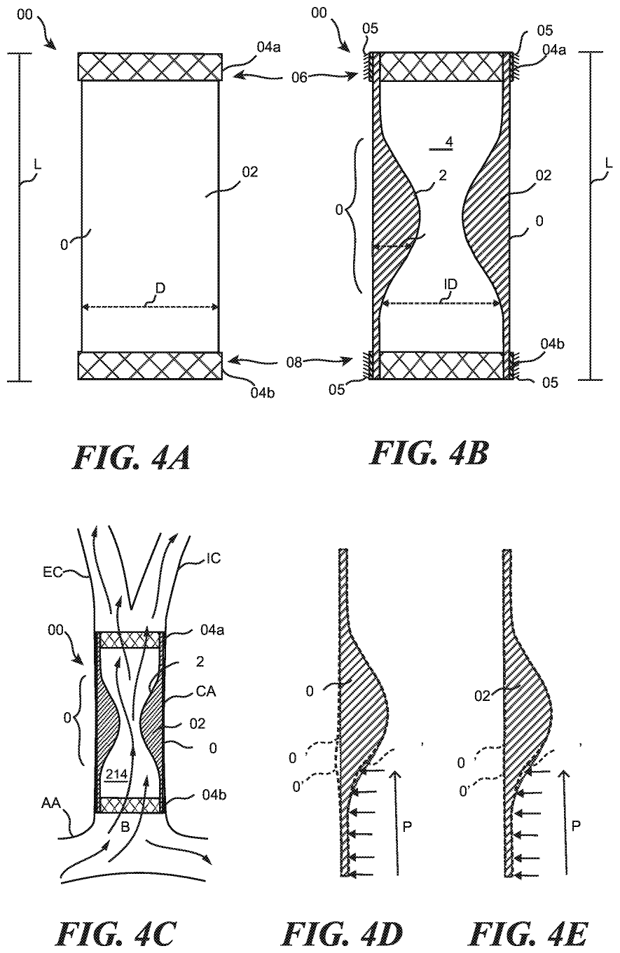

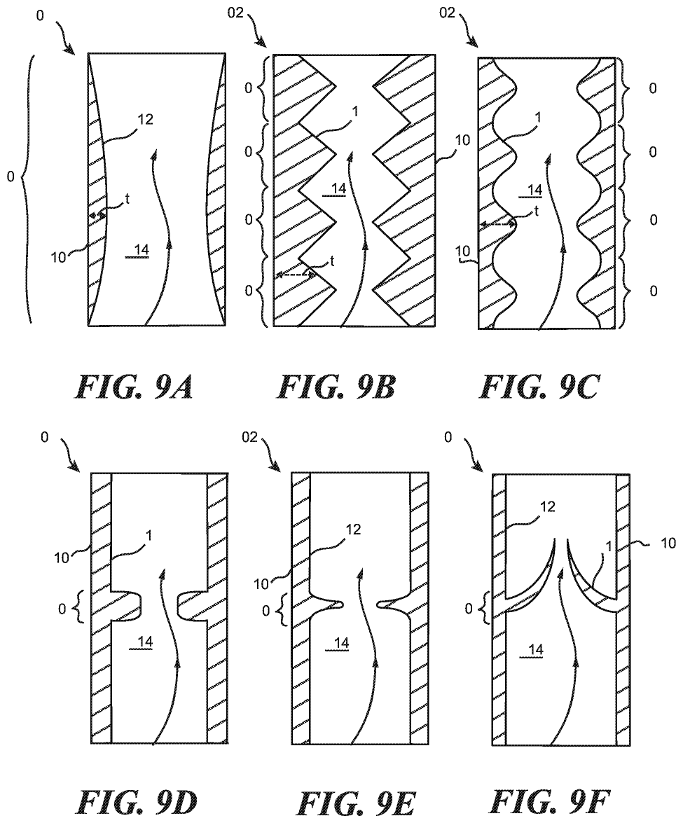

FIGS. 4A and 4B are a front view and a front cross-sectional view, respectively, of another embodiment of a damping device 200 configured in accordance with the present technology shown in an expanded or deployed state. FIG. 4C is a front cross-sectional view of the damping device 200 in a deployed state positioned in a carotid artery (e.g., the left or right carotid artery). Referring to FIGS. 4A-4C together, the damping device 200 includes a flexible, viscoelastic damping member 202 (e.g., a cushioning member) and anchoring members 204 (identified individually as first and second anchoring members 204a-204b, respectively). As shown in FIGS. 4B and 4C, the damping member 202 includes a generally tubular sidewall having a cylindrical outer surface 210 and an inner surface 212 that defines a lumen 214 configured to receive blood flow therethrough. The outer surface 210 is separated from the inner surface 212 by a distance t (FIG. 4B). The damping member 202 has a length L, a first end portion 206, and a second end portion 208 opposite the first end portion 206 along its length L, and a damping region 220 between the first end portion 206 and the second end portion 208. Along the damping region 220, the distance t between the outer and inner surfaces 210 and 212 increases then decreases in a radial direction when the damping member 202 is in a deployed, relaxed state. On average, the distance t between the outer and inner surfaces 210 and 212 of the damping member 202 is greater at the damping region 220 than at either of the first or second end portions 206, 208. As a result, the inner diameter ID of the damping member 202 varies along its length L relative to the outer diameter OD of the damping member 202. For example, the outer surface 210 can be generally cylindrical in an unbiased state, and the inner surface 212 can have an undulating or hourglass shape. As described in greater detail below with respect to FIGS. 9A-9F, the damping member 202 can have other suitable shapes, sizes, and/or configurations.

The damping member 202 shown in FIGS. 4A-4C is a solid piece of material that is molded, extruded, or otherwise formed into the desired shape. The damping member 202 can be made of a biocompatible, compliant, viscoelastic material that is configured to deform in response to local fluid pressure in the artery. As the damping member 202 deforms, the damping member 202 absorbs a portion of the pulse pressure. The damping member 202, for example, can be made of a biocompatible synthetic elastomer, such as silicone rubber (VMQ), Tufel I and Tufel III elastomers (GE Advanced Materials, Pittsfield, MA), Sorbothane® (Sorbothane, Incorporated, Kent, OH), and others. The damping member 202 can be flexible and elastic such that the inner diameter ID of the damping member 202 at the damping region 220 increases as a systolic pressure wave P (FIG. 4D) propagates through the damping region 220. For example, as shown schematically in the isolated, cross-sectional view of a portion of a damping member 202 before and during deformation (damping member 202′, shown in dashed lines) in FIG. 4D, the systolic pressure wave P may push the inner surface 212′ radially outwardly, thus forcing a portion of the outer surface 210′ to also deform radially outwardly. Additionally, the damping member 202 can also optionally be compressible such that the distance t between the inner and outer surfaces 210 and 212 decreases to further open the inner diameter ID of the damping region 220 as the systolic pressure wave P engages the damping region 220. For example, as shown schematically in the isolated, cross-sectional view of a portion of a damping member 202 before and during deformation (damping member 202′, shown in dashed lines) in FIG. 4E, the systolic pressure wave P may push the inner surface 212′ radially outwardly while the contour of the outer surface 210′ remains generally unaffected.

In the embodiment shown in FIGS. 4A-4C, the anchoring members 204a-204b individually comprise a generally cylindrical structure configured to expand from a low-profile state to a deployed state in apposition with the blood vessel wall. Each of the anchoring members 204a-b can be a stent formed from a laser cut metal, such as a superelastic material (e.g., Nitinol) or stainless steel. All or a portion of each of the anchoring members can include a radiopaque coating to improve visualization of the device during delivery, and/or the anchoring members may include one or more radiopaque markers. In other embodiments, the individual anchoring members 204a-204b can comprise a mesh or woven (e.g., a braid) construction in addition to or in place of a laser cut stent. For example, the individual anchoring members 204a-204b can include a tube or braided mesh formed from a plurality of flexible wires or filaments arranged in a diamond pattern or other configuration. In some embodiments, all or a portion of one or both of the anchoring members 204a-204b can be covered by a graft material (such as Dacron) to promote sealing with the vessel wall.

In the embodiment shown in FIGS. 4A-4B, the anchoring members 204a-204b are positioned around the damping member 202 at the first and second end portions 206, 208, respectively. As such, in this embodiment, the outer diameter OD (FIG. 4A) of the damping member 202 is less than the inner diameter of the anchoring members 204a-204b. Also in the embodiment shown in FIGS. 4A-4B, the anchoring members 204a-204b are positioned around the damping member 202 only at the first and second end portions 206, 208, respectively. As such, in several embodiments of the present technology, the damping region 220 of the damping member 220 is not surrounded by a stent-like structure or braided material. In other embodiments, the anchoring members 204a-204b and damping member 202 may have other suitable configurations. For example, the anchoring members 204a-204b may be positioned at other locations along the length L of the damping member 202, though not along the full length of the damping member 202. Also, in some embodiments, all or a portion of one or both anchoring members 204a-204b may be positioned radially outwardly of all or a portion of the damping member 202. Although the damping device 200 shown in FIGS. 4A-4B includes two anchoring members 204a-204b, in other embodiments the damping device 200 can have more or fewer anchoring members (e.g., one anchoring member, three anchoring members, four anchoring members, etc.).

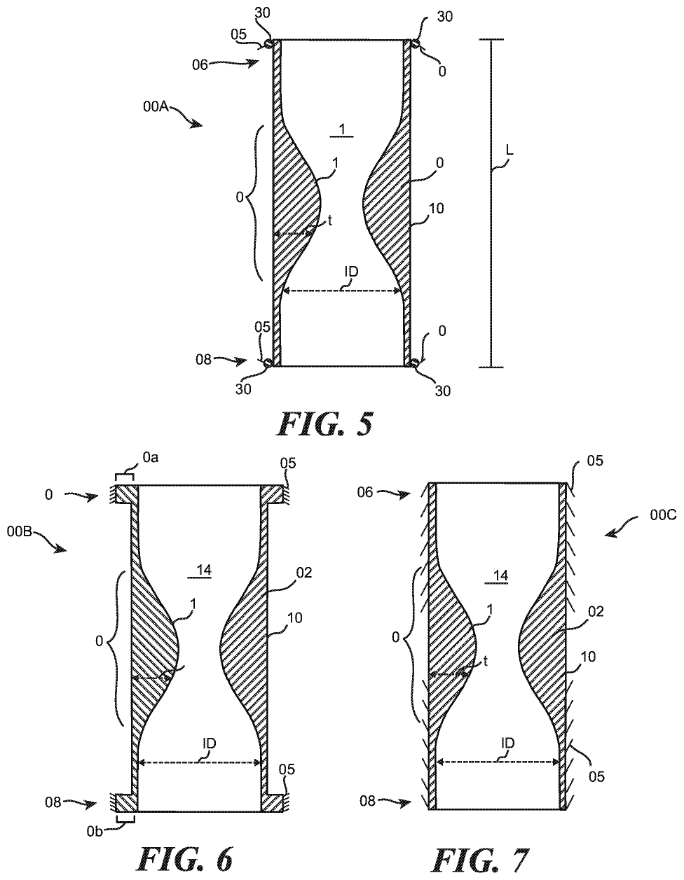

In some embodiments, one or both of the anchoring members 204a-204b can optionally include one or more fixation elements 205 (FIG. 4B) configured to engage the blood vessel wall. The fixation elements 205 can include, for example, one or more hooks or barbs that, in the deployed state, extend outwardly away from the corresponding frames of the anchoring member 204a-204b to penetrate the vessel wall at the treatment site. In these and other embodiments, one or more of the fixation elements can be atraumatic. Additionally, referring to the damping device 200A shown in FIG. 5, in certain embodiments the damping device 200 may not include a stent-type or braid-type anchoring member, but rather the frame of the anchoring members 204 can be one or more expandable rings 230. For example, in some embodiments the damping device 200 can include two rings 230, each attached to a respective end portion 206 and 208, and the plurality of fixation elements 205 can extend outwardly from the rings 230. In still other embodiments, such as the damping device 200B shown in FIG. 6, the anchoring members 204 can be integral portions of the end portions 206, 208, such as thick wall portions 240a-b of the damping member 202 that extend radially outward from the outer wall of the damping region 220, instead of separate metal or polymeric components. In this embodiment, the fixation elements 205 can extend outwardly from integral anchoring members 240a-b at the first and second end portions 206, 208 of the damping member 202. When the damping device 200 is in a deployed state, the fixation elements 205 extend outwardly away from the outer surface of the damping member 202 to engage vessel wall tissue. In yet other embodiments, the fixation elements 205 can extend outwardly from the outer surface 210 of the damping member 202, as shown in the damping device 200C of FIG. 7.

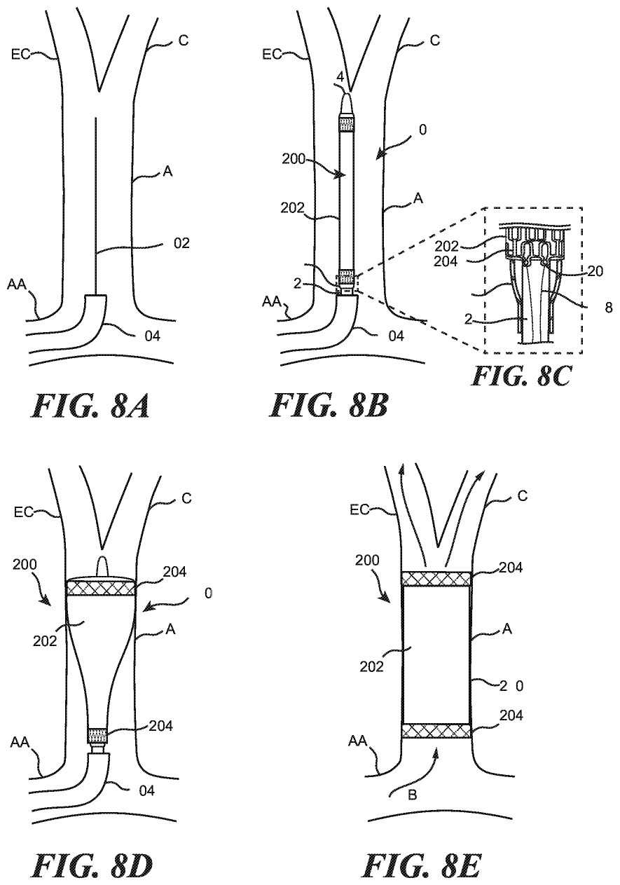

FIGS. 8A-8E illustrate a method for positioning a damping device of the present disclosure at a treatment location within an artery A (such as the left and/or right common carotid artery CA). Although FIGS. 8B-8E depict the damping device 200 shown in FIGS. 4A and 4B, the methods and systems described with respect to FIGS. 8A-8E can be utilized for any of the damping devices 100, 100′, 200, 200A, 200B, and 200C described with respect to FIGS. 2A-7 and FIGS. 9A-9F.

As shown in FIG. 8A, a guidewire 602 may first be advanced intravascularly to the treatment site from an access site, such as a femoral or a radial artery. A guide catheter 604 may then be advanced along the guidewire 602 until at least a distal portion of the guide catheter 604 is positioned at the treatment site. In these and other embodiments, a rapid-exchange technique may be utilized. In some embodiments, the guide catheter 604 may have a pre-shaped or steerable distal end portion to direct the guide catheter 604 through one or more bends in the vasculature. For example, the guide catheter 604 shown in FIGS. 8A-8E has a curved distal end portion configured to navigate through the ascending aorta AA and preferentially bend or flex at the left and/or right common carotid artery A to direct the guide catheter 604 into the artery A.

Image guidance, e.g., computed tomography (CT), fluoroscopy, angiography, intravascular ultrasound (IVUS), optical coherence tomography (OCT), or another suitable guidance modality, or combinations thereof, may be used to aid the clinician's positioning and manipulation of the damping device 200. For example, a fluoroscopy system (e.g., including a flat-panel detector, x-ray, or c-arm) can be rotated to accurately visualize and identify the target treatment site. In other embodiments, the treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering the damping device 200. Further, in some embodiments, image guidance components (e.g., IVUS, OCT) may be integrated with the delivery catheter and/or run in parallel with the delivery catheter to provide image guidance during positioning of the damping device 200.

Once the guide catheter 604 is positioned at the treatment site, the guidewire 602 may be withdrawn. As shown in FIGS. 8B and 8C, a delivery assembly 610 carrying the damping device 200 may then be advanced distally through the guide catheter 604 to the treatment site. In some embodiments, the delivery assembly 610 includes an elongated shaft 612 having an atraumatic distal tip 614 (FIG. 8B) and an expandable member 616 (e.g., an inflatable balloon, an expandable cage, etc.) positioned around a distal portion of the elongated shaft 612. The damping device 200 can be positioned around the expandable member 616. As shown in FIG. 8D, expansion or inflation of the expandable member 616 forces at least a portion of the damping device 200 radially outwardly into contact with the arterial wall. In some embodiments, the delivery assembly 610 can include a distal expandable member for deploying a distal portion of the damping device 200, and a proximal expandable member for deploying a proximal portion of the damping device 200. In other embodiments, the entire length of the damping device 200 may be expanded at the same time by deploying one or more expandable members.

In some procedures the clinician may want to stretch or elongate the damping device 200 before deploying the proximal second anchoring member 204b against the arterial wall. To address this need, the delivery assembly 610 and/or damping device 200 can optionally include a tensioning mechanism for pulling or providing a tensile stress on the second anchoring member 204b, thereby increasing the length of the damping member 202 and/or a distance between the first and second anchoring members 204a, 204b. For example, as shown in FIG. 8C, the second anchoring member 204b can include one or more coupling portions 205 (e.g., one or more eyelets extending proximally from the anchoring frame) and one or more coupling members 618 (e.g., a suture, a thread, a filament, a tether, etc.) extending between the second anchoring member 204b and a proximal portion (not shown) of the delivery assembly 610 (e.g., a handle). The coupling members 618 are configured to releasably engage the coupling portions 205 to mechanically couple the second anchoring member 204b to a proximal portion of the delivery assembly 610. A clinician can apply a tensile force to the coupling member 618 to elongate the damping device 200 and/or damping member 202 and adjust the longitudinal position of the second anchoring member 204b. Once the second anchoring member 204b is positioned at a desired longitudinal position relative to the first anchoring member 204a and/or the local anatomy, the second anchoring member 204b can be expanded into contact with the arterial wall (e.g., via deployment of one or more expandable members). Before, during, and/or after expansion of the second anchoring member 204b, the coupling member(s) 618 may be disengaged from the second anchoring member 204b. For example, in some embodiments, the operator can force the coupling members 618 to break along their lengths by applying a tensile force that is less than a force that would be required to dislodge one or both of the first and second anchoring members 204a, 204b. Once disengaged from the second anchoring member 204b and/or the damping device 200, the coupling member(s) 618 can then be withdrawn from the treatment site through the guide catheter 604.

In other embodiments, other tensioning mechanisms may be utilized. For example, in some embodiments, the damping device 200 includes a releasable clasp, ring, or hook which is selectively releasable by the operator. The clasp, ring or hook may be any type that permits securement of the thread to the second anchoring member 204b, and which can be selectively opened or released to disengage the thread from the second anchoring member 204b. The releasing can be controlled by the clinician from an extracorporeal location. Although the tensioning mechanism is described herein with respect to the second anchoring member 204b, it will be appreciated that other portions of the damping device 200 and/or the delivery assembly 610 (such as the first anchoring member 204a) can be coupled to a tensioning mechanism.

In certain embodiments, the damping member 202 and/or individual anchoring members 204a, 204b may be self-expanding. For example, the delivery assembly 610 can include a delivery sheath (not shown) that surrounds and radially constrains the damping device 200 during delivery to the treatment site. Upon reaching the treatment site, the delivery sheath may be at least partially withdrawn or retracted to allow the damping member 202 and/or the individual anchoring members 204a, 204b to expand. In some embodiments, expansion of the anchoring members 204 may drive expansion of the damping member 202. For example, the anchoring members 204 may be fixedly attached to the damping member 202, and expansion of one or both anchoring 204 pulls or pushes (depending on the relative positioning of the damping member 202 and anchoring members 204) the damping member 202 radially outwardly.

As best shown in FIG. 8C, once the damping device 200 is positioned at the treatment site (e.g., in a left or right common carotid artery), oxygenated blood ejected from the left ventricle flows through the lumen 214 of the damping member 202. As the blood contacts the damping region 220 of the damping member 202, the damping region 220 deforms to absorb a portion of the pulsatile energy of the blood, which reduces a magnitude of a pulse pressure transmitted to the portions of the artery distal to the damping device 200 (such as the more-sensitive cerebral arteries). The damping region 202 acts a pressure limiter that distributes the pressure of the systolic phase of the cardiac cycle more evenly downstream from the damping device 200 without unduly compromising the volume of blood flow through the damping device 200. Accordingly, the damping device 200 reduces the pulsatile stress on downstream portions of the arterial network to prevent or at least partially reduce the manifestations of vascular dementia and/or age-related dementia.

In some procedures, it may be beneficial to deliver multiple damping devices 200 to multiple arterial locations. For example, after deploying a first damping device 200 at a first arterial location (e.g., the left or right common carotid artery, an internal or external carotid artery, the ascending aorta, etc.), the clinician may then position and deploy a second damping device 200 at a second arterial location different than the first arterial location (e.g., the left or right common carotid artery, an internal or external carotid artery, the ascending aorta etc.). In a particular application, a first damping device is deployed in the left common carotid artery and the second damping device is deployed in the right common carotid artery. In other embodiments, two or more damping devices 200 may be delivered simultaneously.

In some embodiments, an additional stent of larger diameter may be placed within the vessel prior to deployment of the damping device 200 to expand the diameter of the vessel in preparation for the device. Subsequently, the damping device 200 can be deployed within the larger stent. This may assist to reduce impact on the residual diameter of the vessel, and thereby reduce impact on blood flow rate.

FIGS. 9A-9F are schematic cross-sectional views of several embodiments of damping members in accordance with the present technology. Like reference numbers refer to similar or identical components in FIGS. 2A-9F. In the embodiment shown in FIG. 9A, the inner surface 212 of the damping member 202 is curved along its entire length. The distance between the outer surface 210 and the inner surface 212 gradually increases then decreases in a distal direction. As such, the damping region 220 extends the entire length of the damping member 202. FIGS. 9B and 9C illustrate embodiments of the damping member 202 in which the inner surface 212 has a series of damping regions 220 defined by undulations in the inner surface 212. In these embodiments, the distance t increases, then decreases, then increases, then decreases, etc. in a distal direction. In FIG. 9B, the damping regions 220 are generally linear, while in FIG. 9C, the damping regions 220 are generally curved. FIGS. 9D-9E illustrate embodiments of damping members 202 having damping regions 220 comprising an annular ring projecting radially inwardly into the lumen 214. One or more portions of the annular ring may flex in a longitudinal direction in response to blood flow. As shown in FIG. 9F, in some embodiments the damping member 220 can comprise two or more opposing leaflets 221.

### II. SELECTED RESECTION EMBODIMENTS OF DAMPING DEVICES

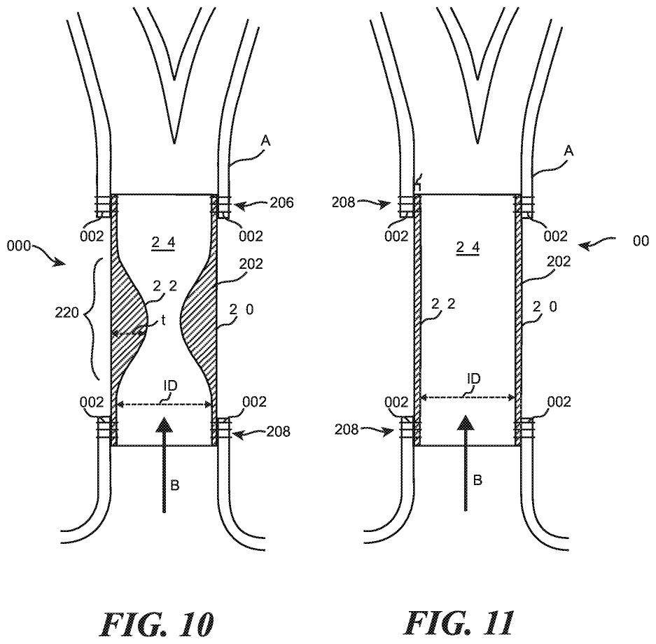

FIGS. 10 and 11 are schematic cross-sectional views of several embodiments of damping devices in accordance with the present technology. Like reference numbers refer to similar or identical components in FIGS. 2A-15. FIG. 10, for example, shows a damping device 1000 comprising only the damping member 202. A portion of the arterial wall A may be resected, and the damping member 202 may be coupled to the open ends of the resected artery (e.g., via sutures 1002) such that the damping member 202 spans the resected portion of the artery A. In some embodiments, the damping member 202 may have a generally cylindrical shape with a constant wall thickness, as shown in FIG. 11. In such embodiments, an inner diameter ID of the damping member 202 may be generally constant along the length of the damping member 202. In operation, the damping devices 1000 and 1100 shown in FIGS. 10 and 11 are highly flexible, elastic members that expand radially outward as the systolic pressure wave passes through the damping devices 1000 and 1100. Since the resected portions of the arterial wall A cannot limit the expansion of the damping devices 1000 and 1100, these devices can expand more than the native arterial wall A to absorb more energy from the blood flow.

### III. SELECTED ADDITIONAL EMBODIMENTS OF DAMPING DEVICES

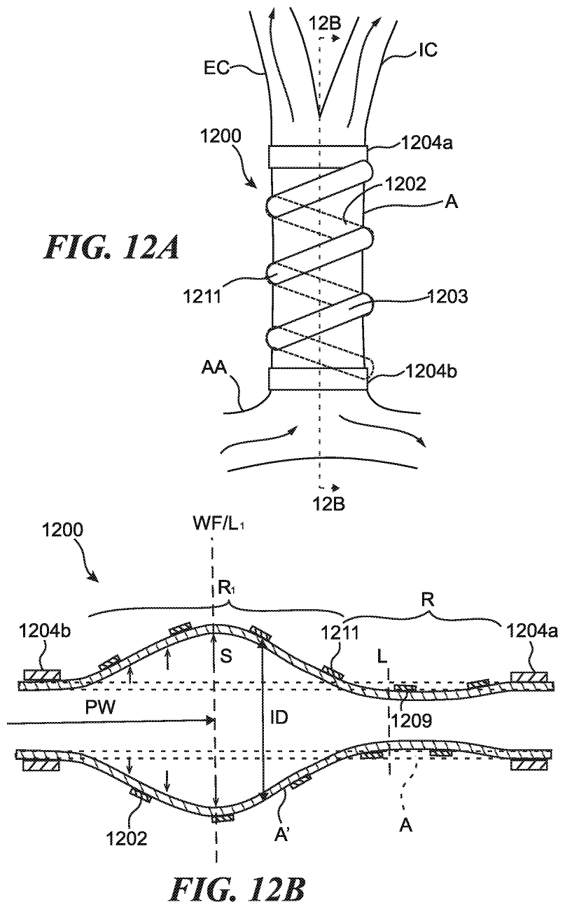

FIGS. 12A-19B illustrate additional embodiments of damping devices configured in accordance with the present technology. For example, FIG. 12A shows a damping device 1200 comprising a damping member 1202 coupled to anchoring members 1204a and 1204b at its proximal and distal end portions. The damping member 1202 comprises a strand 1203 having a pre-set helical configuration such that, in a deployed state, the strand 1203 forms a generally tubular structure defining a lumen extending therethrough. The tubular structure has an inner surface 1209 (FIG. 12B) and an outer surface 1211. The strand 1203 may be formed of any suitable biocompatible material such as one or more elastic polymers that are configured to stretch in response to the radially outward forces exerted by the pulse wave on the helical strand. In some embodiments, the strand 1203 may additionally or alternatively include one or more metals such as stainless steel and/or a superelastic and/or shape memory alloy, such as Nitinol. In a particular embodiment, the damping member 1202 may be fabricated from a recombinant human protein such as tropo-elastin or elastin.

The anchoring members 1204a and 1204b can be generally similar to the anchoring members 104a and 104b described with respect to FIGS. 2A-2C. In some embodiments, the damping device 1200 includes more or fewer than two anchoring members 1204 (one anchoring member, three anchoring members, etc.). In a particular embodiment, the damping device 1200 does not include anchoring members 1204.

In the deployed state, the damping member 1202 is configured to be wrapped along the circumference of an artery that supplies blood to the brain. For example, in the embodiment shown in FIG. 12A, the damping member 1202 is configured to be positioned around the exterior of the artery A such that the inner surface 1209 of the damping member 1202 contacts an outer surface of the artery A (see FIG. 12B). In other embodiments (not shown), the damping member 1202 is configured to be positioned around the lumen of the artery such that the outer surface 1211 of the damping member 1202 contacts an inner surface of the arterial wall.

FIG. 12B is a cross-sectional side view of the damping device 1200 during transmission of a pulse wave PW through the portion of the artery A surrounded by the damping device 1200. In FIG. 12B, the dashed lines A represent the artery during diastole, or when the artery is relaxed. The solid line A′ represents the artery in response to a pulse wave PW traveling through the artery during systole. As shown in FIG. 12B, as the wave front WF (or leading edge of the pulse wave PW) travels through the artery, the wave front dilates the artery A at an axial location L₁ corresponding to the wave front WF. The wave front WF pushes the arterial wall radially outwardly against the coil, thereby radially expanding the portion R₁ of the coil axially aligned with the wave front WF. For example, in those embodiments where the strand 1203 is made of a stretchable material, such as an elastic polymer, the coil stretches along the portion R₁ to expand and accommodate the pulse wave, thereby absorbing some of the energy transmitted with the pulse wave and reducing the stress on the arterial wall. In any of the above embodiments, the portions of the coil distal or proximal the wave-affected region are forced to contract (R₂), thereby causing the artery to narrow relative to its relaxed diameter. This narrowing of the artery creates a temporary impedance to the pulse wave which absorbs some of the energy. Once the pulse wave has passed, the arterial wall returns to its relaxed state.

FIG. 13 illustrates another embodiment of a damping device 1300 in accordance with the present technology. As shown in FIG. 13, the damping device 1300 can include a damping member 1302 defined by an extravascular wrap. The damping member 1302 may be fabricated from a generally rectangular portion of a suitable bio-compatible and elastically deformable material which is configured to be wrapped around the blood vessel. Alternatively, the damping member 1302 may be initially provided having a cylindrical configuration including a longitudinal slit 1304 for receiving the vessel. The damping member 1302 may be fabricated from a synthetic such as an elastic polymer, a shape memory and/or superelastic material such as Nitinol (nickel titanium), a recombinant human protein such as tropo-elastin or elastin, and other suitable materials. As shown in FIG. 13, the damping member 1302 is configured to be secured around an artery (e.g., a carotid artery) between the aortic arch and the junction where the left common carotid artery divides into the internal (IC) and external (EC) carotid arteries. It will be appreciated by those skilled in the art that the damping member 1302 may alternatively or additionally be deployed around the brachiocephalic trunk (not shown) or the right common carotid artery (not shown), or any distal branch of the aforementioned arteries, or any proximal branch of the aforementioned arteries, such as the ascending aorta. Opposing edges of the damping member 1302 can be secured to each other with a coupling device such as stitching/sutures 1310, stapling, or another coupling device such that the external diameter of the artery is reduced. In some embodiments, the coupling device can be made from an clastic material so that it can stretch to accommodate the pulse wave and absorb its energy. The elastically deformable damping member 1302 is adapted to radially expand during the systole stage and radially contract during the diastole stage. The damping member 1302 is secured such that an internal diameter of the elastically deformable material is smaller than an initial, outer diameter of the artery during a systole stage, but not smaller than an outer diameter of the artery during a diastole stage.

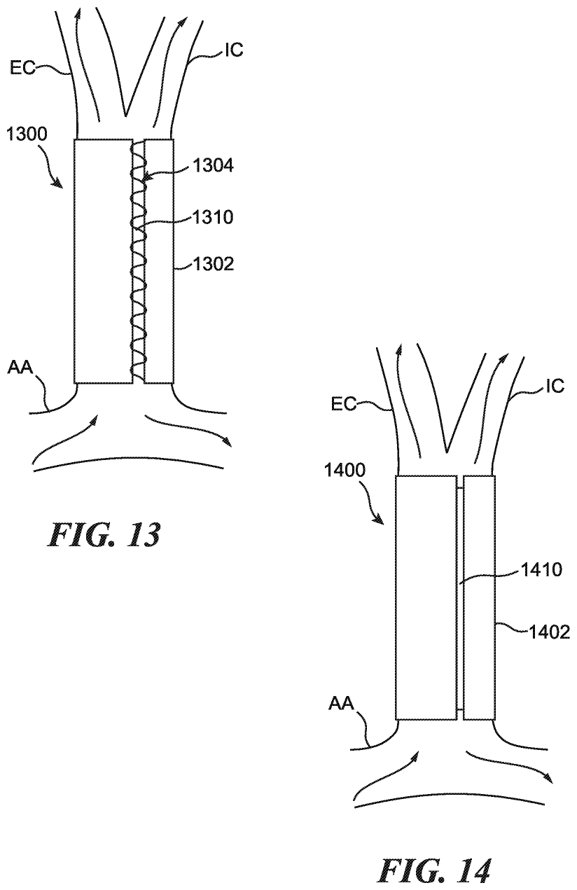

FIG. 14 depicts another embodiment of a damping device 1400 for treating an arterial blood vessel. The device 1400 can be structurally similar to the damping device 1300 shown in FIG. 13, with the exception that the two opposing edges of the elastically deformable damping member 1402 of FIG. 14 are secured to each other using a zip-lock type coupling mechanism 1410.

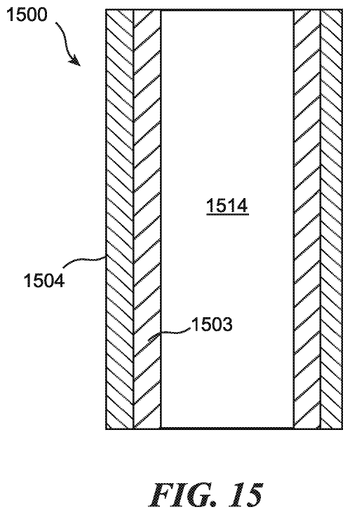

FIG. 15 shows another embodiment of a damping device 1500 configured in accordance with the present technology. The damping device 1500, includes a generally tubular anchoring member 1504 (e.g., a stent, a mesh, a braid, etc.) defining a lumen 1514 therethrough. The anchoring member may be made of a resilient, biocompatible material such as stainless steel, titanium, nitinol, etc. In some embodiments, the anchoring member 1504 is made of a shape memory and/or superelastic material. A radially outer surface of the anchoring member 1504 is configured to be positioned in apposition with an inner surface of an arterial wall. A radially inner surface of the anchoring member 1504 is lined or otherwise coated with an absorptive material 1503 (e.g., a cushioning material), such as an elastically deformable material, which is adapted to absorb shock. The lumen 1514 is configured to receive blood flow therethrough. The lumen 1514 is present when the anchoring member 1504 is radially expanded, but it may not be present in the initial, contracted configuration prior to deployment.

In some embodiments (not shown), the damping device can be a biocompatible gel which is injected around a portion of the left or right carotid artery or the brachiocephalic trunk. The gel increases the external pressure acting on the artery and thus reduces the external diameter of the artery. As blood pressure increases within the artery, the gel elastically deforms, such that the artery radially expands during the systole stage and radially contracts during the diastole stage.

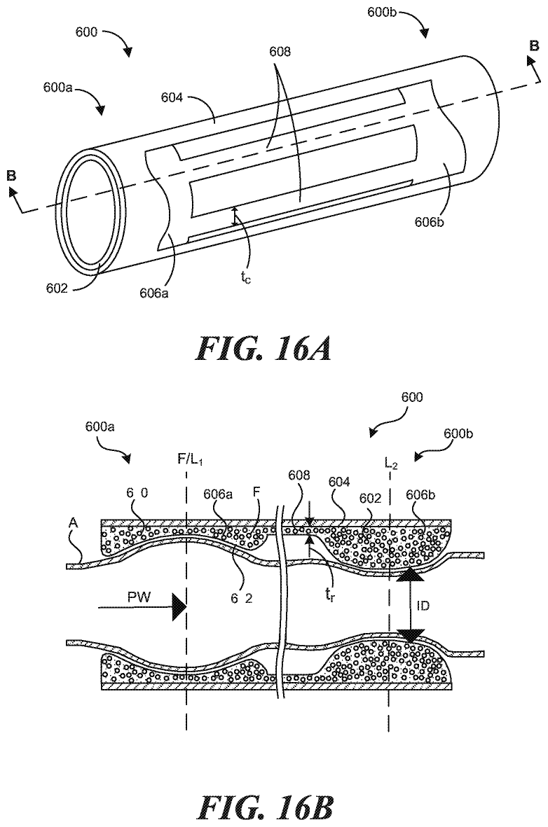

FIG. 16A is a perspective, cut-away view of a damping device 1600 in accordance with the present technology in a deployed, relaxed state. FIG. 16B is a cross-sectional view of the damping device 1600 positioned in an artery A during transmission of a pulse wave PW through the portion of the artery A surrounded by the damping device 1600. Referring to FIGS. 16A and 16B together, the damping device 1600 includes a damping member 1602 and a structural member 1604 coupled to the damping member 1602. In FIG. 16A, a middle portion of the structural member 1604 has been removed to show features of the structure of the damping member 1602. As shown in FIG. 16A, the damping device 1600 can have a generally cylindrical shape in the deployed, relaxed state. The damping device 1600 may be configured to wrap around the circumference of the artery with opposing longitudinal edges (not shown) secured to one another via sutures, staples, adhesive, and/or other suitable coupling devices. Alternatively, the damping device 1600 can have a longitudinal slit for receiving the artery therethrough. In either of the foregoing extravascular embodiments, the damping device 1600 is configured to be positioned around the circumference of the artery A so that the inner surface 1612 (FIG. 16B) is adjacent and/or in contact with the outer surface of the arterial wall. In other embodiments, the damping device 1600 can be configured to be positioned intravascularly (e.g., within the artery lumen) such that an outer surface of the damping device 1600 is adjacent and/or in contact with the inner surface of the arterial wall. In such intravascular embodiments, the inner surface 1612 of the damping member 1602 is adjacent or directly in contact with blood flowing through the artery A.

The structural member 1604 can be a generally cylindrical structure configured to expand from a low-profile state to a deployed state. The structural member 1604 is configured to provide structural support to secure the damping device 1600 to a selected region of the artery. In some embodiments, the structural member 1604 can be a stent formed from a laser cut metal, such as a superelastic and/or shape memory material (e.g., Nitinol) or stainless steel. All or a portion of the structural member 1604 can include a radiopaque coating to improve visualization of the device 1600 during delivery, and/or the structural member 1604 may include one or more radiopaque markers. In other embodiments, the structural member 1604 may comprise a mesh or woven (e.g., a braid) construction in addition to or in place of a laser cut stent. For example, the structural member 1604 can include a tube or braided mesh formed from a plurality of flexible wires or filaments arranged in a diamond pattern or other configuration. In some embodiments, all or a portion of the structural member 1604 can be covered by a graft material (such as Dacron) to promote sealing with the vessel wall. Additionally, all or a portion of the structural member 1604 can include one or more biomaterials.

In the embodiment shown in FIGS. 16A and 16B, the structural member 1604 is positioned radially outwardly of the damping member 1602 and extends along the entire length of the damping member 1602 (though a middle portion of the structural member 1604 is cut-away in FIG. 16A for illustrative purposes only). In other embodiments, the structural member 1604 and the damping member 1602 may have other suitable configurations. For example, the damping device 1600 can include more than one structural member 1604 (e.g., two structural members, three structural members, etc.). Additionally, in some embodiments the structural member(s) 1604 may extend along only a portion of the damping member 1602 such that a portion of the length of the damping member 1602 is not surrounded and/or axially aligned with any portion of the structural member 1604. Also, in some embodiments, all or a portion of the damping member 1602 may be positioned radially outwardly of all or a portion of the structural member 1604.

In the embodiment shown in FIGS. 16A and 16B, the damping member 1602 includes a proximal damping element 1606a and a distal damping element 1606b. The damping member 1602 may further include optional channels 1608 extending between the proximal and distal damping elements 1606a, 1606b. The channels 1608, for example, can extend in a longitudinal direction along the damping device 1600 and fluidly couple the proximal damping element 1606a to the distal damping element 1606b. The damping member 1602 may further include an abating substance 1610 configured to deform in response to fluid stress (such as blood flow), thereby absorbing at least a portion of the stress. For example, as best shown in FIG. 16B, in one embodiment the abating substance 1610 includes a plurality of fluid particles F (only one fluid particle labeled) contained in the proximal damping element 1606a, distal damping element 1606b, and channel(s) 1608. As used herein, the term “fluid” refers to liquids and/or gases, and “fluid particles” refers to liquid particles and/or gas particles. In some embodiments, the damping member 1602 is a gel, and the plurality of fluid particles F are dispersed within a network of solid particles. In other embodiments, the damping member 1602 may include only fluid particles F (e.g., only gas particles, only liquid particles, or only gas and liquid particles) contained within a flexible and/or elastic membrane that defines the proximal damping member 1606a, the distal damping member 1606b, and the channel(s) 1608. The viscosity and/or composition of the abating substance 1610 may be the same or may vary along the length and/or circumference of the damping member 1602.

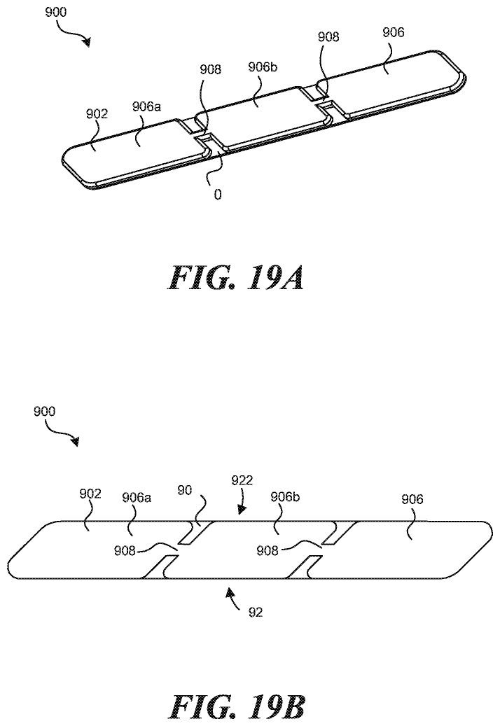

In the embodiment shown in FIGS. 16A and 16B, the channels 1608 have a resting radial thickness tᵣ and circumferential thickness t_{c} (FIG. 16A) that is less than the resting radial thickness tᵣ and circumferential thickness t_{c}, respectively, of the proximal and distal damping elements 1606a, 1606b. As best shown in FIG. 16A, in some embodiments the proximal and distal damping elements 1606a and 1606b may extend around the full circumference of the damping device 1600 and the channels 1608 may extend around only a portion of the circumference of the damping device 1600. In other embodiments, the channels 1608 can have a resting radial thickness tᵣ that is generally the same as that of the proximal and distal damping elements 1606a, 1606b (see damping elements 1906a-c and channels 1908 in FIGS. 19A and 19B) and/or a resting circumferential thickness t_{c} that is generally the same as that of the proximal and distal damping elements 1606a, 1606b.

Referring to FIG. 16B, when a pulse wave PW traveling through the artery A applies a stress at a first axial location L₁ along the length of the damping member 1602 (e.g., at wave front WF), at least a portion of the fluid particles move away from the first axial location L₁ to a second axial location L₂ along the length of the damping member 1602. As such, at least a portion of the fluid particles are redistributed along the length of the damping member 1602 such that the inner diameter ID of the damping member 1602 increases at the first axial location L₁ while the inner diameter ID decreases at another axial location (e.g., L₂). For example, as the wave front WF passes through the proximal portion 1600a of the device 1600, the portion of the artery A aligned with the wave front WF dilates, thereby applying a stress to the proximal damping element 1606a and forcing at least some of the fluid particles in the proximal damping element 1606a to move distally within the damping member 1602. At least some of the displaced fluid particles are forced through the channel(s) 1608 and into the distal damping element 1606b, thereby increasing the volume of the distal damping element 1606b and decreasing the inner diameter ID of the damping device 1600 at the distal portion 1600b. The decreased inner diameter ID of the damping device 1600 provides an impedance to the blood flow that absorbs at least a portion of the energy in the pulse wave when the blood flow reaches the distal damping member 1606b. As the wave front WF then passes through the distal portion 1600b of the device 1600, the portion of the artery A aligned with the wave front WF dilates, thereby applying a stress to the distal damping element 1606b and forcing at least some of the fluid particles currently in the distal damping element 1606b to move proximally within the damping member 1602. At least some of the displaced fluid particles are forced through the channel(s) 1608 and into the proximal damping element 1606a, thereby increasing the volume of the proximal damping element 1606a and decreasing the inner diameter ID of the device 1600 at the proximal portion 1600a. Movement of the fluid particles and/or deformation of the damping member 1602 in response to the pulse wave absorbs at least a portion of the energy carried by the pulse wave, thereby reducing the stress on the arterial wall distal to the device.

When the damping member 1602 deforms in response to the pulse wave, the shape of the structural member 1604 may remain generally unchanged, thereby providing the support to facilitate redistribution of the fluid particles within and along the damping member 1602. In other embodiments, the structural member 1604 may also deform in response to the local fluid stress.

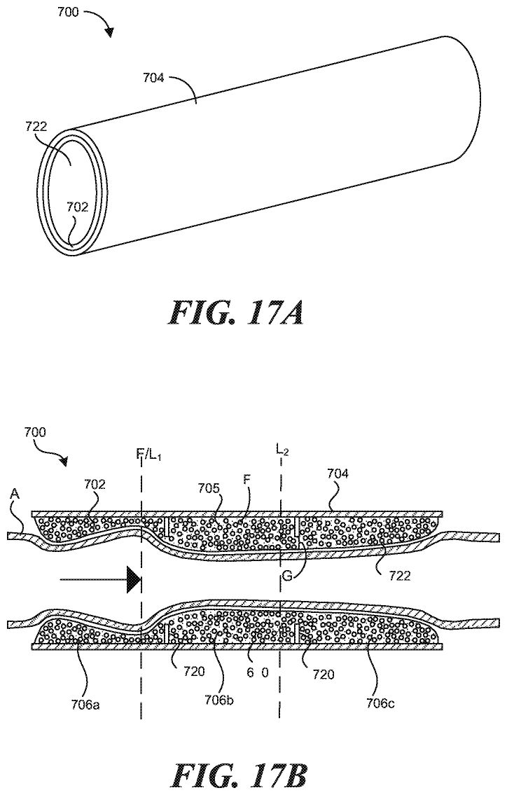

FIG. 17A is a perspective view of another embodiment of a damping device 1700 in accordance with the present technology. FIG. 17B is a cross-sectional view of the damping device 1700 positioned in an artery A during transmission of a pulse wave PW through the portion of the artery A surrounded by the damping device 1700. The damping device 1700 can include a structural member 1704 and a damping member 1702. The structural member 1704 can be generally similar to the structural member 1604 shown in FIGS. 16A and 16B. The damping member 1702 is defined by a single chamber 1705 including an abating substance 1610 and a plurality of baffles 1720 that separate the chamber 1705 into three fluidically-coupled compartments 1706a, 1706b, and 1706c. The baffles 1720 extend only a portion of the radial thickness of the damping member 1702, thereby leaving a gap G between the end of the baffles 1720 and an inner wall 1722 of the damping member 1702. In other embodiments, the damping device 1700 can include more or fewer compartments (e.g., a single, tubular compartment (no baffles), two compartments, four compartments, etc.). Moreover, the baffles 1720 may extend around all or a portion of the circumference of the damping member 1702.

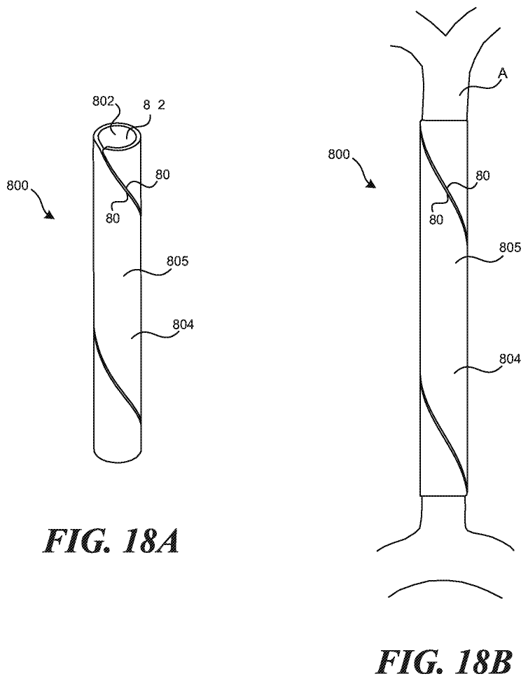

FIG. 18A is a perspective view of another embodiment of a damping device 1800 in accordance with the present technology, and FIG. 18B is a front view of the damping device 1800, shown in a deployed state positioned around an artery A. Referring to FIGS. 18A-18B together, the damping device 1800, in a deployed, relaxed state, includes a generally tubular sidewall 1805 that defines a lumen. The damping device 1800 can be formed of a generally parallelogram-shaped element that is wrapped around a mandrel in a helical configuration and heat set. In other embodiments, the damping device 1800 can have other suitable shapes and configurations in the unfurled, non-deployed state. As shown in FIG. 18B, in the deployed state, the damping device 1800 is configured to be wrapped helically along or around the circumference of an artery supplying blood to the brain. Opposing longitudinal edges 1807 of the damping device 1800 come together in the deployed state to form a helical path along the longitudinal axis of the artery A. The damping device 1800 can include any of the coupling devices described with respect to FIGS. 13-15 to secure all or a portion of the opposing longitudinal edges to one another.

As best shown in FIG. 18A, the sidewall 1805 of the damping device 1800 includes a structural member 1804 and a damping member 1802. The structural member 1804 can be generally similar to the structural member 1604 shown in FIGS. 16A and 16B, except the structural member 1804 of FIGS. 18A and 18B has a helical configuration in the deployed state. The damping member 1802 can be generally similar to any of the damping members described herein, especially those described with respect to FIGS. 13-17B and 19A and 19B. In the embodiment shown in FIGS. 18A and 18B, the damping member 1802 is positioned radially inwardly of the structural member 1804 when the damping device 1800 is in the deployed state. In other embodiments, the damping member 1802 may be positioned radially outwardly of the structural member 1804 when the damping device 1800 is in the deployed state.

The damping device 1800 may be configured to wrap around the circumference of the artery A so that the inner surface 1812 (FIG. 18A) is adjacent and/or in contact with the outer surface of the arterial wall. In other embodiments, the damping device 1800 can be configured to be positioned intravascularly (e.g., within the artery lumen) such that an outer surface of the damping device 1800 is adjacent and/or in contact with the inner surface of the arterial wall. In such intravascular embodiments, the inner surface 1812 of the damping member 1802 is adjacent or directly in contact with blood flowing through the artery A.

FIGS. 19A and 19B are perspective and top views, respectively, of a damping device 1900 that can define one embodiment of the damping device 1800 shown in FIGS. 18A and 18B. In FIGS. 19A and 19B, the damping device 1900 is shown in an unfurled, non-deployed state. The damping device 1900 includes a damping member 1902 having a plurality of chambers 1906a, 1906b, 1906c spaced apart along a longitudinal dimension of the damping device 1900 in the unfurled state. The chambers 1906a, 1906b, 1906c may be fluidly coupled by channels 1908 extending between adjacent chambers. The damping device 1900 can thus operate in a manner similar to the damping device 1600 where an abating substance (not shown in FIGS. 19A and 19B) in the chambers 1906a-c moves through the channels 1908 to inflate/deflate individual chambers in response to a pressure wave traveling through the blood vessel. The displacement of the abating substance within the chambers 1906a-c attenuates the energy of the pulse wave to reduce the impact of the pulse wave distally of the damping device 1900.

### IV. SELECTED THERAPEUTIC AGENTS FOR TREATING NEUROLOGICAL CONDITIONS

In addition to providing the implantable damping device, the present technology includes providing therapeutic agents for treating neurological disorders. One of ordinary skill in the art will understand that the therapeutic agents discussed herein are illustrative of the type of therapeutic agents in the present technology, and that the present technology is not limited to the therapeutic agents explicitly discussed herein. For example, therapeutic agents not explicitly described herein but that are within the classes of therapeutic agents provided herein and/or treat the neurological conditions discussed herein are included in the present technology.

Therapeutic agents for treating neurological conditions, such as neurocognitive and/or neurodegenerative disorders, include therapeutic agents approved for use in human subjects by the Food and Drug Administration of the United States of America (“FDA”), therapeutic agents currently in clinical trials to investigate their use in human subjects such as clinical trials governed by the FDA or other similar organizations in other countries, pre-clinical therapeutic agents, and any other therapeutic agent for treating a neurological condition, or intended to treat a neurological condition such as investigative therapeutic agents, therapeutic agents that are undergoing development or otherwise being considered for development, and therapeutic agents that have been identified as potentially useful for treating or intending to treat the neurological condition. Examples of neurological conditions, such neurocognitive, neurodegenerative, or other neurological disorders include, but are not limited to, Alzheimer's disease, mild Alzheimer's disease, prodromal Alzheimer's disease, mild cognitive impairment, cerebral amyloid angiopathy, frontotemporal dementia, vascular dementia, age-related dementia, amyloidosis, Lewy body disease, Parkinson's disease, Huntington's disease, multiple sclerosis, amyotrophic lateral sclerosis, Friedreich's ataxia, and traumatic brain injury. In some embodiments, these therapeutic agents represent more than one class of therapeutic agents, more than one mechanism of action, more than one therapeutic target, and more than one therapeutic purposes.

The therapeutic agents discussed herein have different therapeutic purposes, such as disease modifying therapeutic agents, symptomatic cognitive enhancers, and/or symptomatic agents addressing neuropsychiatric and behavioral changes. Disease modifying therapeutic agents, for example, alter the pathophysiology of the neurological condition. Symptomatic therapeutic agents, for example, mitigate and/or alleviate symptoms associated with the neurological condition. In some embodiments, a therapeutic agent is a disease modifying therapy and a symptomatic therapy. In some embodiments, a therapeutic agent may include more than one therapeutic agent.

In some embodiments, therapeutic agents of the present technology are members of general classes of therapeutic agents which include, but are not limited to, EDG Receptor Family modulators, MMP inhibitors, and senolytic agents.

In some embodiments, therapeutic agents of the present technology have different mechanisms of action. In some embodiments, a therapeutic agent is selected for administration to a subject in need thereof based on its mechanism of action. For example, some therapeutic agents for treating neurological conditions such as Alzheimer's disease prevent abnormal cleavage of amyloid precursor protein in a subject's brain. In some embodiments, therapeutic agents prevent expression and/or accumulation of β-amyloid protein (Aβ) in the subject's brain. For example, some therapeutic agents for treating neurological conditions such as Alzheimer's disease reduce systemic inflammation, blood brain barrier inflammation, systemic inflammation, blood brain barrier inflammation, or neuroinflammation in the subject. In some embodiments, therapeutic agents for treating neurological conditions such as Alzheimer's disease have an endothelial protective effect. In some embodiments, therapeutic agents treat, prevent, or delay Alzheimer's disease, other neurological conditions, or cognitive decline by decreasing inflammation (e.g., blood brain barrier inflammation, neuroinflammation, systemic inflammation), decreasing reactive oxygen species (e.g., oxidative stress), decreasing ischemia, and/or decreasing amyloid beta. For example, inflammation can be decreased by directly or indirectly reducing a level of one or more cytokines, chemokines, and/or inflammatory markers in the subject (e.g., circulating or in the subject's cerebral spinal fluid (CSF)), including VCAM-1, ICAM-1, TNFα, TGF-β, IL-6, IL-8, IL-1β, IL-12, and NF-κB, and/or administering a peptide-based therapeutic to the subject, such as an IL-1Rα derivative (e.g., Anakinra®). Reactive oxygen species can be increased in the subject relative to before the subject has the condition or compared to a subject who does not have the condition. In other embodiments, therapeutic agents treat, prevent, or delay Alzheimer's disease, other neurological conditions, or cognitive decline by inhibiting MMPs, eliminating senescent cells, improving tight junctions, or inducing transcriptional activation of TIMP.

Any of the therapeutic agents described herein, as well as other therapeutic agents which are members of the general classes of therapeutic agents described herein, are administered to the subject in need thereof at a therapeutically effective dose before, during, or after positioning the implantable damping device within a subject, such as but not limited to a subject having elevated forward compression wave intensity (FCWI) or elevated pulse pressure. Non-limiting elevated pulse pressure values include top-quartile carotid FCWI (mmHg m/s³) of at least about 10,000, a systolic blood pressure (mmHg) of at least about 125 mmHg, a pulse pressure of at least about 50 mmHg, a diastolic blood pressure of at most about 70 mmHg, and/or a mean arterial pressure of at least about 88 mmHg. In some embodiments, a carotid FCWI range (mmHg m/s³) of at least about 10,000, a systolic blood pressure (mmHg) of at least about 135 mmHg, a pulse pressure of at least about 55 mmHg, a diastolic blood pressure of at most about 75 mmHg, and/or a mean arterial pressure of at least about 95 mmHg. Without intending to be bound by any particular dose or administration regimen, a therapeutically effective dose is an amount of the therapeutic agent that, when administered to the subject in need thereof, treats or at least partially treats, reduces the effects of, or at least partially reduces the effects of, the subject's condition (e.g., neurodegenerative condition, cognitive decline). The therapeutically effective dose for each therapeutic agent is selected based upon a variety of factors, including but not limited to, one or more characteristics of the therapeutic agent (e.g., bioactivity, pharmacokinetics, pharmacodynamics, and bioavailability), the physiological condition of the subject (e.g., age, sex, disease type and stage, general physical condition, responsiveness to a given dosage, and type of medication), and the route of administration.

### A. EDG Receptor Family Modulators

Lysophosphatidic acid (LPA) and sphingosine-1-phosphate (S1P) are lysophospholipids (LPLs) that can have similar effects on cellular response by acting on a common family of receptors. The Endothelial Cell Differentiation Gene (EDG) Receptors are a family of G-Protein Coupled Receptor (GPCR) that function as receptors for both S1P and LPA. EDG receptors, identified in Table 1 below, are classed into two sub-families and named in accordance with the ligand to which they bind (S1P or LPA).

TABLE 1
EDG Receptor Family

| EDG Receptor | Name | Ligand |
|---|---|---|
| EDG1 | Sphingosine 1-phosphate receptor 1 (S1PR1) | S1P |
| EDG2 | Lysophosphatidic acid receptor 1 (LPAR1) | LPA |
| EDG3 | Sphingosine 1-phosphate receptor 3 (S1PR3) | S1P |
| EDG4 | Lysophosphatidic acid receptor 2 (LPAR2) | LPA |
| EDG5 | Sphingosine 1-phosphate receptor 2 (S1PR2) | S1P |
| EDG6 | Sphingosine 1-phosphate receptor 4 (S1PR4) | S1P |
| EDG7 | Lysophosphatidic acid receptor 3 (LPAR3) | LPA |
| EDG8 | Sphingosine 1-phosphate receptor 5 (S1PR5) | S1P |

Activation of the EDG receptors by S1P or LPA has multiple effects on cells, including proliferation, cell survival, migration, differentiation and cytoskeletal organization, and cytokine secretion. In addition to these effects, these S1P and LPA have been implicated in many pathophysiological processes, including inflammatory vascular disease, autoimmune disease, fibrotic disease, cancer, inflammation, and bone disease. Thus, EDG receptor family modulators (e.g., S1P receptor agonists, LPA receptor agonists) may be used in accordance with the embodiments described herein.

S1P has been shown to enhance endothelial barrier function, stimulate endothelial NO release through Akt-mediated phosphorylation of eNOS, and reconstitute high density lipoproteins. S1P also has anti-inflammatory properties and exerts protective effect against endotoxin-induced lung injury. Moreover, S1P exhibits a potent effect on the differentiation of adipose-derived stem cells into endothelial-like cells and upregulation of eNOS in these cells. These properties of S1P may contribute to its endothelial protective effect. Thus, in some embodiments, the EDG receptor family modulator is an S1P receptor agonist.

In some embodiments, the S1P receptor agonist is fingolimod (also known as FTY720, or Gilenya®), siponimod (Mayzent®), ozanimod, or ponesimod. These S1P receptor agonists may be beneficial to blood brain barrier integrity through activation of S1P1 and/or S1P5. In certain embodiments, the S1P receptor agonist is fingolimod (also known as FTY720, or Gilenya®). FTY720, after phosphorylation to FTY720-P, is an orally active S1P mimetic. Fingolimod was developed for therapy in the field of autoimmune diseases and organ transplantation, and is a clinically approved immunomodulating therapy for multiple sclerosis. Fingolimod shows potent anti-inflammatory effects and S1P receptor activation may lead to inhibition of the progression of inflammatory vascular diseases like atherosclerotic lesions. Fingolimod has also been shown to promote angiogenesis, attenuate ischemic brain damage, repair brain injuries, and may have the ability to reverse blood-brain-barrier damage (or to preserve BBB integrity). Fingolimod has also been shown to attenuate infection-induced enhancement of Aβ Accumulation. In other embodiments, the S1P receptor agonist is siponimod. Similar to fingolimod, siponimod has been indicated to strengthen BBB properties by modulating S1P1 and S1P5.

EDG receptor family modulators can be administered at any therapeutically effective dose that is effective to treat the subject in need thereof. In certain embodiments, the effective dose corresponds to an FDA-approved dose or any off-label use within the standard of care known to those skilled in the art. For example, in certain embodiments where fingolimod and/or siponimod is used, the fingolimod and/or siponimod may be administered as recommended at a dose of 0.25 mg, 0.5 mg, 0.75 mg, 1.0 mg, 1.25 mg, or 2 mg. In other embodiments where ponesimod is used, the ponesimod may be administered as recommended at a dose of 1 mg, 2 mg, 3 mg, 4 mg, 5 mg, 6 mg, 7 mg, 8 mg, 9 mg, 10 mg, or 20 mg. In other embodiments where ozanimod is used, the ozanimod may be administered as recommended at a dose of 0.23 mg, 0.46 mg, or 0.92 mg.

In other embodiments, the effective dose corresponds to a dose that is lower or higher than the FDA-approved dose. For example, in some embodiments, a dose of an EDG receptor family modulator ranges from about 1 to about 500 μg/kg of body weight. For example, dosages are between about 1 μg/kg and about 10 μg/kg, between about 0.1 μg/kg and about 50 μg/kg, between about 1 μg/kg and about 100 μg/kg, between about 1 μg/kg and about 150 μg/kg, or between about 1 μg/kg and about 200 mg/kg, about 1 μg/kg and about 250 μg/kg, about 1 μg/kg and about 300 μg/kg, about 1 μg/kg and about 350 μg/kg, about 1 μg/kg and about 400 μg/kg, about 1 μg/kg and about 450 μg/kg, about 1 μg/kg and about 500 μg/kg, about 1 μg/kg and about 750 μg/kg, or about 1 μg/kg and about 1 mg/kg, or about 1 μg/kg and about 2 mg/kg. For example, dosages also include one or more doses of about 1 μg/kg, about 2.5 μg/kg, about 5 μg/kg, about 10 μg/kg, about 20 μg/kg, about 30 μg/kg, about 40 μg/kg, about 50 μg/kg, about 100 μg/kg, about 150 μg/kg, about 200 μg/kg, about 250 μg/kg, about 300 μg/kg, about 350 μg/kg, about 400 μg/kg, about 450 μg/kg, about 500 μg/kg, about 750 μg/kg, about 1 mg/kg, or about 2 mg/kg. (or any combination thereof). In some embodiments, the EDG receptor family modulators are administered at a flat dose of less than about 0.001 mg, about 0.001 mg, about 0.01 mg, about 0.1 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, or higher than about 20 mg.

In some embodiments, the EDG receptor family modulator is administered once daily, twice daily, or more than twice daily. In other embodiments, the EDG receptor family modulator is administered less often, e.g., once a week, twice a week, three times a week, four times a week, five times a week, or six times a week. In certain embodiments, the EDG receptor family modulator may be titrated, wherein the EDG receptor family modulator is administered starting at a low dose, and is then raised incrementally until the desired effective dose is reached. In some embodiments, the EDG receptor family modulator is administered chronically. In some embodiments, dosages of EDG receptor family modulators are administered in one or more separate administrations or by continuous infusion.

### B. Matrix Metalloproteinase Inhibitors

Matrix metalloproteinases (MMPs) encompass 23 secreted or cell surface proteases that act together and with other protease classes to turn over the extracellular matrix, cleave cell surface proteins and alter the function of many secreted bioactive molecules. In the vasculature, MMPs influence the migration proliferation and apoptosis of vascular smooth muscle, endothelial cells and inflammatory cells, thereby affecting intima formation, atherosclerosis and aneurysms. MMP inhibitors have thus been investigated for use in treating vascular diseases. For example, naturally occurring tissue inhibitors of MMPs (TIMPs), pleiotropic mediators such as tetracyclines, chemically-synthesized small molecular weight MMP inhibitors (MMPs) and inhibitory antibodies have all been shown to have effects in animal models of vascular disease. MMP inhibitors may therefore be used in accordance with the embodiments described herein. In some embodiments, the MMP inhibitor is ilomastat, marimastat, prinomastat, batimastat, cipemastat, andecaliximab, or doxycycline. In certain embodiments, the MMP inhibitor is doxycycline.

In addition to such agents that directly inhibit MMPs, other agents may act to indirectly inhibit MMP-mediated processes. For example, glucocorticoids have been investigated as a therapeutic intervention for protecting tight junctions and avoiding BBB disruption in a number of cells and animal models. Glucocorticoids, such as dexamethasone, hydrocortisone, and corticosterone, induce improved tight junctions through an increased level of tight junction proteins in vascular endothelial cells. This improvement in the tight junctions by glucocorticoids is also associated with a rearrangement of the cytoskeleton. Moreover, dexamethasone in particular has been shown to inhibit the cytokine-induced upregulation of MMP-9. Dexamethasone also induces transcriptional activation of TIMP-1 (tissue inhibitor of metalloproteinase-1) and TIMP-3, which may block MMP-9-mediated degradation of tight junction proteins. Additionally, dexamethasone suppresses JMJD3 gene activation via a putative negative glucocorticoid response element and maintains integrity of tight junctions in brain microvascular endothelial cells. Further, dexamethasone administration following BBB disruption has been found to expedite the restoration of BBB integrity and to prevent a subsequent elevation in the production of inflammatory markers. Thus, glucocorticoids like dexamethasone may be used in accordance with the embodiments described herein to serve as both an anti-inflammatory agent as well as an indirect MMP inhibitor.

MMP inhibitors, whether direct or indirect can be administered at any therapeutically effective dose that is effective to treat the subject in need thereof. In certain embodiments, the effective dose corresponds to an FDA-approved dose or any off-label use within the standard of care known to those skilled in the art. For example, in some embodiments, where doxycycline is used, the doxycycline may be administered as recommended at a dose of 20 mg, 25 mg, 40 mg, 50 mg, 60 mg, 75 mg. 80 mg, 100 mg, 120 mg, 150 mg, 200 mg, 240 mg, or 300 mg; a daily dosage of 100 mg/day, 100-200 mg/day, or 200 mg/day. In other embodiments where dexamethasone is used, the dexamethasone may be administered as recommended at a dose of 0.5 mg, 0.75 mg, 1 mg, 1.5 mg, 2 mg, 3 mg, 4 mg, 6 mg, 7 mg, 8 mg, 10 mg, 20 mg; a dose within a range of 4-8 mg, 8-12 mg, 4-24 mg, 10-100 mg; a daily dosage of 0.2-6 mg/day, 0.75-9 mg/day, 3 mg/day, 30 mg/day; or a dose by weight of 1-6 mg/kg or 3 mg/kg.

In other embodiments, the effective dose corresponds to a dose that is lower or higher than the FDA-approved dose. For example, in some embodiments, a dose of an MMP inhibitor ranges from about 0.1 to about 10 mg/kg of body weight. For example, dosages are between about 0.1 mg/kg and about 1 mg/kg, between about 0.1 mg/kg and about 2 mg/kg, between about 0.1 mg/kg and about 3 mg/kg, between about 0.1 mg/kg and about 4 mg/kg, between about 0.1 mg/kg and about 5 mg/kg, between about 0.1 mg/kg and about 6 mg/kg, between about 0.1 mg/kg and about 7 mg/kg, between about 0.1 mg/kg and about 8 mg/kg, between about 0.1 mg/kg and about 9 mg/kg, or between about 0.1 mg/kg and about 10 mg/kg. For example, dosages also include one or more doses of about 0.1 mg/kg, about 0.2 mg/kg, about 0.25 mg/kg, about 0.3 mg/kg, about 0.4 mg/kg, about 0.5 mg/kg, about 0.75 mg/kg, about 1.0 mg/kg, about 1.5 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10 mg/kg or more than about 10 mg/kg (or any combination thereof). In some embodiments, the MMP inhibitor is administered at a flat dose of about 0.1 mg, about 1.5 mg, about 0.2 mg, about 2.5 mg, about 0.3 mg, about 3.5 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1.0 mg, about 1.5 mg, about 2.0 mg, about 2.5 mg, about 3.0 mg, about 3.5 mg, about 4.0 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 400 mg, about 500 mg, about 1000 mg, or higher.

In some embodiments, the MMP inhibitor is administered once daily, twice daily, or more than twice daily. In other embodiments, the EDG receptor family modulator is administered less often, e.g., once a week, twice a week, three times a week, four times a week, five times a week, or six times a week. In certain embodiments, the EDG receptor family modulator may be titrated, wherein the EDG receptor family modulator is administered starting at a low dose, and is then raised incrementally until the desired effective dose is reached. In some embodiments, the MMP inhibitor is administered chronically. In some embodiments, dosages of the MMP inhibitor are administered in one or more separate administrations or by continuous infusion.

### C. Senolytic Agents

Senolytic agents are molecules that selectively eliminate senescent cells and have typically been investigated as candidates for treating age-related diseases. Non-limiting examples of senolytic agents that can be used in accordance with the embodiments described herein include, but are not limited to, FOXO4-related peptides, bcl-2 inhibitors, Src tyrosine kinase inhibitors, USP7 (Ubiquitin-specific processing protease 7) inhibitors, dasatinib, quercetin, Fisetin, Navitoclax, Piperlongumine, Azithromycin, Roxithromycin, SSK1 (senescence-specific killing compound 1), Crispr/Cas9 used to knockout BIRC5 or other gene sequences (e.g., cancer or damage marker gene sequences), and kidney-type glutaminase 1 (GLS1) inhibitors.

In some embodiments, a combination of senolytic agents can be used in accordance with the methods and treatments discussed herein. In some embodiments, the combination of senolytic agents is a combination of dasatinib and quercetin (D+Q). D+Q has been found to alleviate age-related brain inflammation, Aβ-associated oligodendrocyte progenitor cell senescence, and cognitive deficits in an Alzheimer's disease model.

Senolytic agents (alone or in combination) can be administered at any therapeutically effective dose that is effective to treat the subject in need thereof. In certain embodiments, the effective dose corresponds to an FDA-approved dose or any off-label use within the standard of care known to those skilled in the art. For example, in some embodiments, where dasatinib is used, the dasatinib may be administered as recommended at a dose of 20 mg, 50 mg, 70 mg, 80 mg, 100 mg, 140 mg, 180 mg. In other embodiments, where quercetin is used, the quercetin may be administered as recommended at a dose of 400 mg, 500 mg, 1000 mg, or between 400-1400 mg. In certain embodiments where a combination D+Q is used, the D+Q may be administered at a dose of 100 mg dasatinib in combination with 1000 mg of quercetin, or any combination of recommended doses discussed above.

In other embodiments, doses of a senolytic agent (alone or in combination) ranges from about 0.0001 to about 500 mg/kg of body weight. For example, dosages are between about 0.1 mg/kg and about 500 mg/kg, between about 0.1 mg/kg and about 250 mg/kg, between about 0.1 mg/kg and about 100 mg/kg, between about 0.1 mg/kg and about 50 mg/kg, or between about 0.1 mg/kg and about 25 mg/kg. For example, dosages also include one or more doses of about 0.1 mg/kg, about 0.2 mg/kg, about 0.3 mg/kg, about 0.4 mg/kg, about 0.5 mg/kg, about 1.0 mg/kg, about 1.5 mg/kg, about 2.0 mg/kg, about 3.0 mg/kg, about 4.0 mg/kg, about 5.0 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90/mg/kg, about 100 mg/kg, about 200 mg/kg, about 300 mg/kg, about 400 mg/kg, or about 500 mg/kg (or any combination thereof). In some embodiments, a senolytic agent (alone or in combination) is administered at a flat dose of about 1 mg, about 5 mg, about 10 mg, about 25 mg, about 50 mg, about 100 mg, about 200 mg, about 300 mg, about 500 mg, about 1000 mg, or higher. For example, the senolytic agent (alone or in combination) is administered in one to fifty doses (e.g., the therapy may be delivered in a single dose, in two doses, in three doses, in four doses, in five doses, etc.). In some embodiments, the total dose administered is in the range of about 25 mg to about 5000 mg or higher, of about 50 mg to about 2500 mg, of about 50 mg to about 2000 mg, about 50 mg to about 1500 mg, about 50 mg to about 1000 mg, about 50 mg to about 500 mg, about 50 mg to about 100 mg, or any other range having a therapeutic effect on the subject's condition. For example, the total dose administered can be about 25 mg, about 50 mg, about 100 mg, about 200 mg, about 300 mg, about 400 mg, about 500 mg, about 600 mg, about 700 mg, about 1000 mg, about 1200 mg, about 1500 mg, about 1800 mg, about 2000 mg, about 2500 mg, about 3000 mg, about 4000 mg, about 5000 mg, or higher. In some embodiments, the senolytic agent (alone or in combination) is administered chronically. In some embodiments, dosages of senolytic agent (alone or in combination) are administered in one or more separate administrations or by continuous infusion.

One skilled in the art will understand that the foregoing therapies and accompanying description is for illustrative purposes and does not limit the therapies that may be provided in certain embodiments of the present technology. Accordingly, any therapy useful in or designed to treat a neurological condition, such as a neurodegenerative condition, may be present in certain embodiments of the present technology.

### V. SELECTED METHODS OF TREATING NEUROLOGICAL CONDITIONS WITH A COMBINATION OF AN IMPLANTABLE DAMPING DEVICE AND A THERAPEUTIC AGENT

Reducing a subject's pulse pressure with the implantable damping devices has subsequent downstream impacts on other factors that contribute to onset, duration, and/or progression of the subject's condition (e.g., neurological condition). Combining use of devices of the present disclosure to reduce the subject's pulse pressure with therapeutic agents that, when administered to the subject, enhance an effect of reduced pulse pressure on certain outcomes results in a greater reduction and prevention of onset, duration, and/or progression of the subject's neurological condition. Reducing these outcomes results in reduced inflammation (e.g., neuroinflammation, systemic inflammation, blood brain barrier inflammation), decreasing reactive oxygen species (e.g., oxidative stress), reduced ischemia, restoring blood brain barrier integrity, and other benefits. This occurs in subjects suffering from conditions such as progressive cognitive dysfunction and dementia. Without intending to be limiting, subjects having a FCWI value in the top quartile are thought to benefit from a reduction in the FCWI, systolic acceleration time, systolic pressure, and/or pulse pressure value achieved with the implantable damping devices of the present disclosure.

Several biological pathways, for example such as those described herein, may contribute to a neurological condition (e.g., dementia). Without intending to be bound by any particular theory, it is thought that interfering (e.g., altering, effecting, impairing, inhibiting, reducing, or otherwise changing the function of) two or more biological pathways is more effective for treating, preventing, or otherwise reducing the subject's neurological condition, and/or symptoms thereof, rather than interfering with a single biological pathway. In this way, the effects of combining the implantable damping device and at least one therapeutic agent of the present technology may be complementary, additive or even synergistic when compared to an effect of the implantable damping device and the therapeutic agent alone. Accordingly, combining the implantable damping devices with one or more therapeutic agents that affect these other factors further treats and/or slows one or more effects of the condition.

As described above, combinatorial therapies of the present technology include an implantable damping device and a therapeutic agent for treating and/or preventing the progression of the condition. Some embodiments of the present technology, for example, are directed to combinatorial therapies including the implantable damping devices described above under Headings I-III and one or more therapeutic agents that target these factors. Some of these therapeutic agents are described above under Heading IV and include, but are not limited to, EDG Receptor Family Modulators, MMP inhibitors, and senolytic agents. When combined, the implantable damping devices and therapeutic agents of the present technology have a greater effect on treating and/or preventing one or more aspects of the condition upon a subject when compared either to the effects of the implantable damping device or therapeutic agent alone. For example, providing an implantable damping device that reduces the subject's pulse pressure and a therapeutic agent that reduces systemic inflammation and promotes BBB integrity, thereby treating and/or preventing progressive cognitive dysfunction and dementia. Subjects who have elevated pulse pressure or subjects that would otherwise benefit from having reduced pulse pressure may also have a blood brain barrier with increased permeability, one or more microbleeds, increased inflammation, increased oxidative stress, or other vascular dysfunction. The implantable damping device can be provided to these subjects having elevated pulse pressure or subjects that would otherwise benefit from having reduced pulse pressure before, after, or concurrently with a therapeutic agent, such as an EDG Receptor Family Modulator, MMP inhibitor, and senolytic agent.

Methods of the present disclosure include methods for treating a patient having a condition including the steps of, (a) determining or having determined whether the patient has an elevated pulse pressure or elevated pulse wave intensity/FCWI and a history of blood brain barrier dysregulation or permeability, decreasing reactive oxygen species (e.g., oxidative stress), decreasing microbleeds, decreasing inflammation (e.g., systemic inflammation, blood brain barrier inflammation, neuroinflammation), decreasing the level of at least one circulating or cerebral spinal fluid (CSF) cytokine, or a combination thereof by, (i) obtaining or having obtained information which indicates that the patient has the elevated pulse pressure or elevated pulse wave intensity/FCWI and has previously had symptoms of blood brain barrier dysregulation or permeability, oxidative stress, microbleeds, systemic inflammation, blood brain barrier inflammation, neuroinflammation, increased level of at least one circulating or CSF cytokine, or a combination thereof and/or was previously diagnosed with a microbleed, systemic inflammation, blood brain barrier inflammation, neuroinflammation, increased level of at least one circulating or CSF cytokine, or a combination thereof; and/or (ii) monitoring or having monitored the subject for the elevated pulse pressure or elevated pulse wave intensity/FCWI and symptoms of blood brain barrier dysregulation or permeability, oxidative stress, a microbleed, systemic inflammation, blood brain barrier inflammation, neuroinflammation, increased level of at least one circulating or CSF cytokine, or a combination thereof. In some embodiments, the methods also include, (b) if the patient has previously had symptoms of blood brain barrier dysregulation or permeability, oxidative stress, a microbleed, systemic inflammation, blood brain barrier inflammation, neuroinflammation, increased level of at least one circulating or CSF cytokine, or a combination thereof, was previously diagnosed with a microbleed, systemic inflammation, blood brain barrier inflammation, neuroinflammation, increased level of at least one circulating or CSF cytokine, or a combination thereof, and/or symptoms of blood brain barrier dysregulation or permeability, oxidative stress, a microbleed, systemic inflammation, blood brain barrier inflammation, neuroinflammation, increased level of at least one circulating or CSF cytokine, or a combination thereof were monitored, then (i) providing an EDG Receptor Family Modulator, an MMP inhibitor, or a senolytic agent to the patient, and (ii) providing a device for treating and/or preventing one or more effects of the condition. In some embodiments, the methods further include, (c) if the patient has not had symptoms of blood brain barrier dysregulation or permeability, oxidative stress, a microbleed, systemic inflammation, blood brain barrier inflammation, neuroinflammation, increased level of at least one circulating or CSF cytokine, or a combination thereof, was not previously diagnosed with blood brain barrier dysregulation or permeability, oxidative stress, a microbleed, systemic inflammation, blood brain barrier inflammation, neuroinflammation, increased level of at least one circulating or CSF cytokine, or a combination thereof, then providing the device for treating and/or preventing one or more effects of the condition.

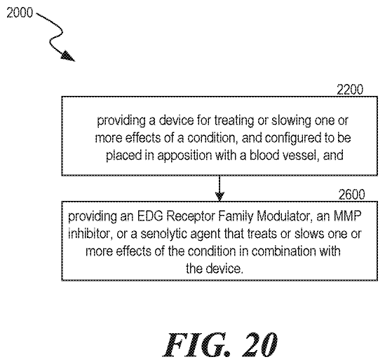

FIG. 20 is a flow chart illustrating method 2000 for treating and/or preventing one or more effects of a subject's condition. At block 2200, the method 2000 provides a device for treating and/or preventing one or more effects of the condition. The device is the implantable damping devices of the present technology and is configured to be placed in apposition with the subject's blood vessel. Similar to other devices of the present technology, the device provided in method 2000 includes the flexible damping member having both the inner surface formed of the sidewall having one or more at least partially deformable portions and the outer surface. In addition, the abating substance is disposed within the partially deformable portions and is configured to move longitudinally and/or radially therein in response to pulsatile blood flow within the blood vessel. At block 2600, the method 2000 provides an EDG Receptor Family Modulator, an MMP inhibitor, or a senolytic agent that treats or slows one or more effects of the condition in combination with the implantable damping device. In some embodiments, the EDG Receptor Family Modulator, MMP inhibitor, or senolytic agent is provided to the subject before the implantable damping device, up to about 24 hours, up to about 7 days, up to about 4 weeks, up to about 12 months, or up to about 5 years before the implantable damping device. In other embodiments, the implantable damping device is provided to the subject before the EDG Receptor Family Modulator, MMP inhibitor, or senolytic agent, up to about 24 hours, up to about 7 days, up to about 4 weeks, up to about 12 months, or up to about 5 years before the EDG Receptor Family Modulator, MMP inhibitor, or senolytic agent. For example, the EDG Receptor Family Modulator, MMP inhibitor, and/or the implantable damping device is provided to the subject about 0 to about 24 hours, about 1 to about 20 hours, about 3 to about 12 hours, about 5 to about 10 hours, about 1 day to about 7 days, about 2 days to about 6 days, about 3 days to about 5 days, about 1 week to about 4 weeks, about 2 weeks to about 4 weeks, about 1 week to about 3 weeks, about 2 weeks to about 3 weeks, about 1 year to about 5 years, about 1 year to about 4 years, about 2 years to about 5 years, about 2 years to about 4 years, about 3 years to about 4 years, or about 4 years to about 5 years before the implantable damping device, the EDG Receptor Family Modulator, MMP inhibitor, respectively.

Steps of the foregoing method and additional methods disclosed herein can be performed in any order. For example, step (b) is performed after step (a) and before step (c). As another example, step (c) is performed after step (a) and before step (b).

As described above under Heading IV, the EDG Receptor Family Modulator, MMP inhibitor, or senolytic agent of the methods of the present technology is provided to the subject by administration.

Devices useful with the methods of the present technology include implantable damping devices of the present disclosure, such as devices comprising a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and an abating substance disposed within the one or more at least partially deformable portions of the sidewall configured to move longitudinally and/or radially within one partially deformable portion in response to pulsatile blood flow within the blood vessel.

When combined with the implantable damping devices of the present technology, the therapeutic agents described herein are provided at a first dosage that is lower than a second dosage of the same therapeutic agents provided in the absence of the implantable damping devices (e.g., subjects receiving only the therapeutic agents rather than in combination with the implantable damping devices). For example, a subject having a neurodegenerative condition, such as dementia, is provided with a lower dose of Omni-Biotic before, during, or after being provided with the implantable damping device compared to a subject provided with a dose of Omni-Biotic without also being provided with the implantable damping device.

In some embodiments, when combined with the implantable damping devices of the present technology, the therapeutic agents described herein are provided with a first dosing regimen which is less than a second dosing regimen of the same therapeutic agents that is provided in the absence of the implantable damping devices. For example, a subject having a neurodegenerative condition, such as dementia, is provided with a first dosing regimen of Omni-Biotic before, during, or after being provided with the implantable damping device compared to a subject provided with a second dosing regimen of Omni-Biotic without also being provided with the implantable damping device.

In some embodiments, when combined with the implantable damping devices of the present technology, the therapeutic agents described herein are provided with the therapeutic agent by a first route which differs from a second route provided in the absence of the implantable damping devices. For example, a subject having a neurodegenerative condition, such as dementia, is provided with Omni-Biotic by the first route before, during, or after being provided with the implantable damping device compared to a subject provided with Omni-Biotic by the second route without also being provided with the implantable damping device. In some embodiments, the route of administration includes delivering the therapeutic agent to the subject from the device, for example, by eluting the therapeutic agent previously stored in at least a portion of the device.

### VI. SELECTED SYSTEMS FOR TREATING NEUROLOGICAL CONDITIONS WITH A COMBINATION OF AN IMPLANTABLE DAMPING DEVICE AND A THERAPEUTIC AGENT

In addition to the methods, damping devices, and therapeutic agents described herein, the present technology also includes associated systems for treating and/or preventing one or more effects of the subject's condition. Systems of the present technology include an effective amount of at least one therapy for treating and/or preventing one or more effects of the condition and a device for treating and/or preventing one or more effects of the condition. As explained above, devices of the present technology include at least a flexible damping member forming a generally tubular structure having an inner surface formed of a sidewall having one or more at least partially deformable portions, and an abating substance disposed within and configured to move longitudinally and/or radially within one partially deformable portion in response to pulsatile blood flow within the blood vessel. In some embodiments, the therapy includes at least one or more therapeutic agents that may be carried by the damping device. In these embodiments, the therapeutic agent is disposed within and/or carried by at least one or more of the at least partially deformable portions of the damping device. When one or more of the at least partially deformable portions of the damping device are at least partially deformed, the effective amount of the therapeutic agent may be released from the device.

### VII. EXAMPLES

The following examples are illustrative of several embodiments of the present technology. The embodiments described herein are directed to treatment regimens that include a damping device (e.g., any of the devices described above in paragraphs [93] to [136]) and a therapy (e.g., any EDG Receptor Family Modulator to include those discussed above in any of paragraphs [140] to [145]; any MMP inhibitor to include those discussed above in any of paragraphs [146] to [148]; and/or any senolytic agent to include those discussed above in any of paragraphs [149] to [151]). The treatment regimen is not limited to one therapy and may include one or more additional therapies. In accordance with some embodiments, the treatment regimen may be used in methods to treat or prevent a neurological condition using a combination therapy that includes steps of implanting or otherwise providing a damping device (e.g., any of the devices described above in paragraphs [93] to [136]) and administering or otherwise providing a therapy (e.g., any EDG Receptor Family Modulator to include those discussed above in any of paragraphs [140] to [145]; any MMP inhibitor to include those discussed above in any of paragraphs [146] to [148]; and/or any senolytic agent to include those discussed above in any of paragraphs [149] to [151]). In such embodiments, the damping device may be implanted or provided before the therapy, concurrently with the therapy, or after the therapy. One or more additional therapies may also be administered at any time during the combination therapy. Non-limiting examples of the damping device, treatments using the damping device, combination treatments and treatment regimens—as well as studies and models that demonstrate the efficacy, benefits, and advantages of the treatments—are included in the examples below.

### A. Examples 1-16: Devices for Treating and/or Preventing the Progression of Dementia

Example 1. A device for treating and/or preventing the progression of dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the arterial wall at the treatment site, thereby securing the damping member at the treatment site, and wherein the first and second anchoring members extend along only a portion of the length of the damping member such that at least a portion of the damping region is exposed between the first and second anchoring members and allowed to expand to a diameter greater than the deployed diameter.

Example 2. The device of example 1 wherein the damping member is configured to deform in response to a change in blood/pulse pressure or elevated pulse wave intensity/FCWI.

Example 3. The device of example 1 or example 2 wherein, at a location along the damping member coincident with a leading end of a pulse pressure wave, the distance between the inner surface and the outer surface of the damping member decreases in response to the pressure.

Example 4. The device of any one of examples 1-3 wherein the lumen of the damping member has an hourglass shape.

Example 5. The device of any one of examples 1˜4 wherein the outer surface is generally cylindrical and the inner surface is undulating.

Example 6. The device of any one of examples 1-5 wherein each of the first and second anchoring members is an expandable stent.

Example 7. The device of any one of examples 1-5 wherein the each of the first and second anchoring members is an expandable mesh.

Example 8. The device of any one of examples 1-5 wherein each of the first and second anchoring members is at least one of an expandable stent and an expandable mesh.

Example 9. The device of any one of examples 1-8 wherein each of the first and second anchoring members is positioned around a circumference of the damping member.

Example 10. The device of any one of examples 1-8 wherein at least a portion of each of the first and second anchoring members is positioned within the damping member and extends through at least a portion of the thickness of the sidewall.

Example 11. The device of any one of examples 1-10 wherein the damping region is a first damping region, and wherein the damping member includes a plurality of damping regions between the first and second end portions.

Example 12. The device of any one of examples 1-11 wherein at least one of the first and second anchoring members comprise a plurality of fixation devices extending radially outwardly from the outer surface of the damping device.

Example 13. The device of any one of examples 1-12 wherein the device is configured to be positioned at a treatment site within the left common carotid artery.

Example 14. The device of any one of examples 1-13 wherein the device is configured to be positioned at a treatment site within the right common carotid artery.

Example 15. The device of any one of examples 1-14 wherein the device is configured to treat Alzheimer's disease.

Example 16. The device of any one of examples 1-15 wherein the device is configured to reduce the occurrence of microbleeds in one or more branches of the artery downstream from the treatment site.

### B. Examples 17-31: Devices for Treating Dementia

Example 17. A device for treating dementia, comprising:
a damping member configured to be intravascularly positioned within an artery at a treatment site and having a lumen configured to direct blood flow to distal vasculature, the damping member being transformable between a low-profile state for delivery to the treatment site and an expanded state, wherein the damping member includes a damping region having a pressure limiter projecting laterally inwardly into the lumen to distribute pressure downstream from the damping member when a pulse pressure wave propagates along the damping member during systole; and
an anchoring member coupled to the damping member, wherein the anchoring member, in a deployed state, is configured to extend outwardly to a deployed diameter and contact a portion of the blood vessel wall at the treatment site, thereby securing the damping member at the treatment site, wherein the anchoring member extends along only a portion of the length of the damping member such that the damping region of the damping member is allowed to extend radially outward beyond the deployed diameter of the anchoring member.

Example 18. The device of example 17 wherein the damping member is configured to deform in response to a change in blood/pulse pressure or elevated pulse wave intensity/FCWI.

Example 19. The device of example 17 or example 18 wherein, at a location along the damping member coincident with a leading end of a pulse pressure wave, the distance between the inner surface and the outer surface of the damping member decreases in response to the pressure.

Example 20. The device of any one of examples 17-19 wherein the lumen of the damping member has an hourglass shape.

Example 21. The device of any one of examples 17-20 wherein the anchoring member is an expandable stent.

Example 22. The device of any one of examples 17-20 wherein the anchoring member is an expandable mesh.

Example 23. The device of any one of examples 17-20 wherein the anchoring member is at least one of an expandable stent and an expandable mesh.

Example 24. The device of any one of examples 17-23 wherein the anchoring member is positioned around a circumference of the damping member.

Example 25. The device of any one of examples 17-23 wherein at least a portion of the anchoring member is positioned within the damping member and extends through at least a portion of the thickness of the sidewall.

Example 26. The device of any one of examples 17-25 wherein the damping region is a first damping region, and wherein the damping member includes a plurality of damping regions between the first and second end portions.

Example 27. The device of any one of examples 17-26 wherein the anchoring member includes a plurality of fixation devices extending radially outwardly from the outer surface of the damping device.

Example 28. The device of any one of examples 17-27 wherein the device is configured to be positioned at a treatment site within the left common carotid artery.

Example 29. The device of any one of examples 17-28 wherein the device is configured to be positioned at a treatment site within the right common carotid artery.

Example 30. The device of any one of examples 17-29 wherein the device is configured to treat Alzheimer's disease.

Example 31. The device of any one of examples 17-29 wherein the device is configured to reduce the occurrence of microbleeds in portions of the blood vessel downstream from the treatment site.

### C. Example 32: Devices for Treating Dementia

Example 32. A device for treating dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the blood vessel wall at the treatment site, thereby securing the damping member at the treatment site, and wherein, when blood flows through the damping member during systole, the damping member absorbs a portion of the pulsatile energy of the blood, thereby reducing a magnitude of a pulse pressure transmitted to a portion of the blood vessel distal to the damping device.

### D. Examples 33-39: Devices for Treating a Blood Vessel

Example 33. A device for treating a blood vessel, comprising:
an anchoring system having a first portion and a second portion; and
a cushioning member located between the first and second portions of the anchoring system such that a portion of the cushioning member is not constrained by the anchoring system, and wherein the cushioning member is configured to absorb pulsatile energy transmitted by blood flowing with the vessel.

Example 34. The device of example 33 wherein the cushioning member is configured to expand in response to an increase of blood/pulse pressure or elevated pulse wave intensity/FCWI within the vessel, and relax as the blood/pulse pressure or elevated pulse wave intensity/FCWI within the vessel subsequently decreases.

Example 35. A device for treating a blood vessel, comprising:
an endovascular cushioning device having a proximal anchor and a distal anchor, each of the proximal and distal anchors being configured to abut against an inner wall of a major artery; and
an elastically deformable member extending between the proximal and distal anchors,
wherein the elastically deformable member is configured to expand in response to an increase of blood/pulse pressure or elevated pulse wave intensity/FCWI within the vessel, and relax as the blood/pulse pressure or elevated pulse wave intensity/FCWI within the vessel subsequently decreases.

Example 36. The device of example 35 wherein a portion of the elastically deformable membrane located longitudinally between the proximal and distal anchors defines a region of reduced internal cross-sectional area relative to the proximal and distal anchors when the elastically deformable membrane is radially relaxed.

Example 37. The device of example 35 or example 36 wherein the proximal and distal anchors are each radially expandable between a first diameter before deployment and a second diameter after deployment.

Example 38. The device of any one of examples 35-37, further comprising one or more threads secured to the proximal anchor.

Example 39. The device of example 38 wherein each thread is secured to an eyelet.

### E. Examples 40-43: Devices for Treating a Left Common Carotid Artery, a Right Common Carotid Artery, a Brachiocephalic Artery, the Ascending Aorta, an Internal Carotid Artery, or an Abdominal Aorta

Example 40. A device for treating an artery selected from a left common carotid artery, a right common carotid artery, a brachiocephalic artery, the ascending aorta, an internal carotid artery, or an abdominal aorta, the device comprising:
a wrap fabricated from an elastically deformable material, and
an engagement formation adapted to secure two opposing edges of the wrap around the artery,
wherein the elastically deformable material is configured to radially expand during a systole stage and radially contract during a diastole stage.

Example 41. The device of example 40 wherein the engagement formation includes sutures and/or staples.

Example 42. The device of example 41 wherein the engagement formation includes a zip lock.

Example 43. A device for treating a left common carotid artery, a right common carotid artery, a brachiocephalic artery, or an ascending aorta, the device comprising:
a proximal anchor configured to be wrapped around the artery;
a distal anchor configured to be wrapped around the artery and longitudinally spaced relative to the proximal anchor; and
a helical band adapted to be wound around the artery, the helical band having a first end securable to the proximal anchor and an opposing second end securable to the distal anchor, wherein the helical band is adapted to radially expand during a systole stage and radially contract during a diastole stage.

### F. Examples 44-85: Devices for Treating and/or Preventing the Effects of Dementia

Example 44. A device for treating and/or preventing the effects of dementia, comprising:
a damping member having a low-profile state and a deployed state, wherein, in the deployed state, the damping member comprises a deformable, generally tubular sidewall having an outer surface and an inner surface that is undulating in a longitudinal direction, and wherein the sidewall is configured to be positioned in apposition with a blood vessel wall to absorb pulsatile energy transmitted by blood flowing through the blood vessel.

Example 45. The device of example 44 wherein the damping member is configured to be positioned in apposition with at least one of a left common carotid artery, a right common carotid artery, and a brachiocephalic artery.

Example 46. The device of example 44 or example 45 wherein the damping member is configured to be positioned in apposition with an ascending aorta.

Example 47. The device of any one of examples 44-46 wherein the damping member is configured to be positioned in apposition with an inner surface of the blood vessel wall.

Example 48. The device of any one of examples 44-46 wherein the damping member is configured to be positioned in apposition with an outer surface of the blood vessel wall.

Example 49. The device of any one of examples 44-48 wherein the sidewall has an inner diameter, and, when the damping member is in a deployed state, the inner diameter increases then decreases in an axial direction.

Example 50. The device of any one of examples 44-49 wherein the cross-sectional area decreases then increases in longitudinal direction.

Example 51 The device of any one of examples 44-50 wherein the outer surface has a generally cylindrical shape.

Example 52. The device of any one of examples 44-50 wherein the outer surface has an undulating shape.

Example 53. The device of any one of examples 44-52, further comprising an anchoring member coupled to the damping member and axially aligned with only a portion of the damping member, wherein the anchoring member is configured to engage the blood vessel wall and secure the damping member to the blood vessel wall.

Example 54. The device of any one of examples 44-53 wherein the anchoring member is a first anchoring member and the device further comprises a second anchoring member coupled to the damping member, and wherein the second anchoring member:
is axially aligned with only a portion of the damping member, and
is spaced apart from the first anchoring member along the longitudinal axis of the damping member.

Example 55. The device of any one of examples 44-54 wherein, when the damping member is positioned adjacent the blood vessel wall, the damping member does not constrain the diameter of the blood vessel wall.

Example 56. A device for treating and/or preventing the effects of dementia, comprising:
an elastic member having a low-profile state for delivery to a treatment site at a blood vessel wall and a deployed state, wherein, in the deployed state, the elastic member is configured to abut an arterial wall and form a generally tubular structure having an inner diameter, an outer diameter, an outer surface, and an undulating inner surface, and wherein at least one of the outer diameter and the inner diameter increases and decreases in response to an increase and a decrease in pulse pressure within the blood vessel, respectively.

Example 57. The device of example 56 wherein the elastic member is configured to be positioned in apposition with at least one of a left common carotid artery, a right common carotid artery, and a brachiocephalic artery.

Example 58. The device of example 56 or example 57 wherein the elastic member is configured to be positioned in apposition with an ascending aorta.

Example 59. The device of any one of examples 56-58 wherein the elastic member is configured to be positioned in apposition with an inner surface of the blood vessel wall.

Example 60. The device of any one of examples 56-58 wherein the elastic member is configured to be positioned in apposition with an outer surface of the blood vessel wall.

Example 61. The device of any one of examples 56-60 wherein the sidewall has an inner diameter, and, when the elastic member is in a deployed state, the inner diameter increases then decreases in an axial direction.

Example 62. The device of any one of examples 56-61 wherein the cross-sectional area decreases then increases in longitudinal direction.

Example 63. The device of any one of examples 56-62 wherein the outer surface has a generally cylindrical shape.

Example 64. The device of any one of examples 56-62 wherein the outer surface has an undulating shape.

Example 65. The device of any one of examples 56-64, further comprising an anchoring member coupled to the elastic member and axially aligned with only a portion of the elastic member, wherein the anchoring member is configured to engage the blood vessel wall and secure the elastic member to the blood vessel wall.

Example 66. The device of example 65 wherein the anchoring member is a first anchoring member and the device further comprises a second anchoring member coupled to the elastic member, and wherein the second anchoring member:
is axially aligned with only a portion of the elastic member, and
is spaced apart from the first anchoring member along the longitudinal axis of the elastic member.

Example 67. The device of any one of examples 56-66 wherein, when the elastic member is positioned adjacent the blood vessel wall, the elastic member does not constrain the diameter of the blood vessel wall.

Example 68. A device for treating and/or preventing the effects of dementia, comprising:
a damping member including an abating substance, the damping member having a low-profile configuration and a deployed configuration, wherein, when the damping member is in the deployed configuration, the damping member forms a generally tubular structure configured to be positioned along the circumference of an artery such that, when a pulse wave traveling through the artery applies a stress at a first axial location along the length of the tubular structure, at least a portion of the abating substance moves away from the first location to a second axial location along the length of the tubular structure.

Example 69. The device of example 68, further comprising a structural element coupled to the damping member.

Example 70. The device of example 68 or example 69 wherein, in the deployed state, the damping member is configured to wrap around at least a portion of the circumference of the artery.

Example 71. The device of any one of examples 68-70 wherein, in the deployed state, the device has a pre-set helical configuration.

Example 72. The device of any one of examples 68-71 wherein the damping member includes a liquid.

Example 73. The device of any one of examples 68-72 wherein the damping member includes a gas.

Example 74. The device of any one of examples 68-73 wherein the damping member includes a gel.

Example 75. The device of any one of examples 68-74 wherein the damping member, in the deployed configuration, is configured to be positioned in apposition with an outer surface of the arterial wall.

Example 76. The device of any one of examples 68-74 wherein the damping member, in the deployed configuration, is configured to be positioned around the arterial wall such that an inner surface of the damping member is in contact with blood flowing through the artery.

Example 77. A device for treating and/or preventing the effects of dementia, comprising:
a damping member including a plurality of fluid particles, the damping member having a low-profile configuration and a deployed configuration, wherein, when the damping member is in the deployed configuration, the damping member is configured to be positioned along the circumference of an artery at a treatment site along a length of the artery,
wherein, when the damping member is in a deployed configuration and positioned at the treatment site, a wavefront traveling through the length of the artery redistributes at least a portion of the fluid particles along the length of the damping member such that the inner diameter of the damping member increases at the axial location along the damping member aligned with the wavefront while the inner diameter of the damping member at another axial location along the damping member decreases.

Example 78. The device of example 77, further comprising a structural element coupled to the damping member.

Example 79. The device of example 77 or example 78 wherein, in the deployed state, the damping member is configured to wrap around at least a portion of the circumference of the artery.

Example 80. The device of any one of examples 77-79 wherein, in the deployed state, the device has a pre-set helical configuration.

Example 81. The device of any one of examples 77-80 wherein the damping member includes a liquid.

Example 82. The device of any one of examples 77-81 wherein the damping member includes a gas.

Example 83. The device of any one of examples 77-82 wherein the damping member includes a gel.

Example 84. The device of any one of examples 77-83 wherein the damping member, in the deployed configuration, is configured to be positioned in apposition with an outer surface of the arterial wall.

Example 85. The device of any one of examples 77-84 wherein the damping member, in the deployed configuration, is configured to be positioned around the arterial wall such that an inner surface of the damping member is in contact with blood flowing through the artery.

### G. Examples 86-87: Methods for Treating and/or Preventing the Effects of Dementia

Example 86. A method for treating and/or preventing the effects of dementia, comprising:
positioning a damping device in apposition with at least one of the brachiocephalic artery, the right common carotid artery, the left common carotid artery, the ascending aorta, and the aortic arch, the damping device comprising an elastic, generally tubular sidewall whereby the damping device absorbs pulsatile energy transmitted by blood flowing through the at least one of the brachiocephalic artery, the right common carotid artery, the left common carotid artery, the ascending aorta, and the aortic arch.

Example 87. A method for treating and/or preventing the effects of dementia, comprising:
positioning a damping device in apposition with the wall of an artery that delivers blood to the brain, the damping device comprising an elastic, generally tubular sidewall having an outer surface and an undulating inner surface; and
in response to a pulse pressure wave in blood flowing through the blood vessel, a contour of at least one of the inner surface and the outer surface changes.

### H. Example 88: Methods for Treating at Least One of the Brachiocephalic Artery, the Right Common Carotid Artery, the Left Common Carotid Artery, the Ascending Aorta, and the Aortic Arch

Example 88. A method for treating at least one of the brachiocephalic artery, the right common carotid artery, the left common carotid artery, the ascending aorta, and the aortic arch, the method comprising:
positioning a damping device in apposition with a blood vessel wall, the damping device comprising an elastic, generally tubular sidewall;
expanding at least one of the inner diameter and the outer diameter of the damping device in response to an increase in pulse pressure; and
contracting at least one of the inner diameter and the outer diameter of the damping device in response to a decrease in pulse pressure.

### I. Examples 89-96: Methods of Treating a Blood Vessel

Example 89. A method of treating a blood vessel, comprising:
inserting a catheter into a vessel and directing a tip of the catheter to a desired vascular location;
transferring a distal anchor from within the catheter tip into the vessel;
expanding the distal anchor such that a radially outer portion of the distal anchor engages with an inner wall of the vessel;
withdrawing the catheter slightly and transferring a proximal anchor from the tip of the catheter into the vessel;
longitudinally positioning the proximal anchor at a desired location;
expanding the proximal anchor such that a radially outer portion of the proximal anchor engages with an inner wall of the vessel, wherein an elastically deformable member extends longitudinally between the proximal and distal anchors.

Example 90. The method of example 89 wherein transferring the distal anchor includes advancing the distal anchor from the tip of the catheter.

Example 91. The method of example 89 or example 90 wherein transferring the distal anchor includes withdrawing the tip of the catheter whilst the distal anchor remains at a generally constant longitudinal position within the vessel, and exits from the tip of the catheter.

Example 92. The method of any one of examples 89-91 wherein longitudinally positioning the proximal anchor includes applying a first tensile force to one or more threads frangibly secured to the proximal anchor.

Example 93. The method of example 92, further including frangibly rupturing the thread(s) after expanding the proximal anchor by applying a second tensile force which is greater than the first tensile force.

Example 94. The method of example 92, further including disengaging a ring, latch or clasp secured to the thread(s) after expanding the proximal anchor in order to disengage the thread from the proximal anchor.

Example 95. The method of any one of examples 89-94, further including imaging to determine the location of the proximal and/or distal anchors.

Example 96. A method of treating a blood vessel selected from a left common carotid artery, a right common carotid artery or a brachiocephalic artery, a carotid artery, a branch of any of the foregoing, and an ascending aorta, the method comprising:
wrapping an elastically deformable material around the artery; and
attaching a first edge of the elastically deformable material to an opposing second edge of the elastically deformable material such that an internal diameter of the elastically deformable material is smaller than an initial outer diameter of the artery during a systole stage.

### J. Examples 97-113: Methods for Treating Dementia

Example 97. A method for treating dementia, comprising:
intravascularly positioning a damping device within an artery at a treatment site, wherein the damping device includes an anchoring member coupled to an elastic, tubular damping member defining a lumen therethrough;
expanding the anchoring member and the damping member from a low-profile state to an expanded state such that at least the anchoring member is in apposition with the arterial wall at the treatment site; and
changing a contour of the damping member in response to a pulse pressure wave in blood flow through the damping member.

Example 98. The method of example 97, further comprising reducing a magnitude of the pulse pressure transmitted to a portion of the blood vessel distal to the damping device.

Example 99. The method of example 98 wherein reducing a magnitude of the pulse pressure includes absorbing a portion of the pulsatile energy of blood flowing through the artery.

Example 100. The method of any one of examples 97-99 wherein changing a contour of the damping member includes increasing an inner diameter of the lumen damping member while an outer diameter of the damping member remains generally constant.

Example 101. The method of any one of examples 97-99 wherein changing a contour of the damping member includes increasing an inner diameter and an outer diameter of the lumen of the damping member.

Example 102. The method of any one of examples 97-99 wherein changing a contour of the damping member includes decreasing a distance between an inner surface of the damping member and an outer surface of the damping member.

Example 103. The method of example 97-102 wherein intravascularly positioning a damping device includes intravascularly positioning a damping device within a left common carotid artery at a treatment site.

Example 104. The method of any one of examples 97-103 wherein intravascularly positioning a damping device includes intravascularly positioning a damping device within a right common carotid artery at a treatment site.

Example 105. The method of any one of examples 97-104 wherein expanding the anchoring member and expanding the damping member occurs simultaneously.

Example 106. The method of any one of examples 97-105 wherein expanding the anchoring member includes expanding the anchoring member with a balloon.

Example 107. The method of any one of examples 97-105 wherein expanding the anchoring member includes withdrawing a sheath to expose the anchoring member to allow the anchoring member to self-expand.

Example 108. The method of any one of examples 97-107 wherein expanding the damping member includes expanding the damping member with a balloon.

Example 109. The method of any one of examples 97-107 wherein expanding the damping member includes withdrawing a sheath to expose the damping member to allow the anchoring member to self-expand.

Example 110. The method of any one of examples 97-109 wherein expanding the anchoring member forces the damping member to expand.

Example 111. The method of any one of examples 97-110 wherein:
the damping device is a first damping device,
the first damping device is intravascularly positioned at a first arterial location, and
the method further comprises intravascularly positioning a second damping device at a second arterial location different than the first arterial location.

Example 112. The method of example 111 wherein the first arterial location is one of a left common carotid artery, a right common carotid artery, an external carotid artery, an internal carotid artery, and an ascending aorta, and the second arterial location is one of a left common carotid artery, a right common carotid artery, an external carotid artery, an internal carotid artery, and an ascending aorta.

Example 113. The method of example 111 wherein the first arterial location is a left common carotid artery and the second arterial location is a right common carotid artery.

### K. Examples 114-115: Method for Treating and/or Preventing the Effects of Dementia

Example 114. A method for treating and/or preventing the effects of dementia, comprising:
positioning a damping member along a length of an artery, the damping member including an abating substance; and
in response to a pulse wave traveling through blood in the artery, redistributing at least a portion of the abating compound along the length of the damping member, thereby attenuating at least a portion of the energy of the pulse wave in the blood.

Example 115. A method for treating and/or preventing the effects of dementia, comprising:
positioning a damping member along a length of an artery, the damping member including a plurality of fluid particles; and
moving a portion of the fluid particles away from an axial location along the damping member aligned a wavefront of a pulse wave, thereby increasing the inner diameter of the damping member.

### L. Examples 116-131: Devices for Treating and/or Preventing the Progression of Dementia

Example 116. A device for treating and/or preventing the progression of dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the arterial wall at the treatment site, thereby securing the damping member at the treatment site, and wherein the first and second anchoring members extend along only a portion of the length of the damping member such that at least a portion of the damping region is exposed between the first and second anchoring members and allowed to expand to a diameter greater than the deployed diameter.

Example 117. The device of example 116 wherein the damping member is elastically deformable, and is configured to deform in response to a change in blood/pulse pressure or elevated pulse wave intensity/FCWI.

Example 118. The device of example 116 or example 117 wherein, at a location along the damping member coincident with a leading end of a pulse pressure wave, the distance between the inner surface and the outer surface of the damping member decreases in response to the pressure.

Example 119. The device of any one of examples 116-118 wherein the lumen of the damping member has an hourglass shape.

Example 120. The device of any one of example 116-119 wherein the outer surface is generally cylindrical and the inner surface is undulating.

Example 121. The device of any one of examples 116-120 wherein each of the first and second anchoring members is an expandable stent.

Example 122. The device of any one of examples 116-120 wherein the each of the first and second anchoring members is an expandable mesh.

Example 123. The device of any one of examples 116-120 wherein each of the first and second anchoring members is at least one of an expandable stent and an expandable mesh.

Example 124. The device of any one of examples 116-123 wherein each of the first and second anchoring members is positioned around a circumference of the damping member.

Example 125. The device of any one of examples 116-124 wherein at least a portion of each of the first and second anchoring members is positioned within the damping member and extends through at least a portion of the thickness of the sidewall.

Example 126. The device of any one of examples 116-125 wherein the damping region is a first damping region, and wherein the damping member includes a plurality of damping regions between the first and second end portions.

Example 127. The device of any one of examples 116-126 wherein at least one of the first and second anchoring members comprise a plurality of fixation devices extending radially outwardly from the outer surface of the damping device.

Example 128. The device of any one of examples 116-127 wherein the device is configured to be positioned at a treatment site within the left common carotid artery.

Example 129. The device of any one of examples 116-127 wherein the device is configured to be positioned at a treatment site within the right common carotid artery.

Example 130. The device of any one of examples 116-129 wherein the device is configured to treat Alzheimer's disease.

Example 131. The device of any one of examples 116-129 wherein the device is configured to reduce the occurrence of microbleeds in one or more branches of the artery downstream from the treatment site.

### M. Examples 132-147: Devices for Treating Dementia

Example 132. A device for treating dementia, comprising:
a damping member configured to be intravascularly positioned within an artery at a treatment site and having a lumen configured to direct blood flow to distal vasculature, the damping member being transformable between a low-profile state for delivery to the treatment site and an expanded state, wherein the damping member includes a damping region having a pressure limiter projecting laterally inwardly into the lumen to distribute pressure downstream from the damping member when a pulse pressure wave propagates along the damping member during systole; and
an anchoring member coupled to the damping member, wherein the anchoring member, in a deployed state, is configured to extend outwardly to a deployed diameter and contact a portion of the blood vessel wall at the treatment site, thereby securing the damping member at the treatment site, wherein the anchoring member extends along only a portion of the length of the damping member such that the damping region of the damping member is allowed to extend radially outward beyond the deployed diameter of the anchoring member.

Example 133. The device of example 132 wherein the damping member is elastically deformable, and is configured to deform in response to a change in blood/pulse pressure or elevated pulse wave intensity/FCWI.

Example 134. The device of example 132 or 133 wherein, at a location along the damping member coincident with a leading end of a pulse pressure wave, the distance between the inner surface and the outer surface of the damping member decreases in response to the pressure.

Example 135. The device of any one of examples 132-134 wherein the lumen of the damping member has an hourglass shape.

Example 136. The device of any one of examples 132-135 wherein the anchoring member is an expandable stent.

Example 137. The device of any one of examples 132-136 wherein the anchoring member is an expandable mesh.

Example 138. The device of any one of examples 132-137 wherein the anchoring member is at least one of an expandable stent and an expandable mesh.

Example 139. The device of any one of examples 132-138 wherein the anchoring member is positioned around a circumference of the damping member.

Example 140. The device of any one of examples 132-139 wherein at least a portion of the anchoring member is positioned within the damping member and extends through at least a portion of the thickness of the sidewall.

Example 141. The device of any one of examples 132-140 wherein the damping region is a first damping region, and wherein the damping member includes a plurality of damping regions between the first and second end portions.

Example 142. The device of any one of examples 132-141 wherein the anchoring member includes a plurality of fixation devices extending radially outwardly from the outer surface of the damping device.

Example 143. The device of any one of examples 132-142 wherein the device is configured to be positioned at a treatment site within the left common carotid artery.

Example 144. The device of any one of examples 132-142 wherein the device is configured to be positioned at a treatment site within the right common carotid artery.

Example 145. The device of any one of examples 132-144 wherein the device is configured to treat Alzheimer's disease.

Example 146. The device of any one of examples 132-145 wherein the device is configured to reduce the occurrence of microbleeds in portions of the blood vessel downstream from the treatment site.

Example 147. A device for treating dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the blood vessel wall at the treatment site, thereby securing the damping member at the treatment site, and
wherein, when blood flows through the damping member during systole, the damping member absorbs a portion of the pulsatile energy of the blood, thereby reducing a magnitude of a pulse pressure transmitted to a portion of the blood vessel distal to the damping device.

### N. Examples 148-154: Devices for Treating a Blood Vessel

Example 148. A device for treating a blood vessel, comprising:
an anchoring system having a first portion and a second portion which is spaced apart from the first portion in a first direction; and
a cushioning member located between the first and second portions of the anchoring system such that movement of a portion of the cushioning member in a second direction, which is orthogonal to the first direction, is not constrained by the anchoring system, and wherein the cushioning member is configured to absorb pulsatile energy transmitted by blood flowing with the vessel.

Example 149. The device of example 148 wherein the cushioning member is elastically deformable and is configured to expand in response to an increase of blood/pulse pressure or elevated pulse wave intensity/FCWI within the vessel and relax as the blood/pulse pressure or elevated pulse wave intensity/FCWI within the vessel subsequently decreases.

Example 150. A device for treating a blood vessel, comprising:
an endovascular cushioning device having a proximal anchor and a distal anchor which is spaced apart from the proximal anchor, each of the proximal and distal anchors being configured to abut against an inner wall of a major artery; and
an elastically deformable member extending between the proximal and distal anchors,
wherein the elastically deformable member is configured to expand in response to an increase of blood/pulse pressure or elevated pulse wave intensity/FCWI within the vessel and relax as the blood/pulse pressure or elevated pulse wave intensity/FCWI within the vessel subsequently decreases.

Example 151. The device of example 150 wherein a portion of the elastically deformable membrane located longitudinally between the proximal and distal anchors defines a region of reduced internal cross-sectional area relative to the proximal and distal anchors when the elastically deformable membrane is radially relaxed.

Example 152. The device of example 150 or example 151 wherein the proximal and distal anchors are each radially expandable between a first diameter before deployment and a second diameter after deployment.

Example 153. The device of any one of examples 150-152, further comprising one or more threads secured to the proximal anchor.

Example 154. The device of example 153 wherein each thread is secured to an eyelet.

### O. Examples 155-160: Devices for Treating an Artery Selected from a Left Common Carotid Artery, a Right Common Carotid Artery, a Brachiocephalic Artery, the Ascending Aorta, an Internal Carotid Artery, or an Abdominal Aorta

Example 155. A device for treating an artery selected from a left common carotid artery, a right common carotid artery, a brachiocephalic artery, the ascending aorta, an internal carotid artery, or an abdominal aorta, the device comprising:
a wrap fabricated from an elastically deformable material, and
an engagement formation adapted to secure two opposing edges of the wrap around the artery,
wherein the elastically deformable material is configured to radially expand during a systole stage and radially contract during a diastole stage.

Example 156. The device of example 155 wherein, when the wrap is in position around the artery, the wrap entirely or substantially entirely surrounds the artery over a portion of its length.

Example 157. The device of example 155 wherein the engagement formation includes sutures and/or staples.

Example 158. The device of example 155 wherein the engagement formation includes a zip lock.

Example 159. A device for treating a left common carotid artery, a right common carotid artery, a brachiocephalic artery, or an ascending aorta, the device comprising:
a proximal anchor configured to be wrapped around the artery;
a distal anchor configured to be wrapped around the artery and longitudinally spaced relative to the proximal anchor; and
a helical band adapted to be wound around the artery, the helical band having a first end securable to the proximal anchor and an opposing second end securable to the distal anchor, wherein the helical band is adapted to radially expand during a systole stage and radially contract during a diastole stage.

Example 160. The device of example 159 wherein the first end of the helical band is secured to the proximal anchor and the second end of the helical band is secured to the distal anchor.

### P. Examples 161-214: Devices for Treating and/or Preventing the Effects of Dementia

Example 161. A device for treating and/or preventing the effects of dementia, comprising:
a damping member comprising a deformable, generally tubular sidewall having an outer surface and an inner surface that is undulating in a longitudinal direction, and wherein the sidewall is configured to be positioned in apposition with a blood vessel wall to absorb pulsatile energy transmitted by blood flowing through the blood vessel.

Example 162. The device of example 161 wherein the damping member is configured to be positioned in apposition with at least one of a left common carotid artery, a right common carotid artery, and a brachiocephalic artery.

Example 163. The device of example 161 wherein the damping member is configured to be positioned in apposition with an ascending aorta.

Example 164. The device of any one of examples 161-163 wherein the damping member is configured to be positioned in apposition with an inner surface of the blood vessel wall.

Example 165. The device of any one of examples 161-163 wherein the damping member is configured to be positioned in apposition with an outer surface of the blood vessel wall.

Example 166. The device of any one of examples 161-165 wherein the sidewall has an inner diameter, and, when the damping member is in a deployed state, the inner diameter increases then decreases in an axial direction.

Example 167. The device of any one of examples 161-166 wherein the cross-sectional area decreases then increases in longitudinal direction.

Example 168. The device of any one of examples 161-167 wherein the outer surface has a generally cylindrical shape.

Example 169. The device of any one of examples 161-167 wherein the outer surface has an undulating shape.

Example 170. The device of any one of examples 161-169, further comprising an anchoring member coupled to the damping member and axially aligned with only a portion of the damping member, wherein the anchoring member is configured to engage the blood vessel wall and secure the damping member to the blood vessel wall.

Example 171. The device of example 170 wherein the anchoring member is a first anchoring member and the device further comprises a second anchoring member coupled to the damping member, and wherein the second anchoring member:
is axially aligned with only a portion of the damping member, and
is spaced apart from the first anchoring member along the longitudinal axis of the damping member.

Example 172. The device of any one of examples 161-171 wherein, when the damping member is positioned adjacent the blood vessel wall, the damping member does not constrain the diameter of the blood vessel wall.

Example 173. A device for treating and/or preventing the effects of dementia, comprising:
an elastic member which is configured to abut an arterial wall and form a generally tubular structure having an inner diameter, an outer diameter, an outer surface, and an undulating inner surface, and wherein at least one of the outer diameter and the inner diameter increases and decreases in response to an increase and a decrease in pulse pressure within the blood vessel, respectively.

Example 174. The device of example 173 wherein the elastic member is configured to be positioned in apposition with at least one of a left common carotid artery, a right common carotid artery, and a brachiocephalic artery.

Example 175. The device of example 173 wherein the elastic member is configured to be positioned in apposition with an ascending aorta.

Example 176. The device of any one of examples 173-175 wherein the elastic member is configured to be positioned in apposition with an inner surface of the blood vessel wall.

Example 177. The device of any one of examples 173-175 wherein the elastic member is configured to be positioned in apposition with an outer surface of the blood vessel wall.

Example 178. The device of any one of examples 173-177 wherein the sidewall has an inner diameter, and, when the elastic member is in a deployed state, the inner diameter increases then decreases in an axial direction.

Example 179. The device of any one of examples 173-178 wherein the cross-sectional area decreases then increases in longitudinal direction.

Example 180. The device of any one of examples 173-179 wherein the outer surface has a generally cylindrical shape.

Example 181. The device of any one of examples 173-179 wherein the outer surface has an undulating shape.

Example 182. The device of any one of examples 173-181, further comprising an anchoring member coupled to the elastic member and axially aligned with only a portion of the elastic member, wherein the anchoring member is configured to engage the blood vessel wall and secure the elastic member to the blood vessel wall.

Example 183. The device of example 182 wherein the anchoring member is a first anchoring member and the device further comprises a second anchoring member coupled to the elastic member, and wherein the second anchoring member:
is axially aligned with only a portion of the elastic member, and
is spaced apart from the first anchoring member along the longitudinal axis of the elastic member.

Example 184. The device of any one of examples 173 to 183 wherein, when the elastic member is positioned adjacent the blood vessel wall, the elastic member does not constrain the diameter of the blood vessel wall.

Example 185. The device of any one of examples 173 to 184 wherein the damping member or elastic member has a low-profile state and a deployed state.

Example 186. The device of example 185 wherein the deployed state is for delivery to a treatment site at a blood vessel wall.

Example 187. The device of example 185 or 186 wherein the damping member or elastic member has a first, lesser outer diameter when in the low-profile state and a second, greater diameter when in the deployed state.

Example 188. A device for treating and/or preventing the effects of dementia, comprising:
a damping member including an abating substance, wherein the damping member forms a generally tubular structure having an axis, wherein the abating substance is able to move axially relative to the tubular structure, and wherein the damping member is configured to be positioned along the circumference of an artery such that, when a pulse wave traveling through the artery applies a stress at a first axial location along the length of the tubular structure, at least a portion of the abating substance moves away from the first location to a second axial location along the length of the tubular structure.

Example 189. The device of example 188, wherein the abating substance comprises a quantity of a fluid and/or gel comprising particles, contained within a flexible member, and the particles may move axially relative to the tubular structure within the flexible member.

Example 190. The device of example 189 wherein the flexible member may, at least some locations along the length of the tubular structure, be deformed radially with respect to the tubular structure.

Example 191. The device of any one of examples 188-190, further comprising a structural element coupled to the damping member.

Example 192. The device of any one of examples 188-191 wherein, in a deployed state, the damping member is configured to wrap around at least a portion of the circumference of the artery.

Example 193. The device of example 192 wherein the damping member includes a break along its length, to allow it to be fitted around the portion of the circumference of the artery.

Example 194. The device of example 193, further comprising cooperating sealing arrangements located on or near opposing edges of the break, to allow the edges to be joined together once the damping member has been fitted around the portion of the circumference of the artery.

Example 195. The device of any one of examples 188-194 wherein, in a deployed state, the device has a pre-set helical configuration.

Example 196. The device of any one of examples 188-195 wherein the damping member includes a liquid.

Example 197. The device of any one of examples 188-196 wherein the damping member includes a gas.

Example 198. The device of any one of examples 188-197 wherein the damping member includes a gel.

Example 199. The device of any one of examples 188-198 wherein the damping member, in a deployed configuration, is configured to be positioned in apposition with an outer surface of the arterial wall.

Example 200. The device of any one of examples 188-199 wherein the damping member, in a deployed configuration, is configured to be positioned around the arterial wall such that an inner surface of the damping member is in contact with blood flowing through the artery.

Example 201. A device for treating and/or preventing the effects of dementia, comprising:
wherein the fluid particles are able to move axially along at least a part of the length of the damping structure, the damping member being configured to be positioned along the circumference of an artery at a treatment site along a length of the artery,
wherein, when the damping member is in a deployed configuration and positioned at the treatment site, a wave front traveling through the length of the artery redistributes at least a portion of the fluid particles along the length of the damping member such that the inner diameter of the damping member increases at the axial location along the damping member aligned with the wave front while the inner diameter of the damping member at another axial location along the damping member decreases.

Example 202. The device of example 201 wherein the fluid particles are contained within a flexible member, and the particles may move along the length of the damping member within the flexible member.

Example 203. The device of example 202 wherein the flexible member may, at least some locations along the length of the damping member, be deformed radially with respect to the damping member.

Example 204. The device of any one of examples 201-203, further comprising a structural element coupled to the damping member.

Example 205. The device of any one of examples 201-204 wherein, in the deployed state, the damping member is configured to wrap around at least a portion of the circumference of the artery.

Example 206. The device of example 205 wherein the damping member includes a break along its length, to allow it to be fitted around the portion of the circumference of the artery.

Example 207. The device of example 206, further comprising cooperating sealing arrangements located on or near opposing edges of the break, to allow the edges to be joined together once the damping member has been fitted around the portion of the circumference of the artery.

Example 208. The device of any one of examples 201-207 wherein, in the deployed state, the device has a pre-set helical configuration.

Example 209. The device of any one of examples 201-208 wherein the damping member includes a liquid.

Example 210. The device of any one of examples 201-209 wherein the damping member includes a gas.

Example 211. The device of any one of examples 201-210 wherein the damping member includes a gel.

Example 212. The device of any one of examples 201-211 wherein the damping member, in the deployed configuration, is configured to be positioned in apposition with an outer surface of the arterial wall.

Example 213. The device of any one of examples 201-212 wherein the damping member, in the deployed configuration, is configured to be positioned around the arterial wall such that an inner surface of the damping member is in contact with blood flowing through the artery.

Example 214. The device of any one of examples 201-213 wherein the damping member has a low-profile configuration and a deployed configuration.

### Q. Examples 215-401: Methods for Treating and/or Preventing One or More Effects of a Condition

Example 215. A method for treating and/or preventing one or more effects of a condition in a subject in need thereof, the method comprising:
providing a device for treating and/or preventing one or more effects of the condition, and configured to be placed in apposition with a blood vessel, the device comprising—
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel; and
providing a therapy that treats or slows one or more effects of the condition in combination with the device.

Example 216. The method of example 215, wherein the therapy is an EDG Receptor Family Modulator, an MMP inhibitor, or a senolytic agent or combination thereof.

Example 217. The method of example 216, wherein the EDG Receptor Family Modulator is an S1P receptor agonist selected from fingolimod, siponimod, ozanimod, or ponesimod.

Example 218. The method of example 216, wherein the MMP inhibitor is doxycycline or dexamethasone.

Example 219. The method of example 216, wherein the senolytic agent is dasatinib, quercetin, or a combination of dasatinib and quercetin.

Example 220. The method of any one of examples 215 to 219, wherein the therapy prevents and/or reduces abnormal cleavage of amyloid precursor protein in the subject's brain, prevents and/or reduces expression and/or accumulation of β-amyloid protein in the subject's brain, reduces and/or prevents oxidative stress, reduces and/or prevents ischemia, prevents dysregulation/damage and/or death of a neuron, prevents dysregulation and/or damage to the blood brain barrier, eliminates senescent cells, improves tight junctions, inhibits MMPs, and/or induces transcriptional activation of TIMPs.

Example 221. The method of any one of examples 215 to 220, wherein the therapy is provided at a first dosage which is lower than a second dosage that is provided in the absence of the device.

Example 222. The method of any one of examples 215 to 235, wherein the therapy is provided at a first dosing regimen which is less than a second dosing regimen that is provided in the absence of the device.

Example 223. The method of any one of examples 215 to 236, wherein the therapy is provided via a first route which is different than a second route that is provided in the absence of the device.

Example 224. The method of any one of examples 215 to 235, wherein the therapy is provided by administering the therapy to the subject in need thereof.

Example 225. The method of any one of examples 215 to 222, wherein the condition is neurodegeneration.

Example 226. The method of example 225, wherein neurodegeneration further comprises Alzheimer's disease, dementia, and/or cognitive impairment.

Example 227. The method of any one of examples 215 to 226, wherein the device has a low-profile state and a deployed state, and when in the deployed state, the sidewall is generally tubular.

Example 228. The method of any one of examples 215 to 227, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 229. The method of example 228, wherein when positioned in apposition with the blood vessel and a pulse wave travels through the blood vessel, the flexible damping member applies a stress at the first location along a length of the tubular structure.

Example 230. The method of example 229, wherein, after stress is applied at the first location, at least the portion of the abating substance moves longitudinally and/or radially along a length of the tubular structure.

Example 231. The method of example 229, wherein, after stress is applied at the first location, at least a portion of the abating substance is configured to move longitudinally and/or radially from a first location within a first deformable portion to a second location within the first deformable portion of the flexible damping member.

Example 232. The method of example 229, wherein, after stress is applied at the first location, at least the portion of the abating substance is further configured to move longitudinally and/or radially from the first location to a third location within a second deformable portion of the flexible damping member.

Example 233. The method of any one of examples 215 to 232, wherein the inner surface and/or an outer surface has a generally cylindrical shape or an undulating shape that undulates in a longitudinal direction.

Example 234. The method of any one of examples 215 to 233, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 235. The method of any one of examples 215 to 234, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 236. The method of example 235, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 237. A method for treating and/or preventing one or more effects of a condition in a subject in need thereof, the method comprising:
providing a device for treating and/or preventing one or more effects of the condition and configured to be placed in apposition with a blood vessel, the device comprising—
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel; and
wherein a therapy that treats or slows one or more effects of the condition has previously been provided to the subject in need thereof.

Example 238. The method of example 237, wherein the therapy is EDG Receptor Family Modulator, an MMP inhibitor, or a senolytic agent or combination thereof.

Example 239. The method of example 238, wherein the EDG Receptor Family Modulator is an S1P receptor agonist selected from fingolimod, siponimod, ozanimod, or ponesimod.

Example 240. The method of example 238, wherein the MMP inhibitor is doxycycline or dexamethasone.

Example 241. The method of example 238, wherein the senolytic agent is dasatinib, quercetin, or a combination of dasatinib and quercetin.

Example 241. The method of any one of examples 237 to 240, wherein the therapy prevents and/or reduces abnormal cleavage of amyloid precursor protein in the subject's brain, prevents and/or reduces expression and/or accumulation of β-amyloid protein in the subject's brain, reduces and/or prevents oxidative stress, reduces and/or prevents ischemia, prevents or reduces dysregulation/damage and/or death of a neuron, prevents or reduces blood brain barrier dysregulation or permeability/damage, eliminates senescent cells, improves tight junctions, inhibits MMPs, and/or induces transcriptional activation of TIMPs.

Example 242. The method of any one of examples 237 to 241, wherein the therapy is provided at a first dosage which is lower than a second dosage that is provided in the absence of the device.

Example 243. The method of any one of examples 237 to 242, wherein the therapy is provided at a first dosing regimen which is less than a second dosing regimen that is provided in the absence of the device.

Example 244. The method of any one of examples 237 to 243, wherein the therapy is provided via a first route which is different than a second route that is provided in the absence of the device.

Example 245. The method of any one of examples 237 to 244, wherein the therapy is provided by administering the therapy to the subject in need thereof.

Example 246. The method of any one of examples 237 to 244, wherein the condition is neurodegeneration.

Example 247. The method of example 246, wherein neurodegeneration further comprises Alzheimer's disease, dementia, and/or cognitive impairment.

Example 248. The method of any one of examples 237 to 247, wherein the device has a low-profile state and a deployed state, and when in the deployed state, the sidewall is generally tubular.

Example 249. The method of any one of examples 237 to 248, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 250. The method of example 249, wherein when positioned in apposition with the blood vessel and a pulse wave travels through the blood vessel, the flexible damping member applies a stress at the first location along a length of the tubular structure.

Example 251. The method of example 250, wherein, after stress is applied at the first location, at least the portion of the abating substance moves longitudinally and/or radially along a length of the tubular structure.

Example 252. The method of example 250, wherein, after stress is applied at the first location, at least a portion of the abating substance is configured to move longitudinally and/or radially from a first location within a first deformable portion to a second location within the first deformable portion of the flexible damping member.

Example 253. The method of example 250, wherein, after stress is applied at the first location, at least the portion of the abating substance is further configured to move longitudinally and/or radially from the first location to a third location within a second deformable portion of the flexible damping member.

Example 254. The method of any one of examples 237 to 253, wherein the inner surface and/or an outer surface has a generally cylindrical shape or an undulating shape that undulates in a longitudinal direction.

Example 255. The method of any one of examples 237 to 254, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 256. The method of any one of examples 237 to 255, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 257. The method of example 256, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 258. A method for treating and/or preventing one or more effects of a condition in a subject in need thereof, the method comprising:
providing a therapy for treating and/or preventing one or more effects of the condition to the subject in need thereof, wherein the subject has previously been provided a device that treats or slows one or more effects of the condition and was placed in apposition with a blood vessel, the device comprising—
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel.

Example 259. The method of example 258, wherein the therapy is an EDG Receptor Family Modulator, an MMP inhibitor, or a senolytic agent or combination thereof.

Example 260. The method of example 259, wherein the EDG Receptor Family Modulator is an S1P receptor agonist selected from fingolimod, siponimod, ozanimod, or ponesimod.

Example 261. The method of example 260, wherein the MMP inhibitor is doxycycline or dexamethasone.

Example 262. The method of example 260, wherein the senolytic agent is dasatinib, quercetin, or a combination of dasatinib and quercetin.

Example 263. The method of any one of examples 258 to 262, wherein the therapy, prevents and/or reduces abnormal cleavage of amyloid precursor protein in the subject's brain, prevents and/or reduces expression and/or accumulation of β-amyloid protein in the subject's brain, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, eliminates senescent cells, improves tight junctions, inhibits MMPs, and/or induces transcriptional activation of TIMPs.

Example 264. The method of any one of examples 258 to 263, wherein the therapy is provided at a first dosage which is lower than a second dosage that is provided in the absence of the device.

Example 265. The method of any one of examples 258 to 264, wherein the therapy is provided at a first dosing regimen which is less than a second dosing regimen that is provided in the absence of the device.

Example 266. The method of any one of examples 258 to 265, wherein the therapy is provided via a first route which is different than a second route that is provided in the absence of the device.

Example 267. The method of any one of examples 258 to 266, wherein the therapy is provided by administering the therapy to the subject in need thereof.

Example 268. The method of any one of examples 258 to 267, wherein the condition is neurodegeneration.

Example 269. The method of example 268, wherein neurodegeneration further comprises Alzheimer's disease, dementia, and/or cognitive impairment.

Example 270. The method of any one of examples 258 to 269, wherein the device has a low-profile state and a deployed state, and when in the deployed state, the sidewall is generally tubular.

Example 271. The method of any one of examples 258 to 270, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 272. The method of example 271, wherein when positioned in apposition with the blood vessel and a pulse wave travels through the blood vessel, the flexible damping member applies a stress at the first location along a length of the tubular structure.

Example 273. The method of example 272, wherein, after stress is applied at the first location, at least the portion of the abating substance moves longitudinally and/or radially along a length of the tubular structure.

Example 274. The method of example 273, wherein, after stress is applied at the first location, at least a portion of the abating substance is configured to move longitudinally and/or radially from a first location within a first deformable portion to a second location within the first deformable portion of the flexible damping member.

Example 275. The method of example 274, wherein, after stress is applied at the first location, at least the portion of the abating substance is further configured to move longitudinally and/or radially from the first location to a third location within a second deformable portion of the flexible damping member.

Example 276. The method of any one of examples 258 to 275, wherein the inner surface and/or an outer surface has a generally cylindrical shape or an undulating shape that undulates in a longitudinal direction.

Example 277. The method of any one of examples 258 to 276, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 278. The method of any one of examples 258 to 277, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 279. The method of example 278, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 280. A system for treating and/or preventing one or more effects of a condition in a subject in need thereof, the system comprising:
an effective amount of a therapy for treating and/or preventing one or more effects of the condition
a device for treating and/or preventing one or more effects of the condition,
the device comprising—
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel.

Example 281. The system of example 280, wherein the EDG Receptor Family Modulator, an MMP inhibitor, or a senolytic agent or combination thereof.

Example 282. The system of example 281, wherein the EDG Receptor Family Modulator is an S1P receptor agonist selected from fingolimod, siponimod, ozanimod, or ponesimod.

Example 283. The system of example 282, wherein the MMP inhibitor is doxycycline or dexamethasone.

Example 284. The system of example 282, wherein the senolytic agent is dasatinib, quercetin, or a combination of dasatinib and quercetin.

Example 285. The method of any one of examples 280 to 284, wherein the therapy prevents and/or reduces abnormal cleavage of amyloid precursor protein in the subject's brain, prevents and/or reduces expression and/or accumulation of β-amyloid protein in the subject's brain, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, eliminates senescent cells, improves tight junctions, inhibits MMPs, and/or induces transcriptional activation of TIMPs.

Example 286. The system of any one of examples 280 to 285, wherein the therapy is provided at a first dosage which is lower than a second dosage that is provided in the absence of the device.

Example 287. The system of any one of examples 280 to 286, wherein the therapy is provided at a first dosing regimen which is less than a second dosing regimen that is provided in the absence of the device.

Example 288. The system of any one of examples 280 to 287, wherein the therapy is provided via a first route which is different than a second route that is provided in the absence of the device.

Example 289. The system of any one of examples 280 to 288, wherein the therapy is provided by administering the therapy to the subject in need thereof.

Example 290. The system of any one of examples 280 to 289, wherein the condition is neurodegeneration.

Example 291. The system of example 290, wherein neurodegeneration further comprises Alzheimer's disease, dementia, and/or cognitive impairment.

Example 292. The system of any one of examples 280 to 291, wherein the inner surface and/or an outer surface has a generally cylindrical shape or an undulating shape that undulates in a longitudinal direction.

Example 293. The system of any one of examples 280 to 292, wherein the device has a low-profile state and a deployed state, and when in the deployed state, the sidewall is generally tubular.

Example 294. The system of any one of examples 280 to 293, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 295. The system of example 294, wherein when positioned in apposition with the blood vessel and a pulse wave travels through the blood vessel, the flexible damping member applies a stress at the first location along a length of the tubular structure.

Example 296. The system of example 295, wherein, after stress is applied at the first location, at least the portion of the abating substance moves longitudinally and/or radially along a length of the tubular structure.

Example 297. The system of example 295, wherein, after stress is applied at the first location, at least a portion of the abating substance is configured to move longitudinally and/or radially from a first location within a first deformable portion to a second location within the first deformable portion of the flexible damping member.

Example 298. The system of example 295, wherein, after stress is applied at the first location, at least the portion of the abating substance is further configured to move longitudinally and/or radially from the first location to a third location within a second deformable portion of the flexible damping member.

Example 299. The system of any one of examples 280 to 298, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 300. The system of any one of examples 280 to 299, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 301. The system of example 300, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 302. A system for treating and/or preventing one or more effects of a condition in a subject in need thereof, the system comprising:
an effective amount of a therapy for treating and/or preventing one or more effects of the condition
a device for treating and/or preventing one or more effects of the condition,
the device comprising—
   a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
   an abating substance disposed within the one or more at least partially deformable portions of the sidewall configured to move longitudinally and/or radially within one partially deformable portion in response to pulsatile blood flow within the blood vessel;
wherein, the effective amount of the therapy for treating and/or preventing one or more effects of the condition is carried by or more of the at least partially deformable portions of the device, and
wherein, when the one or more at least partially deformable portions are at least partially deformed, the effective amount of therapy for treating and/or preventing one or more effects of the condition is released from the device.

Example 303. The system of example 302, wherein the effective amount of the therapy further comprises a first effective amount of the therapy and a second effective amount of the therapy.

Example 304. The system of example 303, wherein the second effective amount of the therapy is greater than the first effective amount of the therapy.

Example 305. The system of example 304, wherein, in response to a first pulsatile blood flow within the blood vessel, the one or more at least partially deformable portions are at least partially deformed to a first degree of deformation.

Example 306. The system of example 305, wherein, in response to a second pulsatile blood flow within the blood vessel, the one or more at least partially deformable portions are at least partially deformed to a second degree of deformation.

Example 307. The system of example 306, wherein the second degree of deformation is greater than the first degree of deformation.

Example 308. The system of example 307, wherein the first effective amount of the therapy is released from the one or more at least partially deformable portions in response to the first degree of deformation.

Example 309. The system of example 308, wherein the second effective amount of the therapy is released from the one or more at least partially deformable portions in response to the second degree of deformation.

Example 310. The system of any one of examples 302 to 309, wherein the therapy is an EDG Receptor Family Modulator, an MMP inhibitor, or a senolytic agent or combination thereof.

Example 311. The system of example 310, wherein the EDG Receptor Family Modulator is an S1P receptor agonist selected from fingolimod, siponimod, ozanimod, or ponesimod.

Example 312. The system of example 310, wherein the MMP inhibitor is doxycycline or dexamethasone.

Example 313. The system of example 310, wherein the senolytic agent is dasatinib, quercetin, or a combination of dasatinib and quercetin.

Example 314. The method of any one of examples 359 to 384, wherein the therapy prevents and/or reduces abnormal cleavage of amyloid precursor protein in the subject's brain, prevents and/or reduces expression and/or accumulation of β-amyloid protein in the subject's brain, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, eliminates senescent cells, improves tight junctions, inhibits MMPs, and/or induces transcriptional activation of TIMPs.

Example 315. The system of any one of examples 359 to 385, wherein the therapy is provided at a first dosage which is lower than a second dosage that is provided in the absence of the device.

Example 316. The system of any one of examples 359 to 386, wherein the therapy is provided at a first dosing regimen which is less than a second dosing regimen that is provided in the absence of the device.

Example 317. The system of any one of examples 359 to 387, wherein the therapy is provided via a first route which is different than a second route that is provided in the absence of the device.

Example 318. The system of any one of examples 359 to 388, wherein the therapy is provided by administering the therapy to the subject in need thereof.

Example 319. The system of any one of examples 359 to 389, wherein the condition is neurodegeneration.

Example 320. The system of example 390, wherein neurodegeneration further comprises Alzheimer's disease, dementia, and/or cognitive impairment.

Example 321. The system of any one of examples 359 to 391, wherein the inner surface and/or an outer surface has a generally cylindrical shape or an undulating shape that undulates in a longitudinal direction.

Example 322. The system of any one of examples 359 to 392, wherein the device has a low-profile state and a deployed state, and when in the deployed state, the sidewall is generally tubular.

Example 323. The system of any one of examples 359 to 393, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 324. The system of example 394, wherein when positioned in apposition with the blood vessel and a pulse wave travels through the blood vessel, the flexible damping member applies a stress at the first location along a length of the tubular structure.

Example 325. The system of example 395 wherein, after stress is applied at the first location, at least the portion of the abating substance moves longitudinally and/or radially along a length of the tubular structure.

Example 326. The system of example 395, wherein, after stress is applied at the first location, at least a portion of the abating substance is configured to move longitudinally and/or radially from a first location within a first deformable portion to a second location within the first deformable portion of the flexible damping member.

Example 327. The system of example 395, wherein, after stress is applied at the first location, at least the portion of the abating substance is further configured to move longitudinally and/or radially from the first location to a third location within a second deformable portion of the flexible damping member.

Example 328. The system of any one of examples 359 to 398, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 329. The system of any one of examples 359 to 399, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 330. The system of example 400, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

### R. Examples 331-349: Methods for Treating a Patient Having a Condition

Example 331. A method for treating a patient having a condition comprising:
(a) determining or having determined whether the patient has an elevated pulse pressure, elevated pulse wave intensity/FCWI, and a history of a microbleed, blood brain barrier dysregulation and/or increased permeability, systemic inflammation, blood brain barrier inflammation, neuroinflammation, increased level of at least one circulating or cerebral spinal fluid (CSF) cytokine, increased level of reactive oxygen species, or a combination thereof by:
   (i) obtaining or having obtained information which indicates that the patient has the elevated pulse pressure, elevated pulse wave intensity/FCWI, and has previously had symptoms of a microbleed, blood brain barrier dysregulation and/or increased permeability, systemic inflammation, blood brain barrier inflammation, neuroinflammation, increased level of at least one circulating or CSF cytokine, reactive oxygen species, or a combination thereof and/or was previously diagnosed with a microbleed, blood brain barrier dysregulation and/or increased permeability, systemic inflammation, blood brain barrier inflammation, neuroinflammation, increased level of at least one circulating or CSF cytokine, increased reactive oxygen species, or a combination thereof; and/or
   (ii) monitoring or having monitored the subject for the elevated pulse pressure, elevated pulse wave intensity/FCWI, and symptoms of a microbleed, blood brain barrier dysregulation and/or increased permeability, systemic inflammation, blood brain barrier inflammation, neuroinflammation, increased level of at least one circulating cytokine, increased reactive oxygen species, or a combination thereof; and
(b) if the patient has previously had symptoms of a microbleed, blood brain barrier dysregulation and/or increased permeability, systemic inflammation, blood brain barrier inflammation, neuroinflammation, increased level of at least one circulating cytokine, increased reactive oxygen species, or a combination thereof, was previously diagnosed with a microbleed, blood brain barrier dysregulation and/or increased permeability, systemic inflammation, blood brain barrier inflammation, neuroinflammation, increased level of at least one circulating cytokine, increased reactive oxygen species, or a combination thereof, and/or symptoms of a microbleed, systemic inflammation, blood brain barrier inflammation, neuroinflammation, increased level of at least one circulating cytokine, or a combination thereof were monitored, then
   (i) providing an EDG Receptor Family Modulator, an MMP inhibitor, or a senolytic agent, or combination thereof to the patient, and
   (ii) providing a device for treating and/or preventing one or more effects of the condition,
the device comprising—
   a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
   an abating substance disposed within the one or more at least partially deformable portions of the sidewall configured to move longitudinally and/or radially within one partially deformable portion in response to pulsatile blood flow within the blood vessel;
(c) if the patient has not had symptoms of a microbleed, blood brain barrier dysregulation and/or increased permeability, systemic inflammation, blood brain barrier inflammation, neuroinflammation, increased level of at least one circulating cytokine, increased reactive oxygen species, or a combination thereof, was not previously diagnosed with a microbleed, blood brain barrier dysregulation and/or increased permeability, systemic inflammation, blood brain barrier inflammation, neuroinflammation, increased level of at least one circulating cytokine, increased reactive oxygen species or a combination thereof, then providing the device for treating and/or preventing one or more effects of the condition.

Example 332. The method of example 331, wherein the elevated pulse pressure is a pulse pressure of at least 50 mmHg.

Example 333. The method of example 331 or example 332, wherein the increased level of at least one circulating cytokine in (b) is a level at least about 5% greater than the level in (c).

Example 334. The method of any one of examples 331 to 333, wherein the at least one cytokine is selected from the group consisting of VCAM-1, ICAM-1, TNFα, TGF-β, IL-6, IL-8, IL-1β, IL-12, and NF-κB.

Example 335. The method of example 331, wherein the EDG Receptor Family Modulator is an S1P receptor agonist selected from fingolimod, siponimod, ozanimod, or ponesimod.

Example 336. The method of example 331, wherein the MMP inhibitor is doxycycline or dexamethasone.

Example 337. The method of example 331, wherein the senolytic agent is dasatinib, quercetin, or a combination of dasatinib and quercetin.

Example 338. The method of any one of examples 331 to 337, wherein the therapy prevents and/or reduces abnormal cleavage of amyloid precursor protein in the subject's brain, prevents and/or reduces expression and/or accumulation of β-amyloid protein in the subject's brain, decreases inflammation, reduces and/or prevents oxidative stress, reduces and/or prevents ischemia, prevents or reduces dysfunction/damage and/or death of a neuron, prevents or reduces blood brain barrier dysregulation and/or increase permeability, eliminates senescent cells, improves tight junctions, inhibits MMPs, and/or induces transcriptional activation of TIMPs.

Example 339. The method of any one of examples 331 to 338, wherein the therapy is provided at a first dosage which is lower than a second dosage that is provided in the absence of the device.

Example 340. The method of any one of examples 331 to 339, wherein the therapy is provided at a first dosing regimen which is less than a second dosing regimen that is provided in the absence of the device.

Example 341. The method of any one of examples 331 to 340, wherein the therapy is provided via a first route which is different than a second route that is provided in the absence of the device.

Example 342. The method of any one of examples 331 to 341, wherein the therapy is provided by administering the therapy to the subject in need thereof.

Example 343. The method of any one of examples 331 to 342, wherein step (b) is performed after step (a) and before step (c).

Example 344. The method of any one of examples 331 to 343, wherein step (c) is performed after step (a) and before step (b).

Example 345. The method of any one of examples 331 to 344, wherein the condition is neurodegeneration.

Example 346. The method of example 345, wherein neurodegeneration further comprises Alzheimer's disease, dementia, and/or cognitive impairment.

Example 347. The method of any one of examples 331 to 346, wherein the inner surface and/or an outer surface has a generally cylindrical shape or an undulating shape that undulates in a longitudinal direction.

Example 348. The method of any one of examples 331 to 347, wherein the device has a low-profile state and a deployed state, and when in the deployed state, the sidewall is generally tubular.

Example 349. The method of any one of examples 331 to 348, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

In addition, the following prophetic examples are illustrative of several embodiments of the present technology.

### S. Example 350: Clinical Study Design (Prophetic Example)

Implantable devices will be positioned at, near, around, within, or in place of at least a portion of a subject's artery in accordance with the present technology. After the implantable devices have been positioned, subjects who received the implantable device will be randomized into one of at least two groups: Group A—placebo, and Group B—therapeutic agent. The placebo will be an experimentally appropriate placebo useful for distinguishing any specific effects of the drug, such as the pharmaceutically acceptable carrier for the active component in the therapeutic agent. The dose of the placebo will be comparable to the amount of pharmaceutically acceptable carrier that subjects in Group B receive. Group B can include two or more subgroups, with subjects being randomly assigned to each subgroup. While the subjects in each of these Group B subgroups each ultimately receive the same therapeutic agent, the dose, route of administration, dosing regimen, or other parameters associated with a therapeutic protocol can be altered.

### T. Example 351: Clinical Study Design (Prophetic Example)

A therapeutic agent will be delivered to a subject at a pre-specified dose, route of administration, frequency, and duration. After the therapeutic agent has been delivered to the subject, subjects will be randomized into one of at least two groups: Group A—sham, and Group B—implantable device. For those subjects in Group B, implantable devices will be positioned at, near, around, within, or in place of at least a portion of a subject's artery in accordance with the present technology. The sham treatment for Group A includes the delivery methods associated with delivery of the implantable device used for Group B, although the implantable device will not be delivered to the subjects in Group A.

In an alternative study, subjects will be randomized into one of at least two groups: Group A—sham, and Group B—implantable device. For those subjects in Group B, implantable devices will be positioned at, near, around, within, or in place of at least a portion of a subject's artery in accordance with the present technology. The sham treatment for Group A includes the delivery methods associated with delivery of the implantable device used for Group B, although the implantable device will not be delivered to the subjects in Group A. After Groups A and B have been selected and implantable devices (Group B) or sham treatments (Group A) have been provided, a therapeutic agent will be delivered to a subject at a pre-specified dose, route of administration, frequency, and duration.

In another alternative study, subjects will be randomized into one of at least two groups: Group A—sham, and Group B—implantable device. For those subjects in Group B, implantable devices will be positioned at, near, around, within, or in place of at least a portion of a subject's artery in accordance with the present technology, and a therapeutic agent will be delivered to the subjects at a pre-specified dose, route of administration, frequency, and duration at the time of implantation. The sham treatment for Group A includes the delivery methods associated with delivery of the implantable device used for Group B, although the implantable device will not be delivered to the subjects in Group A. Group A will also be delivered a therapeutic agent at a pre-specified dose, route of administration, frequency, and duration at the time of sham treatment.

### U. Example 352: Agent-Based Therapy Study (Prophetic Example)

Influence of Reduced Pulsatility on Agent-Based Therapy: Immortalized human cerebral microvascular endothelial cells, such as HCMEC-SV40 cells, will be cultured and cyclically stretched ˜15%, as described in Gangoda et al, 2018. After the cerebral endothelial cells have been cyclically stretched, confluence will be noted, and samples of conditioned media will be collected. Half of the cerebral endothelial cell cultures will be reduced to ˜5% stretch, while the other half will remain at 15% stretch. This will represent +/−application of the device described herein, respectively.

Next, combination therapy with a drug (MMP inhibitor, (e.g., dexamethasone, doxycycline), S1P modulator (e.g., fingolimod), or senolytic therapy (e.g., D+Q) will be administered as follows:
(I) Cerebral endothelial cells reduced to 5% stretch+agent
(II) Cerebral endothelial cells maintained at 15% stretch+agent.

At a predetermined time after drug dosing, the following assays will be performed for each condition:
(I) Conditioned medium will be collected, and ELISAs will be used to measure the expression of relevant biomarkers such as MMPs, S1P receptors, inflammatory cytokines, and amyloid pathway proteins (normalized to confluence).
(II) Cell viability may be quantified using an LDH assay.
(III) Western blots of cerebral microvascular endothelial cells will be used to quantify cellular content of inflammatory and amyloid pathway proteins.

### V. Example 353: In Vivo Surgically Induced Animal Model Study Design (Prophetic Example)

In Vivo Model of Device. Male WT mice (e.g., C57Bl/6 and BALB/c) and/or Alzheimer disease model mice will be acclimatized for 2 weeks prior to the experiment. Animals will be kept under a standard condition with room temperature at 21-23° C., 30-70% relative humidity, and a 12:12 h light:dark cycle. Chow and water are available ad libitum.

After 2 weeks of acclimatization, mice will be weighed (weekly throughout the full study). Right carotid and left carotid blood flow and pulse pressure will be measured using any standard method in the art, e.g., a non-invasive Doppler Flow Velocity System (Scintica Instrumentation, Inc.), or invasive pressure catheter (Millar). Mice will then split into groups with comparable average weights. Mice will undergo transverse aortic constriction (TAC) surgery, which involves placing a constriction around the transverse aorta, limiting left ventricular (LV) outflow—thus creating a pressure overload in the LV and inducing an elevated pulse pressure. The TAC surgery will be performed using either (i) a nylon suture to induce a sustained (or permanent) elevated pulse pressure, or (ii) an absorbable suture (e.g., Polyglactin 910, which lasts 14-21 days at maximum strength) to induce a transient (or temporary) elevated pulse pressure, which will gradually dissipate as the suture dissolves, representing the reduction of elevated pulse pressure and associated cerebral microbleeds that occurs over time over time. The specific absorption rate of the selected absorbable suture and its effect on pulse pressure over time in mice will be confirmed. The TAC mice receiving the absorbable suture models the damping device.

Next, the following groups of mice will be examined to confirm that less Alzheimer-related pathology/more blood brain barrier (BBB) integrity is seen in the absorbable suture mice as compared to the nylon suture TAC mice, and to support the damping device's mechanism of action (reducing elevated pulse pressure).
(I) WT mice, TAC with nylon suture
(II) WT mice, TAC with absorbable suture
   AND/OR
(III) Alzheimer mice, TAC with nylon suture
(IV) Alzheimer mice, TAC with absorbable suture

The time point for group comparisons after the TAC procedure may be determined based on when optimal, contrasting pulse pressures are achieved with nylon versus absorbable sutures. In certain aspects, the experiment start point may be approximately 6 weeks post-TAC.

At the time point for group comparisons, mice will be weighed, and blood flow and pulse pressure will be recorded. Mice will then be injected with Evans blue dye (excitation 470/540 nm, emission 680 nm) or any other fluorescent tracer known in the art, undergo perfusion, then sacrificed. Whole brains will be extracted, and brain tissue will immediately be fixed (or snap frozen depending on what the tissue will be used for) and sectioned. Mouse hearts may be collected and weighed. Fixed ipsilateral and contralateral histological brain sections will be stained with Prussian Blue to quantify microbleeds as described in Supplement 1 of Montgolfier et al. (see above) or confocal imaged for fluorescence distribution. Immunohistochemistry or qPCR to measure expression of BBB markers such as tight junction proteins may also be considered as an indicator of BBB integrity. MMP (a marker for increased BBB permeability) and NF-kB (an inflammatory marker associated with Alzheimer's disease) protein expression may also be measured in brain microvessels and brain tissue. Amyloid-beta deposition in neural tissue may be quantified in Alzheimer's model mice as well. Finally, immunohistochemistry of fibrin in brain sections may be conducted to visualize clotting of blood components that have leaked through the BBB into the neuropil.

In Vivo Model of Combination Therapy. Following successful completion of the proof-of-concept experiments described above, the benefit of reducing elevated pulse pressure on the efficacy of drug or cell therapies will be studied in vivo. First, as detailed previously, WT or Alzheimer's model mice will undergo TAC surgery with either a nylon suture or absorbable suture.

At the time point for group comparison after the TAC procedure discussed above, the mice will be dosed (via intracarotid or tail vein) with fluorescently labeled stem/progenitor cells or a drug described herein (e.g., a “therapeutic agent”, “agent”, “therapy”, EDG Receptor Family Modulator, MMP inhibitor, senolytic agent and/or combination thereof).

For Example, the mice may be dosed with a therapeutic agent (MMP inhibitor, (e.g., dexamethasone, doxycycline), S1P modulator (e.g., fingolimod), or senolytic therapy (e.g., D+Q) as shown below:
(I) TAC with nylon suture+therapeutic agent
(II) TAC with absorbable suture+therapeutic agent

Exemplary therapies that may be tested according to this example include, but are not limited to, fingolimod, dexamethasone, and doxycycline.

At a time point for group comparisons, mice will be weighed, and blood flow and pulse pressure will be recorded. Mice will then be injected with Evans blue dye/fluorescent tracer, undergo perfusion, then sacrificed. Whole brains will be extracted, and brain tissue will immediately be fixed (or snap frozen depending on what the tissue will be used for) and sectioned. Mouse hearts will be collected and weighed. Fixed ipsilateral and contralateral histological brain sections will be stained with Prussian Blue to quantify microbleeds, see Supplement 1 of Montgolfier et al. (see above), or confocal imaged for fluorescence distribution. The PKH26/CFDA-SE (fluorescence) signal may reveal abundance of recruited fluorescence-labeled stem or progenitor cells in cerebral microvasculature. Evans blue/fluorescent tracer signal may show BBB leakage. Immunohistochemistry or qPCR to measure expression of BBB markers such as tight junction proteins could also be considered as an indicator of BBB integrity. MMP (a marker for increased BBB permeability) and NF-kB (an inflammatory marker associated with Alzheimer's disease and stem cell dysfunction) protein expression could also be measured in brain tissue. Finally, immunohistochemistry of fibrin in brain sections could be conducted to visualize clotting of blood components that have leaked through the BBB into the neuropil.

The in vivo model described herein may be used to test any therapy administered in combination with reduction of elevated pulse pressure (effect of the damping device), including the therapies discussed herein. These combination therapy experiments will confirm whether the absorbable suture TAC mice+therapeutics show less disrupted BBB than the nylon suture TAC mice+therapeutics. If so, this will support the potential for the device to synergistically enhance the efficacy of such therapeutics.

### W. Example 354: In Vivo Chemically Induced Pulse Pressure Study Design (Prophetic Example)

As an alternative to the TAC surgically induced model of elevated pulse pressure discussed above, a chemically induced animal model of elevated pulse pressure may also be used to test biological therapeutics described herein (e.g., a “therapeutic agent”, “agent”, “therapy”, EDG Receptor Family Modulator, an MMP inhibitor, and/or a senolytic agent or combination thereof). Such models can be generated by treating the animals (e.g., rats) with warfarin/vitamin K1 (WVK) in accordance with Essalihi et al., A new model of isolated systolic hypertension induced by chronic warfarin and vitamin K1 treatment, Am J Hypertens. 2003 February; 16(2): 103-10 (doi: 10.1016/s0895-7061(02)03204-1). The WVK treatment induces isolated systolic hypertension and thus, elevated pulse pressure. This elevated pulse pressure can be reduced with subsequent treatment with darusentan (DAR) and/or acetazolamide (ACTZ) treatment in accordance with Essalihi et al., Regression of medial elastocalcinosis in rat aorta: a new vascular function for carbonic anhydrase, Circulation. 2005 Sep. 13; 112(11):1628-35 (doi: 10.1161/CIRCULATIONAHA, 104.528984).

To compare cerebral vascular health animals experiencing elevated pulse pressure compared to when that pulse pressure is reduced, animals will be treated in one or more of the groups that follow:

| Treatment Group | Treatment | Duration |
|---|---|---|
| Maintained elevated pulse | WVK treatment | 8 weeks (or other suitable |
| pressure arm | | predetermined duration) |
| Reduced elevated pulse | WVK treatment | 4 weeks (or other suitable |
| pressure arm | DAR and/or ACTZ | predetermined duration) |
| | treatment | 4 weeks (or other suitable |
| | | predetermined duration) |
| Maintained elevated pulse | WVK treatment | 8 weeks (or other suitable |
| pressure + Drug arm | EDG Receptor Family | predetermined duration) |
| | Modulator treatment | 4 weeks (or other suitable |
| | | predetermined duration) |
| Maintained elevated pulse | WVK treatment | 8 weeks (or other suitable |
| pressure + Drug arm | MMP inhibitor treatment | predetermined duration) |
| | | 4 weeks (or other suitable |
| | | predetermined duration) |
| Maintained elevated pulse | WVK treatment | 8 weeks (or other suitable |
| pressure + Drug arm | senolytic agent treatment | predetermined duration) |
| | | 4 weeks (or other suitable |
| | | predetermined duration) |
| Reduced elevated pulse | WVK treatment | 4 weeks (or other suitable |
| pressure + Drug arm | DAR and/or ACTZ | predetermined duration) |
| | treatment + EDG Receptor | 4 weeks (or other suitable |
| | Family Modulator | predetermined duration) |
| Reduced elevated pulse | WVK treatment | 4 weeks (or other suitable |
| pressure + Drug arm | DAR and/or ACTZ | predetermined duration) |
| | treatment + MMP inhibitor | 4 weeks (or other suitable |
| | treatment | predetermined duration) |
| Reduced elevated pulse | WVK treatment | 4 weeks (or other suitable |
| pressure + Drug arm | DAR and/or ACTZ | predetermined duration) |
| | treatment + senolytic | 4 weeks (or other suitable |
| | agent treatment | predetermined duration) |

After the treatment period, cognition, microbleeds, BBB permeability, neuroinflammation, neurodegeneration, and expression of BBB tight junctions could be quantified and compared for each group of rats detailed in the table above

The study design described herein may be used to test any therapy intended to be administered in combination with reduction of elevated pulse pressure (effect of the damping device), including the therapies described herein. Exemplary therapies treatments that may be tested using this study design include, but are not limited to, fingolimod, dexamethasone, and doxycycline.

### X. Example 355: Fingolimod can Improve BBB Function in the Presence of Persistently High Pulse Pressure

This study was performed to investigate a “combined” approach to protecting the Blood Brain Barrier (BBB) by (i) a drug approach, in this case Fingolimod; and (ii) an experimental approach to mimic the effect of a device designed to reduce cerebral pulse pressure. Here, the experimental approach was the application of a resorbable suture to the ascending aorta (TAC procedure) between the right and left common carotid arteries. A permanent TAC produces high pulse pressure in the right cerebral hemisphere, while a transient TAC (resorbable suture) should mimic a period of high pulse pressure followed by normal pulse pressure once the suture is absorbed, similar to that which should be obtained with an External Carotid Artery device, as described in the embodiments herein.

Here, a resorbable TAC suture was used, which the manufacturer represented that would resorb after 7-10 days, however, in all but one mouse, resorption did not occur as discussed below. But, this experiment still gave us the opportunity to test if Fingolimod improved BBB function in the presence of persistently high pulse pressure which is a useful “proof of concept” experiment for the combined drug and device approach.

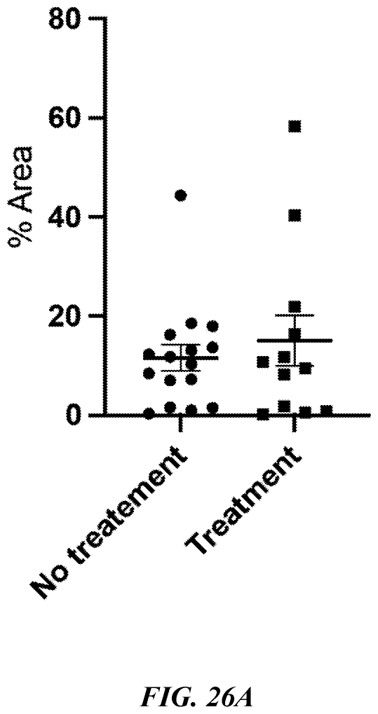

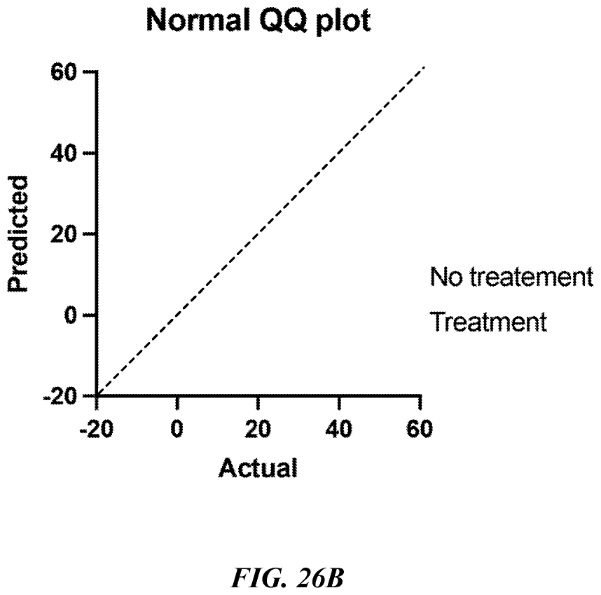

Study Design. 60 mice were divided evenly into two groups: a permanent TAC group receiving a plain suture, and a transient TAC group receiving a resorbable suture. As shown in FIG. 26, the protocol calls for treating all mice with fingolimod staring from day 12 post TAC surgery. Deviation from that protocol is discussed below.

Mortality rate after TAC. The mortality rate of mice in the permanent TAC group (7.4%) was higher than that of the transient TAC group (33.3%). The absorbable suture thread is not as rigid, and it took a few surgeries to adjust the tension applied to the suture.

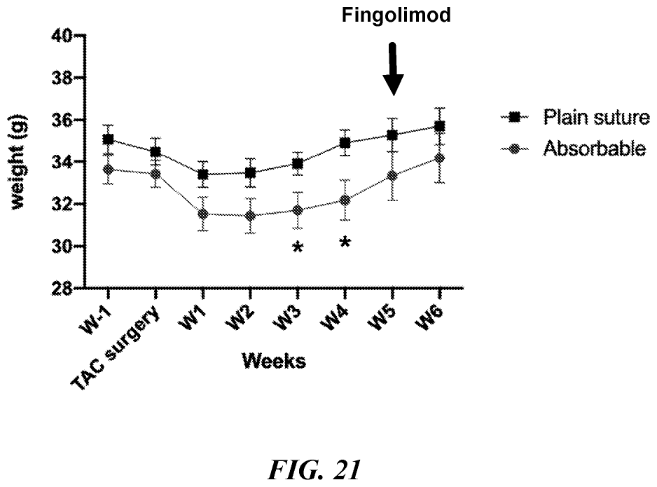

Body weight follow-up after TAC. As shown in FIG. 21 and Tables 2-3 below, TAC surgery with the absorbable suture was associated with a higher weight loss.

TABLE 2
Plain Suture: body weight 1 week before TAC surgery
(W-1) to six (6) weeks after TAC surgery (W6)

| | | TAC | | | | | | |
|---|---|---|---|---|---|---|---|---|
| | W-1 | surgery | W1 | W2 | W3 | W4 | W5 | W6 |
| Mean | 35.08 | 34.48 | 33.4 | 33.48 | 33.91 | 34.91 | 35.29 | 35.71 |
| Std. Dev | 3.475 | 3.355 | 3.096 | 3.417 | 2.627 | 2.953 | 3.635 | 3.567 |
| Std. Error of Mean | 0.6815 | 0.6458 | 0.6191 | 0.6834 | 0.5478 | 0.6157 | 0.7993 | 0.865 |
| No. of Values (n) | 26 | 27 | 25 | 25 | 23 | 23 | 21 | 17 |

TABLE 3
Absorbable Suture: body weight 1 week before TAC surgery
(W-1A) to six (6) weeks after TAC surgery (W6A)

| | | TAC | | | | | | |
|---|---|---|---|---|---|---|---|---|
| | W-1A | surgery | W1A | W2A | W3A | W4A | W5A | W6A |
| Mean | 33.64 | 33.42 | 31.52 | 31.43 | 31.7 | 32.17 | 33.33 | 34.19 |
| Std. Dev | 3.936 | 3.691 | 3.871 | 3.941 | 4.072 | 4.549 | 4.982 | 4.75 |
| Std. Error of Mean | 0.6851 | 0.5426 | 0.8073 | 0.8217 | 0.8492 | 0.9486 | 1.174 | 1.188 |
| No. of Values (n) | 33 | 33 | 23 | 23 | 23 | 23 | 18 | 16 |

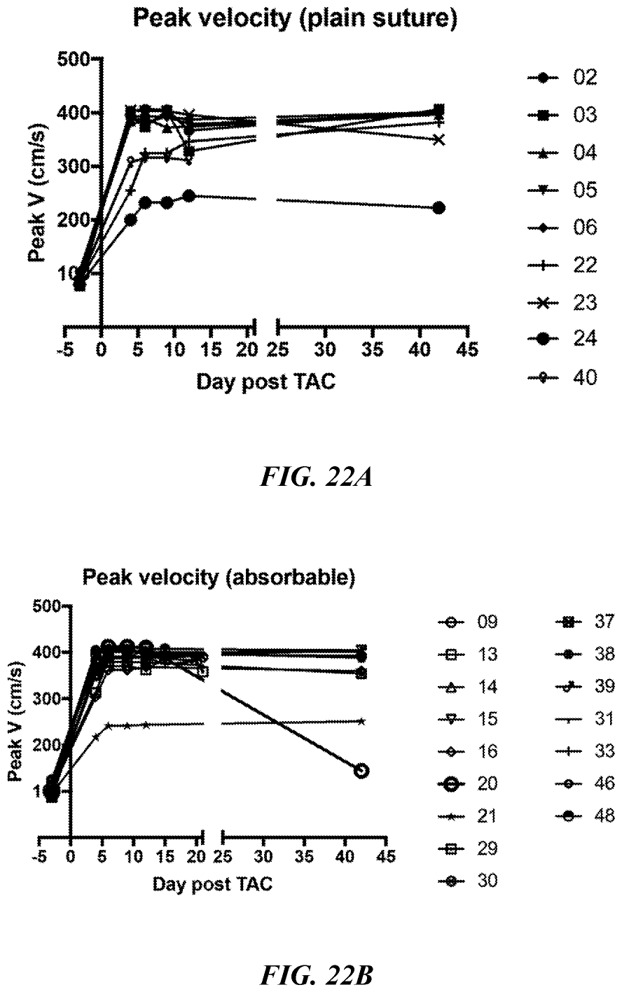

Echocardiogram Data. Echocardiograms assessing peak velocity were performed before (−3 days) and after (4, 6, 9, 12, 15 and 42 days) TAC surgery in order to detect whether or not the suture was absorbed. As shown in FIGS. 22A-B and Table 4 (below), peak velocity (peak V) decreased in one mouse only, suggesting that the suture was totally absorbed at the end of the TAC in that particular mouse (mouse no. 20 in the absorbable group, 20A).

TABLE 4
Echogardiogram data, before (−3 days) and 6 weeks after TAC surgery.

| | Basal (after randomization and | | | |
|---|---|---|---|---|
| | before the surgery) | | End of TAC | |
| | Absorbable | Plain | Absorbable | Plain |
| | (n = 21) | (n = 15) | (n = 6) | (n = 8) |
| Peak velocity (cm/s) | 103.5 ± 2.4 | 89.1 ± 3.0 | 325.6 ± 43.3 † | 370.1 ± 22.0 † |
| Peak gradient (mm Hg) | 4.3 ± 0.2 | 3.3 ± 0.2 | 46.2 ± 9.8 † | 56.1 ± 5.5 † |
| Mean gradient (mm Hg) | 2.4 ± 0.1 | 2.0 ± 0.1 | 22.0 ± 4.3 † | 28.3 ± 2.6 † * |
| LVmass (mg) | 116.1 ± 2.5 | 112.0 ± 3.3 | 170.5 ± 20.0 † | 150.7 ± 6.2 † |
| LV/body weight (mg/g) | 3.52 ± 0.08 | 3.31 ± 0.09 | 5.20 ± 0.90 † | 4.46 ± 0.23 † |
| LVAWd (mm) | 0.70 ± 0.01 | 0.69 ± 0.01 | 0.83 ± 0.04 † | 0.82 ± 0.01 † |
| LVPWd (mm) | 0.69 ± 0.01 | 0.68 ± 0.01 | 0.77 ± 0.04 † | 0.78 ± 0.02 † |
| LVEDd (mm) | 4.42 ± 0.04 | 4.40 ± 0.06 | 4.92 ± 0.19 † | 4.64 ± 0.07 † |
| LV/LVEDd (mg/mm) | 26.19 ± 0.46 | 25.44 ± 0.56 | 34.22 ± 2.85 † | 32.41 ± 1.00 † |
| LVEDs (mm) | 3.174 ± 0.05 | 3.22 ± 0.06 | 3.76 ± 0.32 † | 3.32 ± 0.10 * |
| FS (%) | 28.41 ± 0.76 | 26.809 ± 0.91 | 24.09 ± 3.40 | 28.56 ± 1.34 |
| LVVd (ml) | 0.22 ± 0.01 | 0.21 ± 0.01 | 0.29 ± 0.03 † | 0.25 ± 0.01 * |
| LVVs (ml) | 0.083 ± 0.004 | 0.087 ± 0.005 | 0.148 ± 0.038 † | 0.095 ± 0.008 * |
| EF (%) | 61.4 ± 1.2 | 58.8 ± 1.5 | 53.2 ± 6.3 † | 61.5 ± 2.1 |

LV: left ventricular; LVAWd: left ventricular end-diastolic anterior wall thickness; LVPWd: left ventricular end-diastolic posterior wall thickness; LVEDd: left ventricular end-diastolic diameter; LVEDs: left ventricular end-systolic diameter; FS: fractional shortening; LVVd: left ventricular end-diastolic volume; LVVs: left ventricular end-systolic volume; EF: ejection fraction. Data are mean ± SEM of n mice. Two-way ANOVA analysis (TAC × suture), followed by Sidak's multiple comparisons tests.
(† p < 0.05 versus basal condition = impact of TAC;
* p < 0.05 versus absorbable suture = impact of type of suture.)

With the absorbable suture, the variability of the echo cardiac parameters is higher, creating a tendency to a greater impact of TAC on cardiac properties (except for the mean gradient).

Millar data, 6 weeks after TAC. Millar was performed the day of the sacrifice, in untreated and treated (Fingolimod, 0.3 mg/kg/d, during the last 2 weeks of TAC) mice. Carotid arterial and left ventricular pressure parameters are indicated below in Tables 5-6

TABLE 5
Millar: carotid arterial pressure

| | Untreated | | Fingolimod | |
|---|---|---|---|---|
| | Absorbable | Plain | Absorbable | Plain |
| | (n = 8) | (n = 8) | (n = 8) | (n = 12) |
| Max pressure (mm Hg) | 130.8 ± 12.0 | 130.7 ± 9.3 | 130.6 ± 5.5 | 126.5 ± 4.6 |
| Min pressure (mm Hg) | 68.7 ± 7.5 | 60.0 ± 4.5 | 63.3 ± 2.2 | 62.8 ± 2.4 |
| End-diastolic P (mm Hg) | 68.8 ± 7.5 | 60.1 ± 4.5 | 63.4 ± 2.2 | 62.8 ± 2.4 |
| Max dPdt (mm Hg/s) | 4384.7 ± 425.2 | 4327.90 ± 406.0 | 4603.3 ± 238.0 | 4549.8 ± 325.1 |
| Min dPdt (mm Hg/s) | −3298.4 ± 644.7 | −3973.3 ± 538.3 | −3941.4 ± 439.2 | −3649.6 ± 413.5 |
| Tau Weis (mm Hg/s) | 200.6 ± 15.1 | 187.1 ± 21.6 | 174.7 ± 11.0 | 191.7 ± 15.8 |
| Heart rate (bpm) | 519.8 ± 24.4 | 502.9 ± 21.5 | 536.3 ± 17.1 | 533.4 ± 17.6 |

Data are mean ± SEM of n mice. Two-way ANOVA analysis (suture × treatment), followed by Sidak's multiple comparisons tests.

TABLE 6
Millar: left ventricular pressure

| | Untreated | | Fingolimod | |
|---|---|---|---|---|
| | Absorbable | Plain | Absorbable | Plain |
| | (n = 7) | (n = 8) | (n = 8) | (n = 11) |
| Max pressure (mm Hg) | 139.4 ± 10.5 | 142.1 ± 7.3 | 131.56 ± 9.1 | 131.6 ± 5.2 |
| Min pressure (mm Hg)) | 5.9 ± 4.7 | −0.16 ± 1.7 | 5.8 ± 3.3 | 2.0 ± 2.0 |
| End-diastolic P (mm Hg) | 10.0 ± 4.4 | 3.4 ± 1.8 | 11.1 ± 3.3 | 7.28 ± 2.4 |
| Max dPdt (mm Hg/s) | 8424.5 ± 382.2 | 8314.6 ± 493.9 | 7737.9 ± 829.8 | 8061.8 ± 530.9 |
| Min dPdt (mm Hg/s) | −9300.9 ± 477.5 | −8891.7 ± 469.2 | −7492.0 ± 915.2 | −8293.1 ± 628.3 |
| Tau Weis (mm Hg/s) | 6.4 ± 0.9 | 5.6 ± 0.3 | 7.6 ± 1.3 | 6.27 ± 0.6 |
| Heart rate (bpm) | 524.2 ± 23.3 | 519.8 ± 16.0 | 567.7 ± 12.7 | 546.4 ± 16.9 |

Data are mean ± SEM of n mice. Two-way ANOVA analysis (suture × treatment), followed by Sidak's multiple comparisons tests

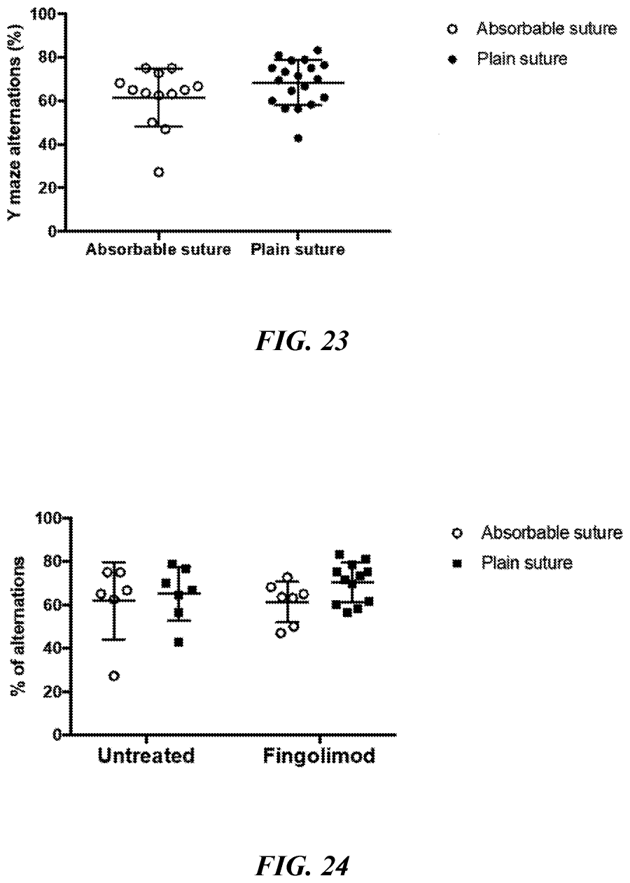

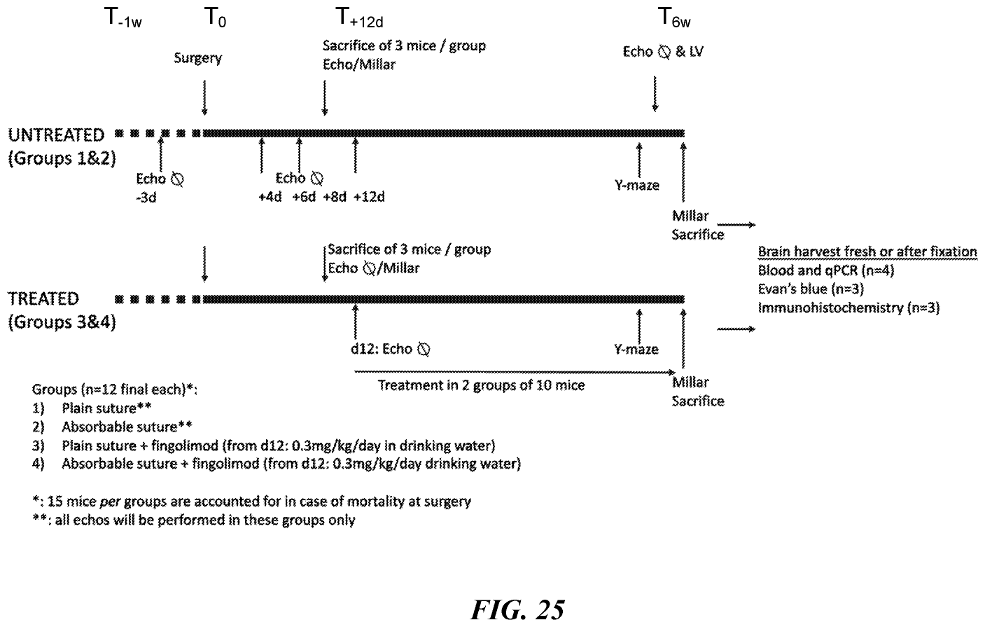

Cognition (Y Maze—exploratory behaviour). Cognition was measured according to the number of behavior alterations within the three (3) arms of a Y Maze, as shown in FIGS. 23-24.

Evan's Blue data. Analyses of the in vivo data above demonstrate that the absorbable suture did not resorb in time, i.e. in 7 to 10 days as advertised by the manufacturer Ethicon, with the exception of mouse 20A. Consequently, there were no statistical differences between the two groups (plain vs. absorbable suture). Likewise, for the other two fingolimod-treatment groups. Nonetheless the mice were treated for the last 2 weeks (starting 4 weeks post-TAC surgery) despite the lack of evidence of normalization of the aorta diameter based on repeated echo assessment, as discussed below.

Twenty three (23) mice were treated with fingolimod (0.01 mg/day in drinking water, i.e. 0.3 mg/kg/d) for the two last weeks, i.e., 4-week TAC post-surgery. In the initial protocol, it was planned to treat the mice after the absorption of the suture estimated at 12 days after the surgery, and thus for 4 and a half weeks. Because absorption of the suture this did not occur, it was decided to treat the mice for the last two weeks even if the damage was expected to be well established.

The images were reconstructed to acquire the whole right hemisphere in order to quantify Evan's blue extravasation. The analysis was performed by a blinded technician using Image J® and background fluorescence filters. Four mice were untreated (23A, 24A, 30A, 40A) and three mice were treated (41A, 42A, 52A). FIG. 26A represents all data acquisition; then the normality and Log normality of Evan's bleu data was tested as shown in FIG. 26B.

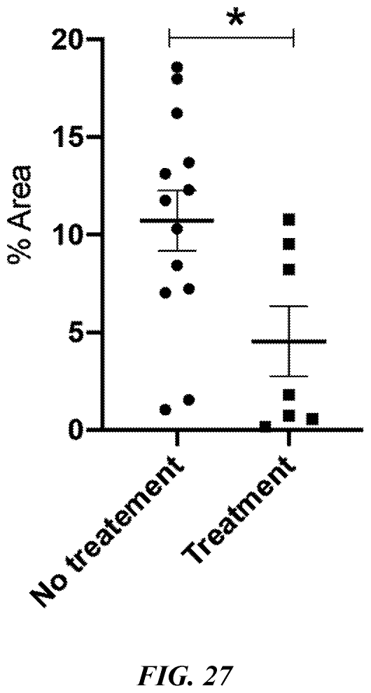

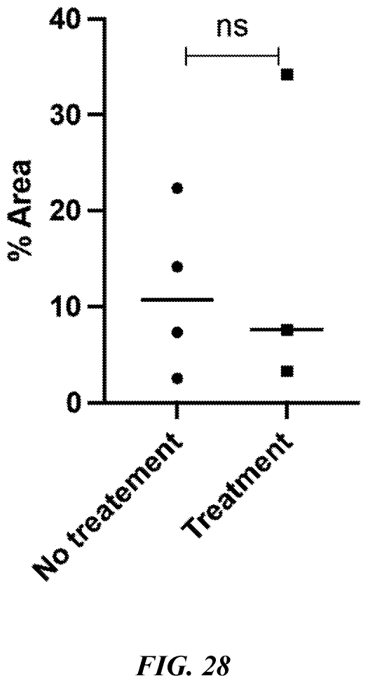

With computed-generated exclusion of abnormal distribution of individual data of each region-of-interest (ROI) from individual mice, the dot plot shown in FIG. 27 suggests that fingolimod somewhat protects the cerebrovascular endothelial integrity by maintaining the barrier function of the endothelium. However, when all values from individual mice are averaged without exclusion of abnormal distributed values, the trend remains, but statistical significance is lost due to the low number of animals included in each group (including outliers), and the large variability of Evan's blue diffusion.

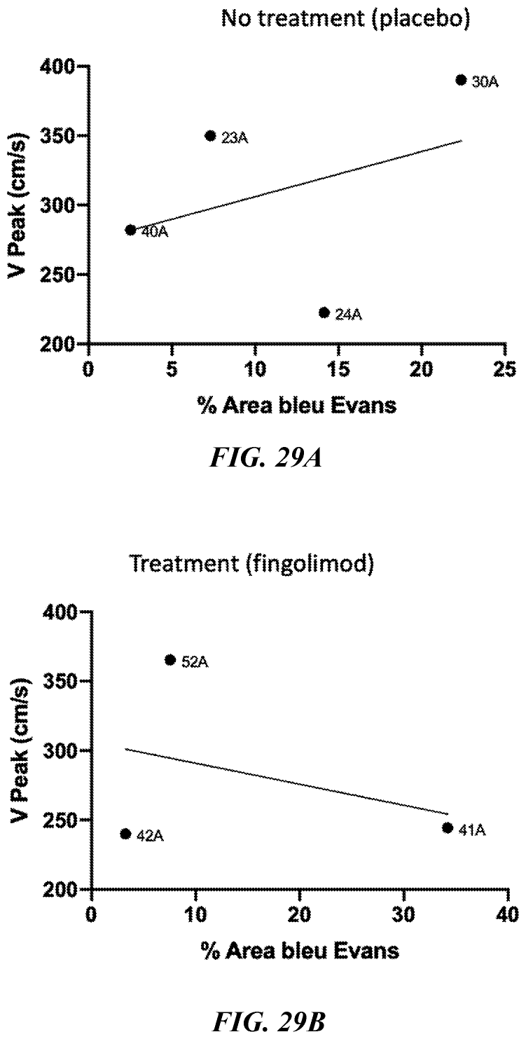

All mice from the two groups (treated and not treated) had the same velocity peak (a proxy of pulse pressure) measured by echocardiography (see FIGS. 29A-29B), thus the potential beneficial effect of the treatment cannot be attributed to a lower pulse pressure in the brain.

In summary, the S1P₁ receptor ligand is likely to be protective by preventing the increase in BBB permeability associated with the rise in cerebrovascular pulse pressure. Additional testing may be performed in a larger cohort of mice, with normalization of cerebrovascular pulse pressure following the absorption of the suture. The treatment would be initiated after only a week of high pulse pressure at the same time of the normalization of the pulse pressure to maximize efficacy.

qPCR analysis. Targeted mRNA expression was performed in the microvascular fraction of the brain. Two approaches were followed: either the quantification (RT-qPCR) was performed in the microvascular fraction of the whole brain (not treated or treated, n=5 per group, unpaired t-test) or after isolation of the ipsi- and contra-lateral hemisphere of the brain of 3 mice treated or not (paired statistical analysis (left vs. right) and unpaired t-test (no TT vs. TT)). The targeted gene transcripts are discussed below.

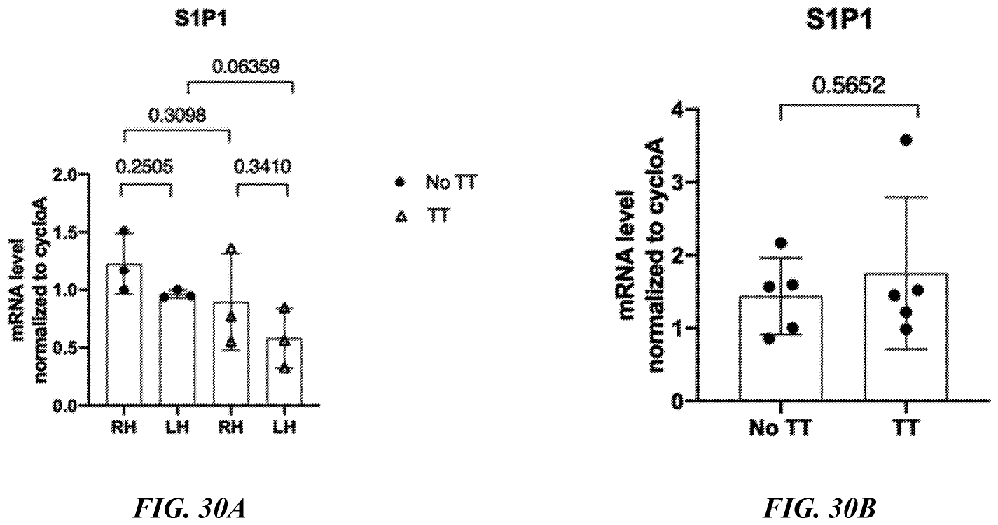

S1P1 (see FIGS. 30A-30B): G protein-coupled receptors and is highly expressed in endothelial cells. It binds the ligand sphingosine-1-phosphate with high affinity and high specificity, and suggested to be involved in the processes that regulate the differentiation of endothelial cells. Activation of this receptor induces cell-cell adhesion.

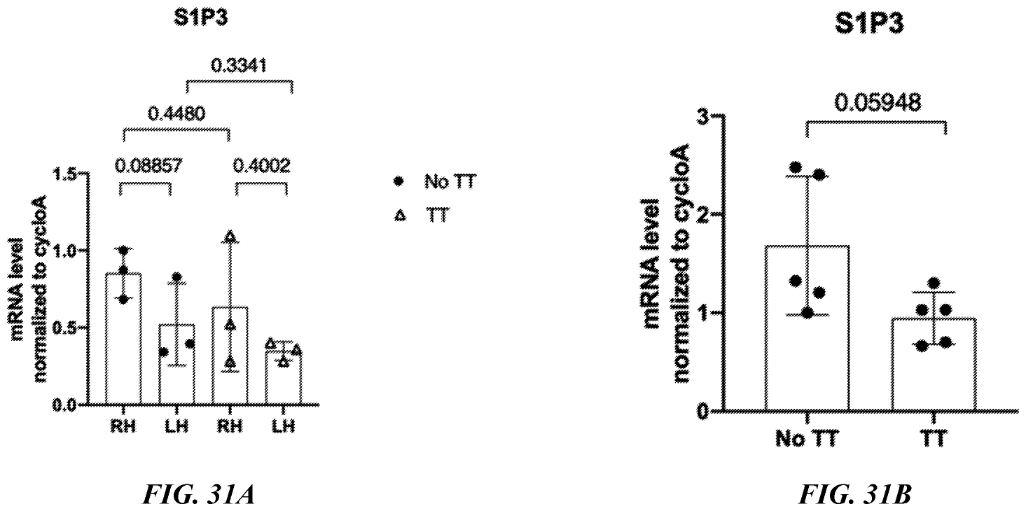

S1P3 (see FIGS. 31A-31B): This gene encodes a member of the EDG family of receptors, which are G protein-coupled receptors. This protein has been identified as a functional receptor for sphingosine 1-phosphate and likely contributes to the regulation of angiogenesis and vascular endothelial cell function.

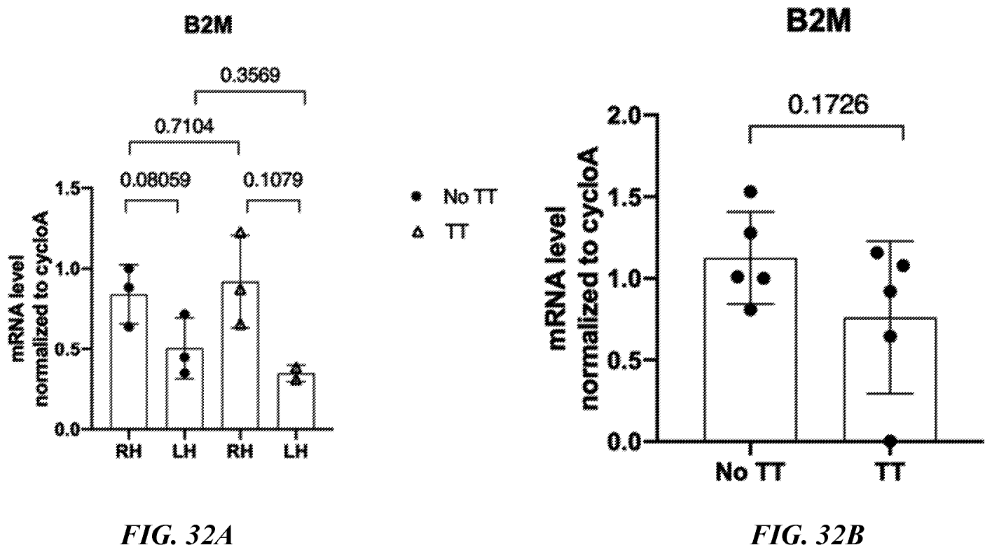

B2M (see FIGS. 32A-32B): β₂ microglobulin also known as B2M is a component of MHC class I molecules, MHC class I molecules have α₁, α₂, and α₃ proteins which are present on all nucleated cells.

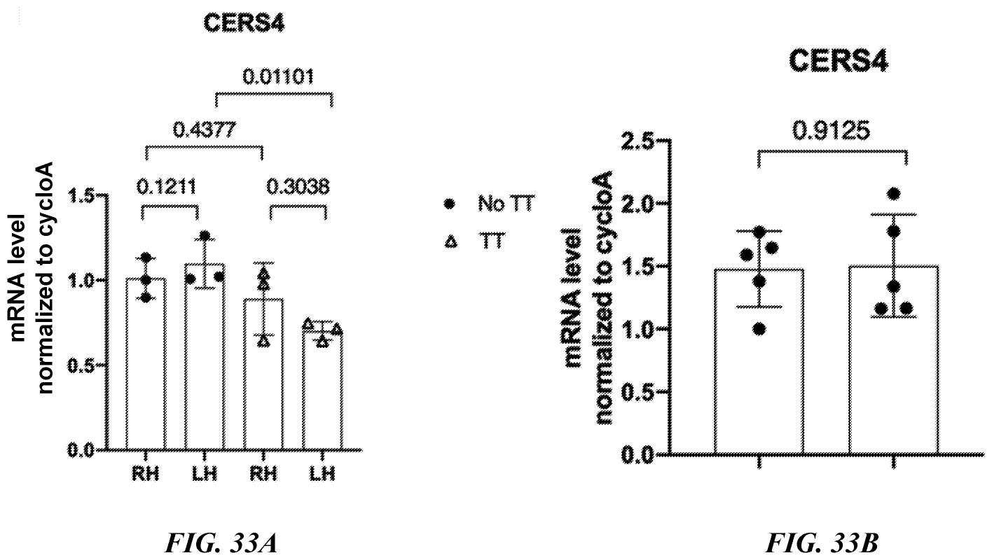

CERS4 (see FIGS. 33A-33B): Ceramide synthase that catalyzes formation of ceramide from sphinganine and acyl-CoA substrates, with high selectivity toward long and very-long chains (C18:0-C22:0) as acyl donor.

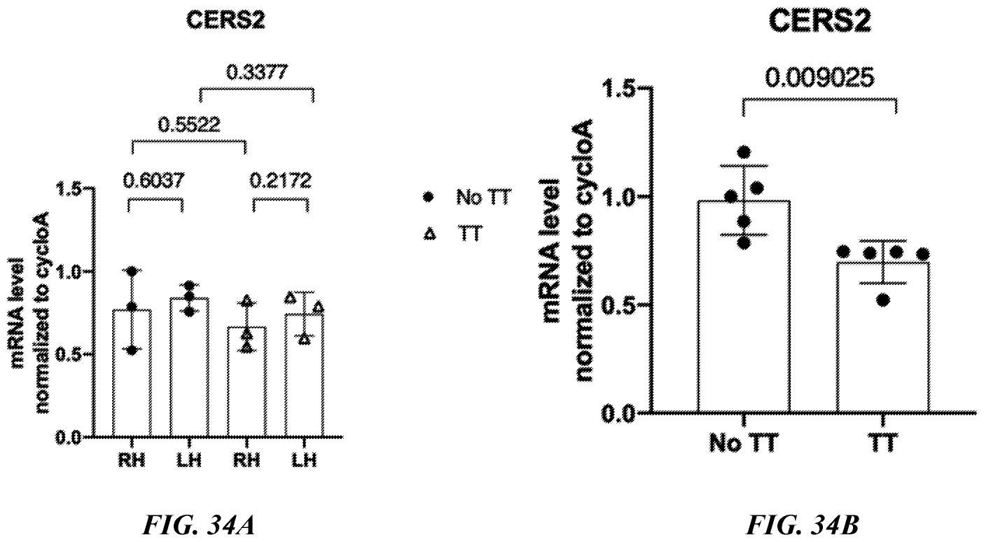

CERS2 (see FIGS. 34A-34B): Plays a non-redundant role in the synthesis of ceramides with very-long-chain fatty acids in kidney, liver and brain.

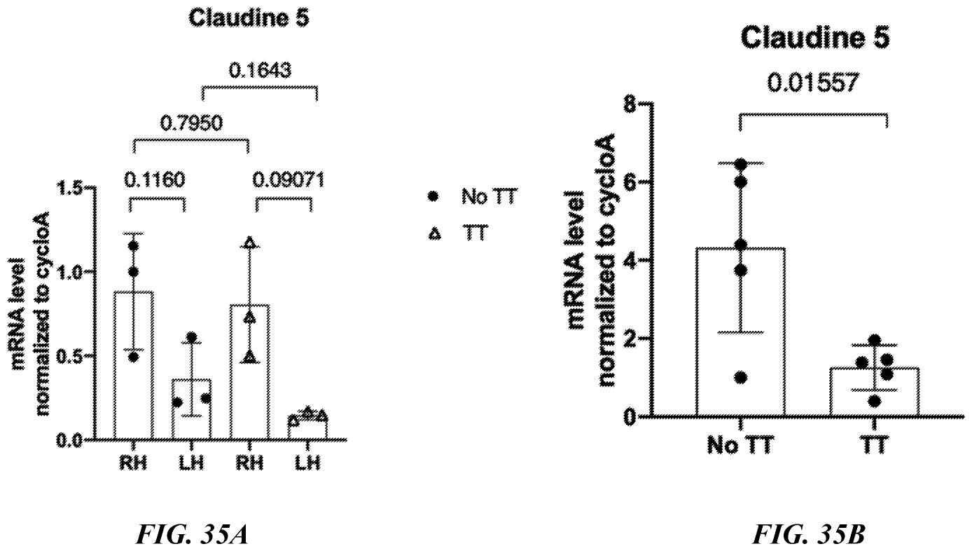

Claudin (see FIGS. 35A-35B): Claudins function as major constituents of the tight junction complexes that regulate the permeability of epithelia.

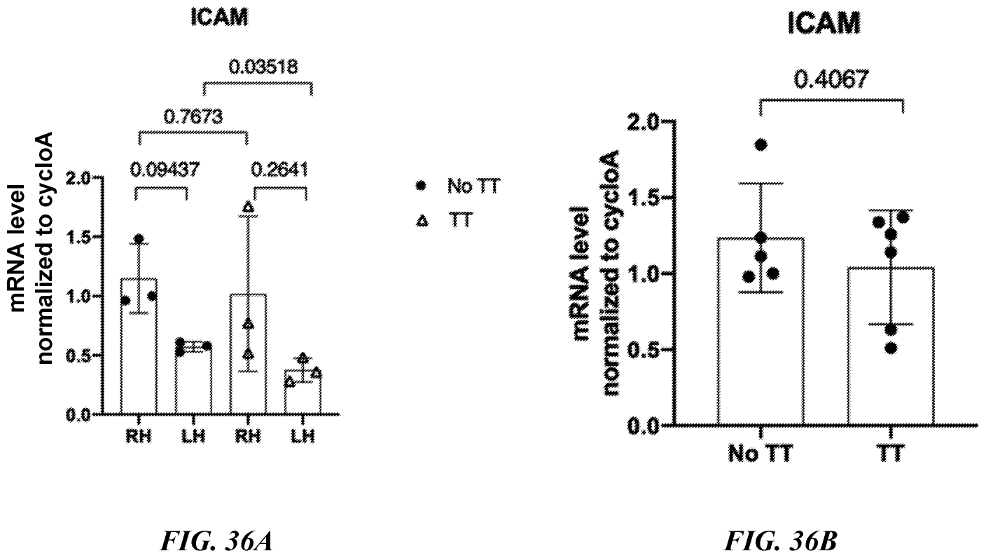

ICAM-1 (see FIGS. 36A-36B): Intercellular Adhesion Molecule 1. ICAM proteins are ligands for the leukocyte adhesion protein LFA-1 (integrin alpha-L/beta-2). During leukocyte trans-endothelial migration, ICAM1 engagement promotes the assembly of endothelial apical cups.

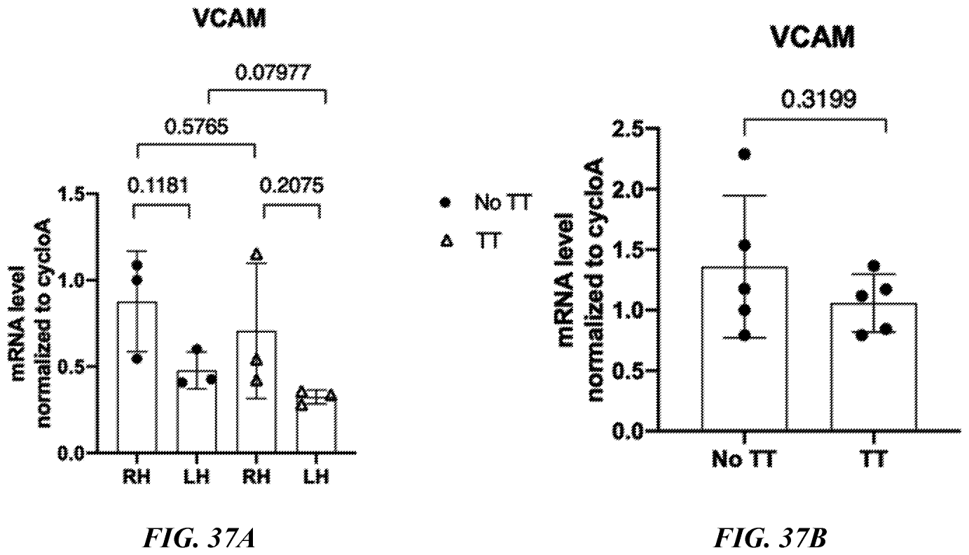

VCAM-1 (see FIGS. 37A-37B): Vascular Cell Adhesion Molecule 1. Important in cell-cell recognition. Appears to function in leukocyte-endothelial cell adhesion. Interacts with integrin alpha-4/beta-1 (ITGA4/ITGB1) on leukocytes, and mediates both adhesion and signal transduction.

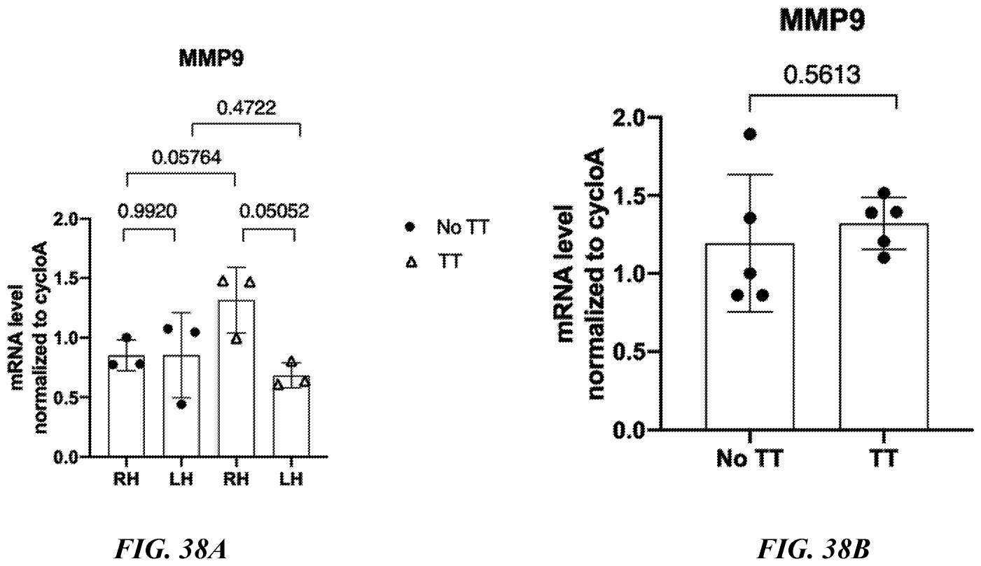

MMP9 (see FIGS. 38A-38B): Matrix metalloproteinase that plays an essential role in local proteolysis of the extracellular matrix and in leukocyte migration. Cleaves type IV and type V collagen into large C-terminal three quarter fragments and shorter N-terminal one quarter fragments. Degrades fibronectin but not laminin or Pz-peptide.

Cyclophilin-A (CYPA) has been used as the housekeeping gene. It also known as peptidylprolyl isomerase A (PPIA), which is highly abundant and found in the cytosol.

The S1P₁ receptor ligand has complex effects on the brain microcirculation. It has a tendency to decrease S1PR1 and S1PR3 mRNA expression in the ipsilateral hemisphere and globally, respectively; this could represent a downregulation of the receptors in response to the chronic exposure to the agonist. CERS4 expression is decreased in the treated ipsilateral hemisphere, while globally, CERS2 is decreased; again, it seems related to the treatment and the downregulation of the synthesis pathway. More difficult to explain are the following: Claudin-5 expression is globally significantly reduced by the treatment, suggestive of a reduced tight junction between cells. The effect of the treatment on MMP-9 is ambiguous, with a tendency to be increased in the contralateral hemisphere, while ICAM-1 is reduced by the treatment in the ipsilateral hemisphere accompanied by a similar tendency in VCAM-1 expression. Immunofluorescent staining of the brains should provide evidence whether the treatment has protected the ipsilateral hemisphere from the rarefaction in capillaries incurred by the acute and prolonged increase in pulse pressure.

### Discussion

Under a condition of high pulse pressure, Fingolimod appeared to protect the Blood Brain Barrier from excess permeability. Certain cellular analyses (e.g., of Cell Adhesion Molecules) supported the possible benefit of this drug, in this setting. Further experiments may be performed, with optimized “transient TAC” conditions and higher numbers of mice. Taken together, the data are encouraging of a combined approach to BBB protection.

### VIII. CONCLUSION

Although many of the embodiments are described above with respect to systems, devices, and methods for treating and/or slowing the progression of vascular and/or age-related neurological conditions (e.g., dementia) via combinatorial therapeutic agents (e.g., drugs) and intravascular methods, the technology is applicable to other applications and/or other approaches, such as surgical implantation of one or more damping devices and/or treatment of blood vessels other than arterial blood vessels supplying blood to the brain, such as the abdominal aorta, in combination with one or more drugs. Any appropriate site within a blood vessel may be treated including, for example, the ascending aorta, the aortic arch, the brachiocephalic artery, the right subclavian artery, the left subclavian artery, the left common carotid artery, the right common carotid artery, the internal and external carotid arteries, and/or branches of any of the foregoing. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 2A-20.

The above detailed descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method for treating and/or preventing one or more effects of a condition in a subject in need thereof, the method comprising:
providing a device for treating and/or preventing one or more effects of the condition,
and configured to be placed in apposition with a blood vessel, the device comprising—
a flexible damping member forming a generally tubular structure having an inner surface and an outer surface, the inner surface formed of a sidewall having one or more at least partially deformable portions, and
an abating substance disposed within the one or more at least partially deformable portions of the sidewall configured to move longitudinally and/or radially within one partially deformable portion in response to pulsatile blood flow within the blood vessel; and
providing a therapy that treats or slows one or more effects of the condition in combination with the device, wherein the therapy is an EDG Receptor Family Modulator, an MMP inhibitor, or a senolytic agent or combination thereof.

2. The method of claim 1, wherein the EDG Receptor Family Modulator is an S1P receptor agonist selected from fingolimod, siponimod, ozanimod, or ponesimod.

3. The method of claim 1, wherein the MMP inhibitor is doxycycline or dexamethasone.

4. The method of claim 1, wherein the senolytic agent is dasatinib, quercetin, or a combination of dasatinib and quercetin.

5. The method of claim 1, wherein the therapy prevents and/or reduces abnormal cleavage of amyloid precursor protein in the subject's brain, prevents and/or reduces expression and/or accumulation of β-amyloid protein in the subject's brain, decreases inflammation, reduces and/or prevents oxidative stress, reduces and/or prevents ischemia, prevents dysregulation/damage and/or death of a neuron, prevent dysregulation and/or damage to the blood brain barrier, eliminates senescent cells, improves tight junctions, inhibits MMPs, and/or induces transcriptional activation of TIMPs.

6. (canceled)

7. The method of claim 1, wherein the therapy is provided at a first dosing regimen which is less than a second dosing regimen that is provided in the absence of the device.

8. The method of claim 1, wherein the therapy is provided via a first route which is different than a second route that is provided in the absence of the device.

9. (canceled)

10. (canceled)

11. (canceled)

12. The method of claim 1, wherein the device has a low-profile state and a deployed state, and when in the deployed state, the sidewall is generally tubular.

13. The method of claim 1, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

14. The method of claim 13, wherein when positioned in apposition with the blood vessel and a pulse wave travels through the blood vessel, the flexible damping member applies a stress at the first location along a length of the tubular structure.

15. (canceled)

16. (canceled)

17. (canceled)

18. The method of claim 1, wherein the inner surface and/or an outer surface has a generally cylindrical shape or an undulating shape that undulates in a longitudinal direction.

19.-84. (canceled)

85. A system for treating and/or preventing one or more effects of a condition in a subject in need thereof, the system comprising:
an effective amount of a therapy for treating and/or preventing one or more effects of the condition, wherein the therapy is an EDG Receptor Family Modulator, an MMP inhibitor, or a senolytic agent or combination thereof
a device for treating and/or preventing one or more effects of the condition,
the device comprising—
a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
an abating substance disposed within the one or more at least partially deformable portions of the sidewall configured to move longitudinally and/or radially within one partially deformable portion in response to pulsatile blood flow within the blood vessel;
wherein, the effective amount of the therapy for treating and/or preventing one or more effects of the condition is carried by or more of the at least partially deformable portions of the device, and
wherein, when the one or more at least partially deformable portions are at least partially deformed, the effective amount of therapy for treating and/or preventing one or more effects of the condition is released from the device.

86. The system of claim 85, wherein the effective amount of the therapy further comprises a first effective amount of the therapy and a second effective amount of the therapy.

87. The system of claim 85, wherein the second effective amount of the therapy is greater than the first effective amount of the therapy.

88. The system of claim 85, wherein, in response to a first pulsatile blood flow within the blood vessel, the one or more at least partially deformable portions are at least partially deformed to a first degree of deformation.

89. The system of claim 88, wherein, in response to a second pulsatile blood flow within the blood vessel, the one or more at least partially deformable portions are at least partially deformed to a second degree of deformation.

90. The system of claim 89, wherein the second degree of deformation is greater than the first degree of deformation.

91. The system of claim 90, wherein the first effective amount of the therapy is released from the one or more at least partially deformable portions in response to the first degree of deformation.

92. The system of claim 90, wherein the second effective amount of the therapy is released from the one or more at least partially deformable portions in response to the second degree of deformation.

93.-112. (canceled)

113. A method for treating a patient having a condition comprising:
(a) determining or having determined whether the patient has an elevated pulse pressure or elevated pulse wave intensity/FCWI and a history of a microbleed, blood brain barrier dysfunction or permeability, systemic inflammation, blood brain barrier inflammation, neuroinflammation, increased level of at least one circulating or CSF cytokine, increased reactive oxygen species, or a combination thereof by:
(i) obtaining or having obtained information which indicates that the patient has the elevated pulse pressure or elevated pulse wave intensity/FCWI and has previously had symptoms of a microbleed, blood brain barrier dysfunction or permeability, systemic inflammation, blood brain barrier inflammation, neuroinflammation, increased level of at least one circulating cytokine, increased reactive oxygen species, or a combination thereof and/or was previously diagnosed with a microbleed, blood brain barrier dysfunction or permeability, systemic inflammation, blood brain barrier inflammation, neuroinflammation, increased level of at least one circulating cytokine, increased reactive oxygen species or a combination thereof; and/or
(ii) monitoring or having monitored the subject for the elevated pulse pressure and symptoms of a microbleed, blood brain barrier dysfunction or permeability, systemic inflammation, blood brain barrier inflammation, neuroinflammation, increased level of at least one circulating cytokine, increased reactive oxygen species or a combination thereof; and
(b) if the patient has previously had symptoms of a microbleed, blood brain barrier dysfunction or permeability, systemic inflammation, blood brain barrier inflammation, neuroinflammation, increased level of at least one circulating or CSF cytokine, increased reactive oxygen species, or a combination thereof, was previously diagnosed with a microbleed, blood brain barrier dysfunction or permeability, systemic inflammation, blood brain barrier inflammation, neuroinflammation, increased level of at least one circulating cytokine, increased reactive oxygen species, or a combination thereof, and/or symptoms of a microbleed, blood brain barrier dysfunction or permeability, systemic inflammation, blood brain barrier inflammation, neuroinflammation, increased level of at least one circulating cytokine, increased reactive oxygen species, or a combination thereof were monitored, then
(i) providing an EDG Receptor Family Modulator, an MMP inhibitor, or a senolytic agent or combination thereof. to the patient, and
(ii) providing a device for treating and/or preventing one or more effects of the condition,
the device comprising—
a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
an abating substance disposed within the one or more at least partially deformable portions of the sidewall configured to move longitudinally and/or radially within one partially deformable portion in response to pulsatile blood flow within the blood vessel;
(c) if the patient has not had symptoms of a microbleed, APOE4 expression, blood brain barrier dysfunction or permeability, systemic inflammation, blood brain barrier inflammation, neuroinflammation, increased level of at least one circulating or CSF cytokine, increased reactive oxygen species, or a combination thereof, was not previously diagnosed with a microbleed, APOE4 expression, blood brain barrier dysfunction or permeability, systemic inflammation, blood brain barrier inflammation, neuroinflammation, increased level of at least one circulating or CSF cytokine, increased reactive oxygen species, or a combination thereof, then providing the device for treating and/or preventing one or more effects of the condition.

114.-131. (canceled)
